SEQUENCE LISTING (11 sequences)

SEQ ID NO: 1 - DNA, 14836 bp, Mus musculus
cctcccccgtgtctccccacacccgggttggggttgttttggttgaccagagtggaacacaacgatctattggcagggctgaacaccaatgggtctatttgtaaagcgccaatgaccactttctgaagcagggttttagggagcggggccttagggaactctttggtcctttttagaacactggactttcttctggaaaggcaggaaacactgaagtttaagaagttgtttccagcttccattaactgaacacacattaaaaccaagcacagagaatcaggacgtttcgcgggagtgagacccagtcatttctcctccgtttccattctgcagggtgagagttgtaatcacccacccactattcgtaccatccacccacccccagtcgagagaataggggtacagaggggaggtggcaaagaaaattcacgatactgagtatctctgggagacctgtttggtctctttgctcggtagcgcagccctacgttagaatgcatcttcccgggaatgactgtagtgagactttggctgggaatccaagttattctaactgtagattggtccacgttgccctaagcctagcagtccactgcggcacagacaccctggacatgaggtgggtcagcttaagttcctggcacgaaagaaagggtactctggcaacttttggatgcggcgaaacagactgtttcgtctctcaggttcttatttcacggcttgtgcctttgacagccccttagtttctctatctgcaggatgggagcattaagctctacgacccagcctctttacaattcaggtccaaagagcccgcccaagttggggactgggaagatcaaaggtctcagcacccagcggagccgcggacactgagggcgccaagaagggggtgggtaggtagggaactggaagggcggctgctccgcaggggatgcgcgtcagagaccccagccacactccaggcccgccccttgatgagccccgccccgccccgcctggttttcgcctctaaagcgcccagcgctcgcctcccgctgccgcactttcactctcggtccacctcggtgtcctcttgctgtccagctctgcagcctccggcgcgccctcccgcccacgccatggacgccaaggtcgtcgccgtgctggccctggtgctggccgcgctctgcatcagtgacggtgagtgcaatccgcggccgggcccgggaaaggctcgcagctctgcgccggagctccttcgggtccgcggttcctctgcccgcgccgaagtcgcggagaaagaactcggtcggcgccgttcactacaagcgaacttggggcagtccactttgcagggcgcactcccaccgggtgccctttcccgtgtcccacgggtcgcaccgaggttttgtgctctgcgaagtgcggccataggacctagagagggctgcaggggaggacccgcaggattgttgggcaagagtgggttcggcgcggaatggaagcgtgggcgattgtgtccggggcttgggccccggagcgcgccagctgcactcagctagtgtctaccggcgcccagatgtttccagaggcgaagggcagcgcggtcccggagttgaccgtgcaagaggttcactcgggtggtgcgtgtgtcagcaaactctcaaagaccggtcaagtagctcgaagtgcatggcttggctataggttcagtggtgaggctgagtttcgtcccctgcgggtgtagcgtgttctcttacagcaccctcgaggggctcagggccaccagcagcgcagcgcagctcttgaactcgcgctgccagccagggccgcgcttctgcacagttcgttggtccgtagcgacgcggacctgagcacgcgtctcttcactgcccctttttcttcttacccgggtcactagacaaaggctcagcagttacccaagctatatgcacacctctccccaacccccaaacacacctgcaaacgggcgcttttgtagccagccccggagtcctcagctctggaatgagagctgcagcggagttcagtctcccagacccagggtggtgtcttctttcactgggaaagggctttcattttgttttctttttttgacactgaagagaaaactctcagcgctgttacaagacaccgttgctgcaaaacaaaacaaaccattgcctctgaacacaaaacaaaatcctactagtcgatcccctgccttcctccgcagtggtgtttcctggagagaactgagggacagtcggggctcttggtgagactgagctctaaatgctgcccaagtacaccaactcgttcgtttgggttctttccctgtgacaacggggtacgggaatggttggagttgcctagtccgagggaaatgttctgtaaaagaatagtcagttgctgatcggagtagtaaaaaaaaagaaatgaaaggcagtttcgatttttttttttttttttttttttttttttgttaccgagaacacccgggaggctgagccttcccactggtcccccagtgccccgtcatggagcacattgatttgggcattaataattgaatgagctggtgatgttgcaagggtcacagcctctggcaagttaggtatggggcaagaatgtaggactcaggtcctcaaggttggagtgcaattatccagagtaaaagttgtctcaccctcaacatattctgaccctaggaagagtcggattgttgacagtgtctggatcagacctgttctctaggcaggaccccattgtgctgcccgaatgaacttttttacctcctagtgcctgtgtgccctctgatcttacacagccctcaagttgcagcacggctaaccttgctgtggttcctgtcttttcccatcagctactccaactcagaagctagatagtagacaccggaggcttctttggttaaacccagagcagcaggcttgccaggcttgttagattgaatggacccctggttccctaagccaagctctctagattcccaagtccagggtggcagcagagctggattagactttggtctgtacctgaagtctggttttcctatgctttagagtctaaagacactacccttcctggggcatgcatcccttagctaaataatgcttgcagaagaagataatcccatcatatatttaattcggtccacttctccagctgcttcccaaaggcagtgaacttcagaatacccagaagtctcctggaactctaaataagcaaacttaaaatcctggggctaactattctcagtcatacttttaaactttggtgaaaagacccataaattgaaacatttggggatgctcagtagagctaggataaaaccctgttgttgggggagcagctacaaatccagcagtcctcagggtttgcaattctagacttaaagggtggttcttaagggggggttctaaaggagccccttgctaatttacactaatgagtgtcaattatagcattttgcaaattggtgaattggcaaacaaagctggtaataggatccaggaggcctaggcatccaggtagtgaccataaaagccacggttgaccccagcttttgggaaaagctggatagaaggtaaatccgggtcctcccctctggattcttttgtgatttccagggcttaggatagggtgagtgggaggagggaaaactgcaggtggtagaagtgaagccccccacctccaggcctgcaccagagggccacaagggagcccagaactctgccaccccacttctcctgggtccttttgtcctttagaggctgagcccagtcagatctcactgtgatccctggccgaggggatggtctttgcaagaaactttctgtaaccattcctgctgatgttcctgagtcttccccacaagagccaccaaaccccctgcaccaggcagataatgactggccccacttttctctctacacctcctctaggtaaaccagtcagcctgagctaccgatgcccctgccggttcttcgagagccacatcgccagagccaacgtcaagcatctgaaaatcctcaacactccaaactgtgcccttcagattgtgtaagtcctagccgccatcccccaaagaggagcatggtatagaagcctcggacttggcataactaggggcagctgttaccaccaccaccacggggacactgatatgccatcagacatgggtttcaaaggatacttttgttccccagagccctgatgtcctcagtgtttctcactcttgctttccaagctgtttcttgcagcacagtgggccgcctctctacagaaaaagccatggacttgatggaggtcagccctcagctgacagttgggtctgtcttgtcagtttcaaggttctggtgtccaaagttaatcctttctcacatagaaaaaaaaattacaagacccggatggcacggggggggggggggttcagttttactcacttgcactcacttgctcagaggtcatttttgttttagagttttagagtttgctggagtgtgatggtagctgccagtatttgatttaaatttacctgggaaataagaaaagcccaaaaaaggtataaatgatgtgaatatctcactcagagtctggtagacttggcagagatgtgtcctgtgctagtctgtcctgctcactgccccccagcaggggttcccatcctcgggagactcaacactaacaacagtataaggatgcagcagctggagcaatgctagcctgacggctttgtcacccaacggtgactgcttcagactttctgtgctcatcagccttcctctccagcctccgctgctgtgttatgtacagtaggctttagagacctagatgatgaatattatttttgctgttttgattaaaatacaatactctcccgagaaagggattttaaagatgatgagtttacgtttgaataggctgtgctggtgcactgtcccgggaagggcccttgaacttagagggtcaaatacaactattgattctgggtgatcactaagttaataaatggcaggatccagactgacacccctgatccctgttgaagttacatccctctgaacgactggtcaactgcagggcagcctgcttgaagagggttacctgtccctaggacactgaacaggcatttgtttttcctagaagacagttcaccagctggagaggagtcgtctcccgtagtttctgtttggttgcttttggtttttgtttggttttggttttttaattatctggcatccaggacttgatggaaaataaccagagctaagctcaccggttcatctgcccattaggaagttctagggatgggagaaagaacacggcgtcaattaacaaatccacaaagctaagaccttgaagcattctgtgaacttgtaaacgcgctcaggcaaccattggacaatttgtctagactgctccttgcccacctgaactgccctgttcctccccttctggactcctgccgtcttcctccagagctacctttaaggttgtcccatgtactatcaaggtgctctgtcaaaagttcttaggctgcttctggcactctccagaattttccaagacctcccccccaccatgatatcagtcatccgcgccttctgggtggttcttcctccacaccctttgggcactttgactcctgtgggatattcgtccttccttttcctttagctttcctcacttgccaagctccaacttggccagaagctcaaatgcctccactgtggtctcttctctgtgtcccctgggagacatccttagcacgtccctaactctgcggtggtggtcccaacacgattcaagtgctatgtcttccaaaactgaagcttccgggagcagcagctgggccctgcagtgaggacctttagctgggtgtgttgggtgagcccacaggatcgctttctcccgcttggctgtacagcgtctctccccttgtgttttggcagtgcacggctgaagaacaacaacagacaagtgtgcattgacccgaaattaaagtggatccaagagtacctggagaaagctttaaacaagtaagcacaacagcccaaaggactttccagtagacccccgaggaaggctgacatccgtgggagatgcaagggcagtggtggggaggagggcctgaaccctggccaggatggccggcgggacagcactgactggggtcatgctaaggtttgccagcataaagacactccgccatagcatatggtacgatattgcagcttatattcatccctgccctcgcccgtgcacaatggagcttttataactggggtttttctaaggaattgtattaccctaaccagttagcttcatccccattctcctcatcctcatcttcattttaaaaagcagtgattacttcaagggctgtattcagtttgctttggagcttctctttgccctggggcctctgggcacagttatagacggtggctttgcagggagccctagagagaaaccttccaccagagcagagtccgaggaacgctgcagggcttgtcctgcagggggcgctcctcgacagatgccttgtcctgagtcaacacaagatccggcagagggaggctcctttatccagttcagtgccagggtcgggaagcttcctttagaagtgatccctgaagctgtgctcagagaccctttcctagccgttcctgctctctgcttgcctccaaacgcatgcttcatctgacttccgcttctcacctctgtagcctgacggaccaatgctgcaatggaagggaggagagtgatgtggggtgccccctccctctcttccctttgctttcctctcacttgggccctttgtgagatttttctttggcctcctgtagaatggagccagaccatcctggataatgtgagaacatgcctagatttacccacaaaacacaagtctgagaattaatcataaacggaagtttaaatgaggatttggactttggtaattgtccctgagtcctatatatttcaacagtggctctatgggctctgatcgaatatcagtgatgaaaataataataataataataataacgaataagccagaatcttgccatgaagccacagtggggattctgggttccaatcagaaatggagacaagataaaacttgcatacattcttatgatcacagacggccctggtggtttttggtaactatttacaaggcatttttttacatatatttttgtgcactttttatgtttctttggaagacaaatgtatttcagaatatatttgtagtcaattcatatatttgaagtggagccatagtaatgccagtagatatctctatgatcttgagctactggcaacttgtaaagaaatatatatgacatataaatgtattgtagctttccggtgtcagccacggtgtatttttccacttggaatgaaattgtatcaactgtgacattatatgcactagcaataaaatgctaattgtttcatgctgtaaacctcctaccgtatgtgggaatttatttacctgaaataaaatctactagttgttagatggagtgcacatacatttctgaagatggagaaaaacaggtgtgcctgctgatcaggtgctgtgggctgccctgcagtcctggtgagcgacagacactgaggcaggcttgtctcatgaacaggctgcctctgcagtgaaagtttttgtgtattttttttaacccaagctagttttctaatgaataatacttgactcactaatttcccctcctcctccttctcctcagttctcctaacatcctcatgtgatccccagactcaactccagtaatatcaagctttcctattttcccatgtaaaaaaatcccatgactctgggccatgttaatatcaggcttttgtgggaacaggtggcctcaccccataaatcattaaataccattcagcttgaatcattttaatgtgacagtcacaaaccagttgctctaataaaaactctgctaaccatccttctccttagctctctagaacaatctcagttatccctagggatgctccccagcatccagaaaagagaagtgggatcaatcatcctgcctttctccccctcctctcttggagggctgcctgagcccgtggcctccacctcccctgctttgtataatttgaaatgcagatttgtagtgaaggcagagttcacctctgcattgaaagggaaggcaggcccagagcttccttccctgccctctgagatgtgcatttatgtctcaggatggatgagctttggtaggaatgctcaaaaccaggaccagccagacaaactggcagtccctgtaagcggttcccgggtcatagggttagggcacccctgtttaactttggggtggggaaagtatctggttttctttgataaattgcttgtgaaccacatttgccaagtggcctccaggcctcaaactcaaagaccgagctaaatcgactcggaaggcaatgctgaatgaagattgtgggaactgagatagatacactcctctatgttgcaatgtgattaatggttctactaattttatctaagggggcgcagagaagaaaaagtggggaaaaaagaaaagataggaaaaaagaagcgacagaagaagagaaaggctgcccagaaaaggaaaaactagttccccgcttcctgccgatggaccgcagtgcgctctgctctggcgctttgtaactcgctcctccctcttcgggggcagaccccacactccgggcaggtgctcaaacctgacggtaaactcttccctcttcgggggcagaccccataccccggggcgggtgcttaggctttcctgccctggtggccacaccagctgctgtatttatgtgcttcataaggccctgctctgtctgctaaagctatgaagaaagatgtgcagagactggggtggagactaagccaaagaggagctgcctagcctggcagcattgccccgagctgagcccccttggccaggacttcacaaggctcacacctacaatcccatgaaggccagggtggtctgcttagccaggaaagggcaagtgccttcccctcggccacactgccccttgtggccttctcgggacatgtggtaactgacttgctctcaggcccacccgcagcttttccaaatacctgcagccttcagccctgctgccctgcctgtgggagcagctttgactccagtccagaagggtttctgcagactgtgttgggtgagacgcagaaaggatgaaatctcagaacacatgtcagctgcttctcaggaaatcttttctttggacaattcactttagagtctttaaacgggtctctcgtggggaggatagatgtgctctggaactttctgaaggaccagcagcttcagggactcttagtctgtccttccccacttttggtcccaacatccctgggatggtgtgctgtctgggcaccacggtctccatcctcactcctgagagatttctgccttctgtgagttgggttaaagctctggaattatctactatcccaatccactaccctcacctggcaatatttgtctgtttttgtttgtttgtttgtttgtttttgtcttttgccagtttgaattagaaggcaaggctctgattttagtagtgttttggaaaaggacttttttcttcaccttcctctttgcctcatgtgtacacacacacacacatcttgtaccccagacctctgggtataattttcataattggtgcagaaagaagaaatgatctgaagatgtgttaaatggattgcaggggaaggaaggcccagggccctgtgtgtcatgccctcttgggttcctaagttctatgttccttagaggttctagcattaaacagataaagcccttcatggtcctggctgaggaagagtcttgctagggggattcagggaagacccgtgttaccagctcttaccctttatctggacagctctcctaccctgtatcttctcctcagatctgaggatagcaggctggactattggtgggcacctttcaagcccagggctactgtttgtcctgtggcagccggctacagtctcgtctgagtggcctcatctggacccttcctgttattaataaaacgcttctggaggccagatctgtgctcaagccatagttctgcttagaaagggatgccccacccttaccggacactgggaagaactgttggcccctagaaaccaaaggccaaactgaggctgccctgagttggaagaccactttctgaaatgcccatggactctgcctcccaaccattcgtctctcactcctagcagagctgtctgtgcagactgtttcttaggaggcacagcaagctccagggaaccctctgtgcttatgaagctcgtctggtgggcaaccccagcccactggacagagtcctcatggaaatgcctgggaagctgatttcatctaaggatgggttgaagtaggatgtgctcctgcgacttctcaggcaggtgagaggggtagtccttacactgtctagcataaacgccttccggaaggacctgcagctccagagaccacctcctgagcaccaagacctcttctggtggtgtggaaccagccaagagatttcaaggaagagtgattatttgatgaatgctatgggaatggcctcttctcttggagttctgaggcctggggatgcccaggaacactgggcacctgctgctgttagggccaatgcatagtctcagcaccggtgtcctaaggttaaggcggtgcgccttgtcatgtgctccttgtaccatgccatctgtgccagtgtgtgtctgcctcaccctgtgcttgacatgttcacccatcttctctgcttcccgccaccatccagatcctcagcggccgccccggctgtgcccttccctgctctcccgctctctcaggcctcggaaggaagatcggtggctgcgagctgaactaaggagtagggcctgtggctcagcgctaggccacgcacgcagcatcccaggcatgtggtgagaaactgccttaatgtgtctcctctgttcttgtcaacaggaggctcaagatgtgagaggtgtgagtcagacgcccgaggaacttacaggaggagcctaggtctgaagtcagtgttagggaagggcccatagccacttcctctgctcctgagcagggctgaagccgtttgcaagggacttgctttgcacagttttgctgtactttcacattttattatgtagcaagatacatggtgatttttttttttttcatttagcctgattttccaacgtcattggtgacaggccaaggccactatgttatttcctttgttctggtatccttcccttggaggaccttctctgagtagtggctccccaggtttgtcctttgagctgaggcaggaggctcacccattcttctgaataggaactgggtgttcccaccccccaaggactgcagggctttcccaagctgaggcaggaacgtgaggccagggaagagtgagcttcaccctcatcccacgctgtcctcctcaacccaccatgctcatcattctgtctcatccatccatccatccatccattcatcgccatgtgtccgcaagactgtctccatgaccctgaaaaaggactctcgagatgaaatcctttattcaaatgggacagcaagaaggaaaagccaatgtctggtgtctctccccccgcccctaccctgcgcgcatctatgtcttgtttggaatattgtctcttcaaccccctgttcatgtccttctcactcatgatcgatgtcttgtctgtgcactgtctctaacccaaatgcaaaggctgagtgtgaggtgatggccccgaggtccaggttgtagtcatggaaagagccctgctgtctcccttctcagggggcccattttagacacacaaagcccaaagaaaggtggtttgcaacagtgcttagctcgagcctccatatttccataactgttagcttaaaactgtggggttttaccttcctggaaccaaatgcattcttctgttgaggagtaacaggtctcaattcttttcaattaattttaaaagtcaatcactaagagcatcggctttgggccctgatgggcaggcatttccctggaaagggggtgaactacctacctctcctcaagacagccgaagggtgggattggtgccgctctgggaagcgtggccccaggagttttgtcctctgcagtttttaatgcaagttcactgccactttgacaaaagcccaattagaagccagtctctagttccttaaacaaaacagacagagtaaggaaaggaaggagggtggcagccagctggctggacactcgagaaagacggggaagtaagctacagaaagatagtcttcaaaaacaggtgtttgagagtgaatactctgtagaattgttagtggggtgtgtgtggtggtggtggggggatttctacaaaatagtcctttaagttgagtttacagcagatgaaaaatccaaccagcaaaattttgatcaaatttgaacaaaaacccaaaaacctaaaactgttgagcaggttgcgatgaggagcacagggctagctgcagagctggatcctcaggaggatagcgaattattttcaaccctggaatagaaaccacacactggcttgctgtgcaccagccactttgcatctaatccaagctttgaagggtgttgcttgggaggaaacaaatacagccttccatcttcactccagttagggatcctttcaaagtctccttcacagtgaggaaaaagagaagggtagaaactttagggagccggatttgtgtatcaattcctccgctgacagtcagtttctagatggagacagcctgcttaaagcaaatccgaatttaaataggacatttacatcggaaaagtctctccctaccttaatcccccattctcttgctttcaaaatacaagcacagcagtccttgaatggctgttgacccagggcacctagctgtccctgctggtcctggggctgccagaattcccttgggcgccaagcaacctgccaggtagccagtccctctgttacaagcctttgcatctggatagggaaaggggtggagacatacagtctgctttgtgttgaaacccagatttgtaccctgtgtttatacactgctgctggctcccgaggacagtgggactttagcaaggaagtgcagccgaggggtaaagagccctctggttcattgcctgatcggctttgagagagggtttggagggcaaggggctgcattcctctgagggacttggcctgaggcctttcgggcctctccagtgggttctgtttatcctctcatgggtgattatctcagtggtgtcaccaggggcttcctcccagaagtcagtcatccccaggccgtgcacccttttcagctggatgagagccagggatgcattctctccaaacagctaccctggcccattttaaggtaatctcattcttcaaaatgttccatagaatcctccaaattcccccagcagacttctaccctcgccaagttcccaaaacccactcagcaaagttgccaacctcgacgggctagcagtgtctaagcagcgatgggttcagtgttgtgtgtggtgaatactgtattttgtttcagttctgtctcccagataatgtgaaaacggtccaggagaaggcagcttcctatatgcagcgtgtgctttcttattcttatttttaatatatgacagttatttgagaagccatttctactttgaagtcattatcgatgaaagtgatgtatcttcacctaccattttcctaataaagttctgtattcaaatatagctgccaagcatcctcagtgaatgttaccatgtggaattttccacacttggttttaccccctcaaacctgactctgaccgtgcagtcttagcagaagagcttagcaggtcctagtgttcactcttggtctaactgctggtgtcagaagatctctacagggagaggtgttccattttctccacatgacctggattgctccttagaggtcagacagccttgcactgtacaaggcaatggcttagggtaaagtcccaggagttttccctacagtcccaagaatttggaagaggaaggcccacactacacatgcaggtcatggtggaaggtgacagaggaaggactctgtccctgtaagacagctggaaaccacaatattctgcatgttcctatcctgggtgaggacgctaatggaagtcaaaggggaatttgctaactgctgttggccagcttcctccaagaatcctgcttccccaacagacagagcctttgtctcttatagtttggtcttcagattctctttatcccacattcagccatttttgtaaaagagaggctagcaccagctccaaatatccaaatctgcagtgtttgagatctcactgcgcctcctccataccaacacatttgccattacttatagggtagttttcatgtgagttctaagttgattaacacacaagaattagaagggtgggaggctctaggaaaggcactgtgggactatttgactgcatgggtgtgaaaatgtaaggaacaggcaagagcttggatcccattctctctgcccacattgtgacttgagatatactaattgctcttgggggtctcagtcatataccatccataacagagttaaactgagagagatacaggatcagctagaatgaaaagcccaccccatgcttccagaaagtcccctctttatacctcctgtgatatgaactagaggaaaagcaattgactttgcttctcaaacagcctacggcaaagccctgtgagtttg

SEQ ID NO: 2 - DNA, 20 bp, Artificial sequence
  Primer
ccagagccaacgtcaagcat

SEQ ID NO: 3 - DNA, 20 bp, Artificial sequence
  Primer
cagccgtgcaacaatctgaa

SEQ ID NO: 4 - DNA, 29 bp, Artificial sequence
  Probe
tgaaaatcctcaacactccaaactgtgcc

SEQ ID NO: 5 - DNA, 16 bp, Artificial sequence
  Synthetic oligonucleotide
gcatgttctcacatta

SEQ ID NO: 6 - RNA, 20 nt, Artificial sequence
  Synthetic oligonucleotide
gauaaugugagaacaugccu

SEQ ID NO: 7 - DNA, 20 bp, Artificial sequence
  Primer
gccctcagaaagctctggaa

SEQ ID NO: 8 - DNA, 21 bp, Artificial sequence
  Primer
tagggtcgaggctctgcttgt

SEQ ID NO: 9 - DNA, 30 bp, Artificial sequence
  Probe
ccatcggtgcaaacctacagaagcagtatg

SEQ ID NO: 10 - DNA, 12 bp, Artificial sequence
  Synthetic oligonucleotide
tttttttttttt

SEQ ID NO: 11 - DNA, 12 bp, Artificial sequence
  Synthetic oligonucleotide
  misc_feature (11)..(12): bases at these positions are RNA
ttttttttttuu

(19) United States
(12) Patent Application Publication
(10) Pub. No.: US 2022/0064636 A1
(43) Pub. Date: Mar. 3, 2022

(54) MODIFIED OLIGOMERIC COMPOUNDS AND USES THEREOF

(71) Applicant: Ionis Pharmaceuticals, Inc., Carlsbad, CA (US)

(72) Inventors: Punit P. Seth, Carlsbad, CA (US); Michael T. Migawa, Carlsbad, CA (US); Graeme C. Freestone, La Jolla, CA (US)

(73) Assignee: Ionis Pharmaceuticals, Inc., Carlsbad, CA (US)

(21) Appl. No.: 17/282,335

(22) PCT Filed: Oct. 4, 2019
(86) PCT No.: PCT/US2019/054848

§ 371 Date: Apr. 1, 2021

Related U.S. Application Data
(60) Provisional application No. 62/887,547, filed on Aug. 15, 2019.
(60) Provisional application No. 62/742,265, filed on Oct. 5, 2018.
(60) Provisional application No. 62/746,511, filed on Oct. 16, 2018.

(30) Foreign Application Priority Data
Feb. 12, 2019 (US) PCT/US2019/017725

(51) Int. Cl. C12N 15/11; A61P 35/00

(52) U.S. Cl. CPC C12N 15/111; C12N 2310/3231; C12N 2310/315; A61P 35/00

(57) ABSTRACT

The present disclosure provides oligomeric compounds comprising a modified oligonucleotide having at least one stereo-non-standard nucleoside.

### SEQUENCE LISTING

The present application is being filed along with a Sequence Listing in electronic format. The Sequence Listing is provided as a file entitled CORE0155WOSEQ_ST25.txt created Oct. 3, 2019 which is 24 kb in size. The information in the electronic format of the sequence listing is incorporated herein by reference in its entirety.

### FIELD

The present disclosure provides oligomeric compounds comprising a modified oligonucleotide having at least one stereo-non-standard nucleoside.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics, therapeutic index, or affinity for a target nucleic acid.

### SUMMARY

In certain embodiments, the present disclosure provides oligomeric compounds comprising modified oligonucleotides having one or more stereo-non-stardard nucleosides. In certain embodiments, modified oligonucleotides having one or more stereo-non-stardard nucleosides show improved properties compared to similar modified oligonucleotides without one or more stereo-non-stardard nucleosides.

In certain embodiments, the present disclosure provides oligomeric compounds comprising a modified oligonucleotide consisting of 12-30 linked nucleosides, wherein at least one nucleoside of the modified oligonucleotide is a stereo-non-standard nucleoside having Formula I:

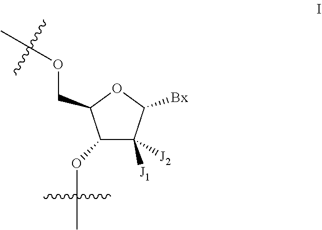

wherein one of J₁ and J₂ is H and the other of J₁ and J₂ is selected from H, OH, F, OCH₃, OCH₂CH₂OCH₃, O—C₁-C₆ alkoxy, and SCH₃; and wherein

Bx is a is a heterocyclic base moiety.

In certain embodiments, the present disclosure provides oligomeric compounds comprising a modified oligonucleotide consisting of 12-30 linked nucleosides, wherein at least one nucleoside of the modified oligonucleotide is a stereo-non-standard nucleoside having Formula II:

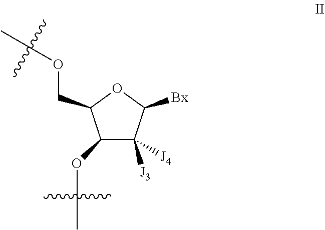

wherein one of J₃ and J₄ is H and the other of J₃ and J₄ is selected from H, OH, F, OCH₃, OCH₂CH₂OCH₃, O—C₁-C₆ alkoxy, and SCH₃; and wherein

Bx is a is a heterocyclic base moiety.

In certain embodiments, the present disclosure provides oligomeric compounds comprising a modified oligonucleotide consisting of 12-30 linked nucleosides, wherein at least one nucleoside of the modified oligonucleotide is a stereo-non-standard nucleoside having Formula III:

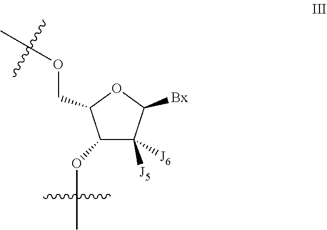

wherein one of J₅ and J₆ is H and the other of J₅ and J₆ is selected from H, OH, F, OCH₃, OCH₂CH₂OCH₃, O—C₁-C₆ alkoxy, and SCH₃; and wherein

Bx is a is a heterocyclic base moiety.

In certain embodiments, the present disclosure provides oligomeric compounds comprising a modified oligonucleotide consisting of 12-30 linked nucleosides, wherein at least one nucleoside of the modified oligonucleotide is a stereo-non-standard nucleoside having Formula IV:

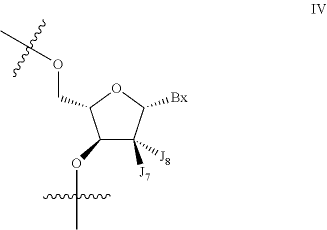

wherein one of J₇ and J₈ is H and the other of J₇ and J₈ is selected from H, OH, F, OCH₃, OCH₂CH₂OCH₃, O—C₁-C₆ alkoxy, and SCH₃; and wherein

Bx is a is a heterocyclic base moiety.

In certain embodiments, the present disclosure provides oligomeric compounds comprising a modified oligonucleotide consisting of 12-30 linked nucleosides, wherein at least one nucleoside of the modified oligonucleotide is a stereo-non-standard nucleoside having Formula V:

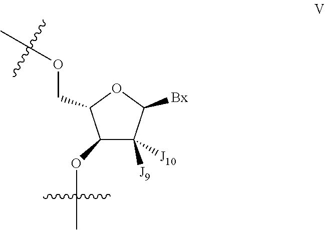

wherein one of J₉ and J₁₀ is H and the other of J₉ and J₁₀ is selected from H, OH, F, OCH₃, OCH₂CH₂OCH₃, O—C₁-C₆ alkoxy, and SCH₃; and wherein

Bx is a is a heterocyclic base moiety.

In certain embodiments, the present disclosure provides oligomeric compounds comprising a modified oligonucleotide consisting of 12-30 linked nucleosides, wherein at least one nucleoside of the modified oligonucleotide is a stereo-non-standard nucleoside having Formula VI:

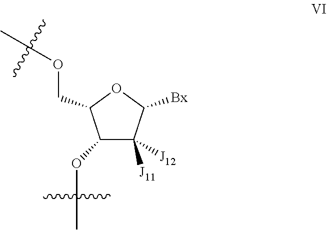

wherein one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein

Bx is a is a heterocyclic base moiety.

In certain embodiments, the present disclosure provides oligomeric compounds comprising a modified oligonucleotide consisting of 12-30 linked nucleosides, wherein at least one nucleoside of the modified oligonucleotide is a stereo-non-standard nucleoside having Formula VII:

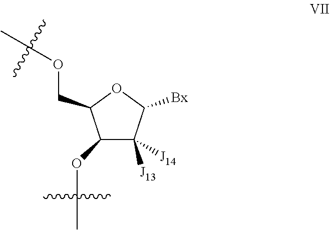

wherein one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and wherein

Bx is a is a heterocyclic base moiety.

In certain embodiments, the present disclosure provides a compound comprising a stereo-non-standard nucleoside having Formula VIII:

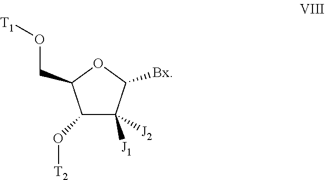

wherein one of J₁ or J₂ is H and the other of J₁ or J₂ is selected from OH, F, OCH₃, OCH₂CH₂OCH₃, O—C₁-C₆ alkoxy, and SCH₃;
T₁ is H or a hydroxyl protecting group;
T₂ is H, a hydroxyl protecting group, or a reactive phosphorus group; and wherein
Bx is a is a heterocyclic base moiety.

In certain embodiments, the present disclosure provides a compound comprising a stereo-non-standard nucleoside having Formula IX:

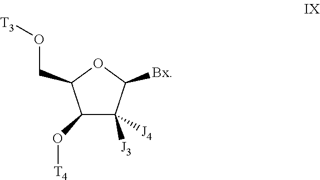

wherein one of J₃ or J₄ is H and the other of J₃ or J₄ is selected from H, OH, F, OCH₃, OCH₂CH₂OCH₃, O—C₁-C₆ alkoxy, and SCH₃;

T₃ is H or a hydroxyl protecting group;

T₄ is H, a hydroxyl protecting group, or a reactive phosphorus group; and wherein

Bx is a is a heterocyclic base moiety.

In certain embodiments, the present disclosure provides a compound comprising a stereo-non-standard nucleoside having Formula X:

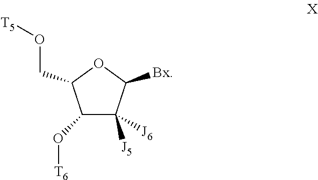

wherein one of J₅ or J₆ is H and the other of J₅ or J₆ is selected from H, OH, F, OCH₃, OCH₂CH₂OCH₃, O—C₁-C₆ alkoxy, and SCH₃;

T₅ is H or a hydroxyl protecting group;

T₆ is H, a hydroxyl protecting group, or a reactive phosphorus group; and wherein

Bx is a is a heterocyclic base moiety.

In certain embodiments, the present disclosure provides a compound comprising a stereo-non-standard nucleoside having Formula XI:

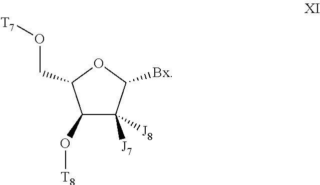

wherein one of J₇ or J₈ is H and the other of J₇ or J₈ is selected from OH, F, OCH₃, OCH₂CH₂OCH₃, O—C₁-C₆ alkoxy, and SCH₃,

T₇ is H or a hydroxyl protecting group;

T₈ is H, a hydroxyl protecting group, or a reactive phosphorus group; and wherein

Bx is a is a heterocyclic base moiety.

In certain embodiments, the present disclosure provides a compound comprising a stereo-non-standard nucleoside having Formula XII:

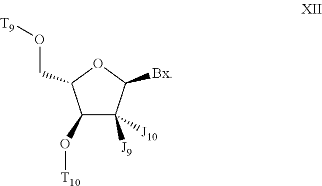

wherein one of J₉ or J₁₀ is H and the other of J₉ or J₁₀ is selected from OH, F, OCH₃, OCH₂CH₂OCH₃, O—C₁-C₆ alkoxy, and SCH₃;

T₉ is H or a hydroxyl protecting group;

T₁₀ is H, a hydroxyl protecting group, or a reactive phosphorus group; and wherein

Bx is a is a heterocyclic base moiety.

In certain embodiments, the present disclosure provides a compound comprising a stereo-non-standard nucleoside having Formula XIII:

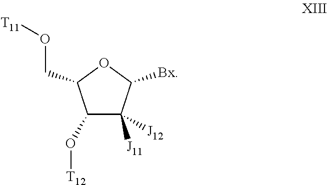

wherein one of J₁₁ or J₁₂ is H and the other of J₁₁ or J₁₂ is selected from H, OH, F, OCH₃, OCH₂CH₂OCH₃, O—C₁-C₆ alkoxy, and SCH₃;

T₁₁ is H or a hydroxyl protecting group;

T₁₂ is H, a hydroxyl protecting group, or a reactive phosphorus group; and wherein

Bx is a is a heterocyclic base moiety.

In certain embodiments, the present disclosure provides a compound comprising a stereo-non-standard nucleoside having Formula XIV:

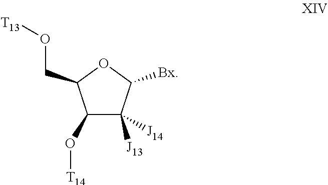

wherein one of J₁₃ or J₁₄ is H and the other of J₁₃ or J₁₄ is selected from H, OH, F, OCH₃, OCH₂CH₂OCH₃, O—C₁-C₆ alkoxy, and SCH₃;

T₁₃ is H or a hydroxyl protecting group;

T₁₄ is H, a hydroxyl protecting group, or a reactive phosphorus group; and wherein

Bx is a is a heterocyclic base moiety.

In certain embodiments, the modified oligonucleotides having at least one stereo-non-standard nucleoside have an increased maximum tolerated dose when administered to an animal compared to an otherwise identical oligomeric compound, except that the otherwise identical oligomeric compound lacks the at least one stereo-non-standard nucleoside.

In certain embodiments, the modified oligonucleotides having at least one stereo-non-standard nucleoside have an increased therapeutic index compared to an otherwise identical oligomeric compound, except that the otherwise identical oligomeric compound lacks the at least one stereo-non-standard nucleoside.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH(H) sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “4′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 4′-position and is a non-bicyclic furanosyl sugar moiety. 4′-substituted furanosyl sugar moieties do not comprise additional substituents at other positions of the furanosyl sugar moiety other than a nucleobase and/or internucleoside linkage(s) when in the context of an oligonucleotide.

As used herein, “5′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 5′-position and is a non-bicyclic furanosyl sugar moiety. 5′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to routes of introducing a compound or composition provided herein to a subject. Examples of routes of administration that can be used include, but are not limited to, administration by inhalation, subcutaneous injection, intrathecal injection, and oral administration.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified bicyclic furanosyl sugar moiety. In certain embodiments, the bicyclic sugar moiety does not comprise a furanosyl moiety.

As used herein, “cEt” or “constrained ethyl” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the β-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups may comprise a conjugate moiety and a conjugate linker that attaches the conjugate moiety to the oligonucleotide.

As used herein, “conjugate linker” means a bond or a group of atoms comprising at least one bond that connects a conjugate moiety to an oligonucleotide.

As used herein, “conjugate moiety” means a group of atoms that is attached to an oligonucleotide via a conjugate linker.

As used herein, “cytotoxic” or “cytotoxicity” in the context of an effect of an oligomeric compound or a parent oligomeric compound on cultured cells means an at least 2-fold increase in caspase activation following administration of 10 μM or less of the oligomeric compound or parent oligomeric compound to the cultured cells relative to cells cultured under the same conditions but that are not administered the oligomeric compound or parent oligomeric compound. In certain embodiments, cytotoxicity is measured using a standard in vitro cytotoxicity assay.

As used herein, “deoxy region” means a region of 5-12 contiguous nucleotides, wherein at least 70% of the nucleosides are stereo-standard DNA nucleosides. In certain embodiments, each nucleoside is selected from a stereo-standard DNA nucleoside (a nucleoside comprising a β-D-2′-deoxyribosyl sugar moiety), a stereo-non-standard nucleoside of Formula I-VII, a bicyclic nucleoside, and a substituted stereo-standard nucleoside. In certain embodiments, a deoxy region supports RNase H activity. In certain embodiments, a deoxy region is the gap of a gapmer.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region comprising a plurality of nucleosides that support RNase H cleavage positioned between a 5′-region and a 3′-region. In certain embodiments, the nucleosides of the 5′-region and 3′-region each comprise a 2′-substituted furanosyl sugar moiety or a bicyclic sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”. Gaps of gapmers are deoxy regions.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, the terms “internucleoside linkage” means a group of atoms or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that does not have a negatively charged phosphate in a buffered aqueous solution at pH=7.0.

As used herein, “abasic nucleoside” means a sugar moiety in an oligonucleotide or oligomeric compound that is not directly connected to a nucleobase. In certain embodiments, an abasic nucleoside is adjacent to one or two nucleosides in an oligonucleotide.

As used herein, “linked nucleosides” are nucleosides that are connected in a continuous sequence (i.e. no additional nucleosides are present between those that are linked).

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay, e.g. the assay of Example 4.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “MOE” means methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (NC) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of (1) an oligonucleotide (a single-stranded oligomeric compound) or two oligonucleotides hybridized to one another (a double-stranded oligomeric compound); and (2) optionally one or more additional features, such as a conjugate group or terminal group which may be bound to the oligonucleotide of a single-stranded oligomeric compound or to one or both oligonucleotides of a double-stranded oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 12-30 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, the term “single-stranded” in reference to an antisense compound means such a compound consists of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “stereo-standard nucleoside” means a nucleoside comprising a non-bicyclic furanosyl sugar moiety having the configuration of naturally occurring DNA and RNA as shown below. A “stereo-standard DNA nucleoside” is a nucleoside comprising a β-D-2′-deoxyribosyl sugar moiety. A “stereo-standard RNA nucleoside” is a nucleoside comprising a β-D-ribosyl sugar moiety. A “substituted stereo-standard nucleoside” is a stereo-standard nucleoside other than a stereo-standard DNA or stereo-standard RNA nucleoside. In certain embodiments, R₁ is a 2′-substituent and R₂-R₅ are each H. In certain embodiments, the 2′-substituent is selected from OMe, F, OCH₂CH₂OCH₃, O-alkyl, SMe, or NMA. In certain embodiments, R₁-R₄ are H and R₅ is a 5′-substituent selected from methyl, allyl, or ethyl. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methyl cytosine, adenine or guanine.

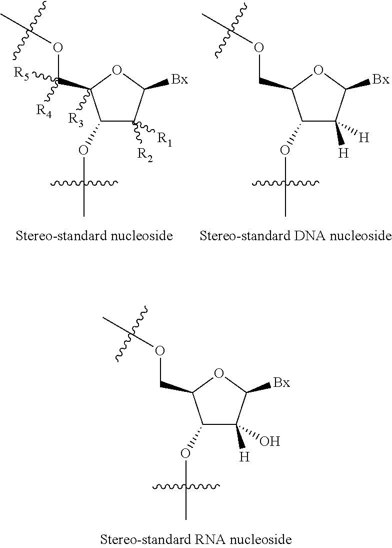

As used herein, “stereo-non-standard nucleoside” means a nucleoside comprising a non-bicyclic furanosyl sugar moiety having a configuration other than that of a stereo-standard sugar moiety. In certain embodiments, a “stereo-non-standard nucleoside” is represented by Formulas I-VII below. In certain embodiments, J₁-J₁₄ are independently selected from H, OH, F, OCH₃, OCH₂CH₂OCH₃, O—C₁-C₆ alkoxy, and SCH₃. A “stereo-non-standard RNA nucleoside” has one of formulas I-VII below, wherein each of J₁, J₃, J₅, J₇, J₉, J₁₁, and J₁₃ is H, and each of J₂, J₄, J₆, J₈, J₁₀, J₁₂, and J₁₄ is OH. A “stereo-non-standard DNA nucleoside” has one of formulas I-VII below, wherein each J is H. A “2′-substituted stereo-non-standard nucleoside” has one of formulas I-VII below, wherein either J₁, J₃, J₅, J₇, J₉, J₁₁, and J₁₃ is other than H and/or or J₂, J₄, J₆, J₈, J₁₀, J₁₂, and J₁₄ is other than H or OH. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methyl cytosine, adenine or guanine.

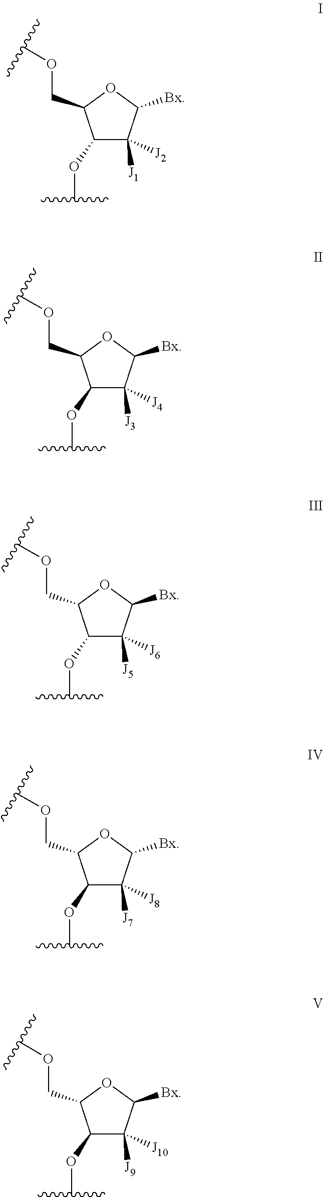

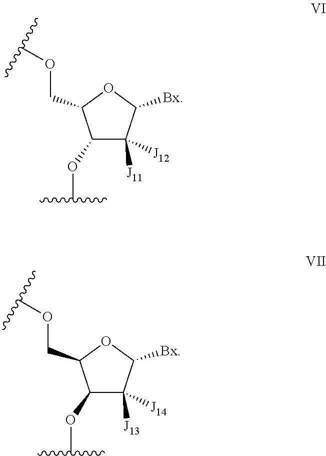

As used herein, “stereo-standard sugar moiety” means the sugar moiety of a stereo-standard nucleoside.

As used herein, “stereo-non-standard sugar moiety” means the sugar moiety of a stereo-non-standard nucleoside.

As used herein, “substituted stereo-non-standard nucleoside” means a stereo-non-standard nucleoside comprising a substituent other than the substituent corresponding to natural RNA or DNA. Substituted stero-non-standard nucleosides include but are not limited to nucleosides of Formula I-VII wherein the J groups are other than: (1) both H or (2) one H and the other OH.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may be stereo-non-standard sugar moieties. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

As used herein, “treat” refers to administering a compound or pharmaceutical composition to an animal in order to effect an alteration or improvement of a disease, disorder, or condition in the animal.

### Certain Compounds

In certain embodiments, compounds described herein are oligomeric compounds comprising or consisting of oligonucleotides consisting of linked nucleosides and having at least one stereo-non standard nucleoside. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety, a stereo-non-stardard nucleoside, and/or a modified nucleobase) and/or at least one modified internucleoside linkage).

I. Modifications

A. Modified Nucleosides

Modified nucleosides comprise a stereo-non-stardard nucleoside, or a modified sugar moiety, or a modified nucleobase, or any combination thereof

1. Certain Modified Sugar Moieties

In certain embodiments, modified sugar moieties are stereo-non-stardard sugar moieties. In certain embodiments, sugar moieties are substituted furanosyl stereo-standard sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

a. Stereo-Non-Standard Sugar Moieties

In certain embodiments, modified sugar moieties are stereo-non-standard sugar moieties shown in Formula I:

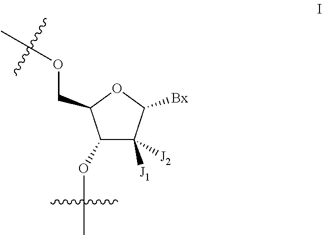

wherein one of J₁ and J₂ is H and the other of J₁ and J₂ is selected from H, OH, F, OCH₃, OCH₂CH₂OCH₃, O—C₁-C₆ alkoxy, and SCH₃; and wherein

Bx is a is a heterocyclic base moiety.

In certain embodiments, modified sugar moieties are stereo-non-standard sugar moieties shown in Formula II:

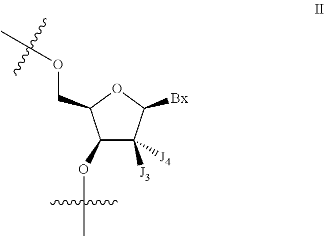

wherein one of J₃ and J₄ is H and the other of J₃ and J₄ is selected from H, OH, F, OCH₃, OCH₂CH₂OCH₃, O—C₁-C₆ alkoxy, and SCH₃; and wherein

Bx is a is a heterocyclic base moiety.

In certain embodiments, modified sugar moieties are stereo-non-standard sugar moieties shown in Formula III:

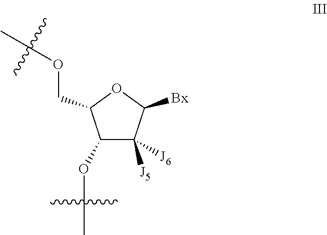

wherein one of J₅ and J₆ is H and the other of J₅ and J₆ is selected from H, OH, F, OCH₃, OCH₂CH₂OCH₃, O—C₁-C₆ alkoxy, and SCH₃; and wherein

Bx is a is a heterocyclic base moiety.

In certain embodiments, modified sugar moieties are stereo-non-standard sugar moieties shown in Formula IV:

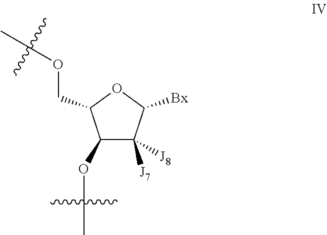

wherein one of J₇ and J₈ is H and the other of J₇ and J₈ is selected from H, OH, F, OCH₃, OCH₂CH₂OCH₃, O—C₁-C₆ alkoxy, and SCH₃; and wherein

Bx is a is a heterocyclic base moiety.

In certain embodiments, modified sugar moieties are stereo-non-standard sugar moieties shown in Formula V:

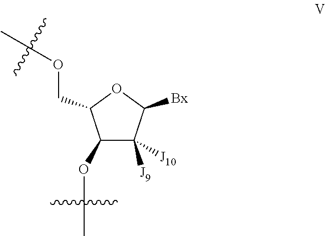

wherein one of J₉ and J₁₀ is H and the other of J₉ and J₁₀ is selected from H, OH, F, OCH₃, OCH₂CH₂OCH₃, O—C₁-C₆ alkoxy, and SCH₃; and wherein

Bx is a is a heterocyclic base moiety.

In certain embodiments, modified sugar moieties are stereo-non-standard sugar moieties shown in Formula VI:

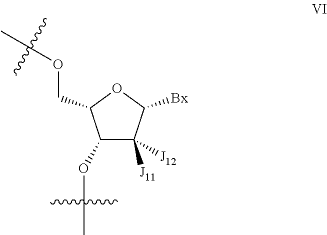

wherein one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein

Bx is a is a heterocyclic base moiety.

In certain embodiments, modified sugar moieties are stereo-non-standard sugar moieties shown in Formula VII:

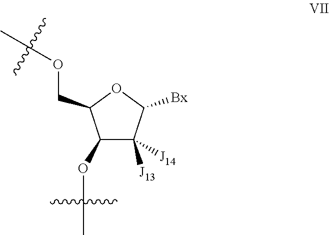

wherein one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and wherein

Bx is a is a heterocyclic base moiety.

b. Substituted Stereo-Standard Sugar Moieties

In certain embodiments, modified sugar moieties are substituted stereo-standard furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments one or more acyclic substituent of substituted stereo-standard sugar moieties is branched. Examples of 2′-substituent groups suitable for substituted stereo-standard sugar moieties include but are not limited to: 2′-F, 2′-OCH₃ (“2′-OMe” or “2′-O-methyl”), and 2′-O(CH₂)₂OCH₃ (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for substituted stereo-standard sugar moieties include but are not limited to alkoxy (e.g., methoxy), alkyl, and those described in Manoharan et al., WO 2015/106128. Examples of 5′-substituent groups suitable for substituted stereo-standard sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′,4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83: 9839-9849.

In certain embodiments, a 2′-substituted stereo-standard nucleoside comprises a sugar moiety comprising a non-bridging 2′-substituent group selected from: F, NH₂, N₃, OCF₃, OCH₃, SCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted stereo-standard nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”). In certain embodiments, a 2′-substituted stereo-standard nucleoside comprises a sugar moiety comprising a 2′-substituent group selected from: F, OCH₃, and OCH₂CH₂OCH₃.

In certain embodiments, the 4′ 0 of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71(3): 921-925 (4′-S-fluoro5-methylarauridine or FAMU).

c. Bicyclic Nucleosides

Certain nucleosides comprise modified sugar moieties that comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672), 4′-C(═O)—N(CH₃)₂-2′, 4′-C(═O)—N(R)₂-2′, 4′-C(═S)—N(R)₂-2′ and analgos thereof (see, e.g., Obika et al., WO2011052436A1, Yusuke, WO2017018360A1).

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.; Christiansen, et al., J. Am. Chem. Soc. 1998, 120, 5458-5463; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

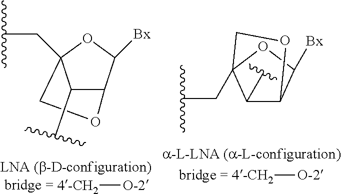

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

The term “substituted” following a position of the furanosyl ring, such as “2′-substituted” or “2′-4′-substituted”, indicates that is the only position(s) having a substituent other than those found in unmodified sugar moieties in oligonucleotides.

d. Sugar Surrogates

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, C J. Bioorg. & Med. Chem. 2002, 10, 841-854), fluoro HNA (“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran).

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

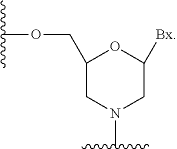

In certain embodiments, morpholinos may be modified, for example by adding or altering various substituent groups from the above morpholino structure. Such sugar surrogates are referred to herein as “modified morpholinos.” In certain embodiments, morpholino residues replace a full nucleotide, including the internucleoside linkage, and have the structures shown below, wherein Bx is a heterocyclic base moiety.

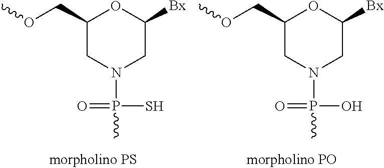

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA”, see Schlegel, et al., J. Am. Chem. Soc. 2017, 139:8537-8546) and nucleosides and oligonucleotides described in Manoharan et al., WO2011/133876.

Many other bicyclic and tricyclic sugar and sugar surrogate ring systems are known in the art that can be used in modified nucleosides. Certain such ring systems are described in Hanessian, et al., J. Org. Chem., 2013, 78: 9051-9063 and include bcDNA and tcDNA. Modifications to bcDNA and tcDNA, such as 6′-fluoro, have also been described (Dogovic and Leumann, J. Org. Chem., 2014, 79: 1271-1279).

2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinaset al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to an target nucleic acid comprising one or more modified nucleobases. In certain embodiments, the modified nucleobase is 5-methylcytosine. In certain embodiments, each cytosine is a 5-methylcytosine.

B. Modified Internucleoside Linkages

In certain embodiments, compounds described herein having one or more modified internucleoside linkages are selected over compounds having only phosphodiester internucleoside linkages because of desirable properties such as, for example, enhanced cellular uptake, enhanced affinity for target nucleic acids, and increased stability in the presence of nucleases.

In certain embodiments, compounds comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

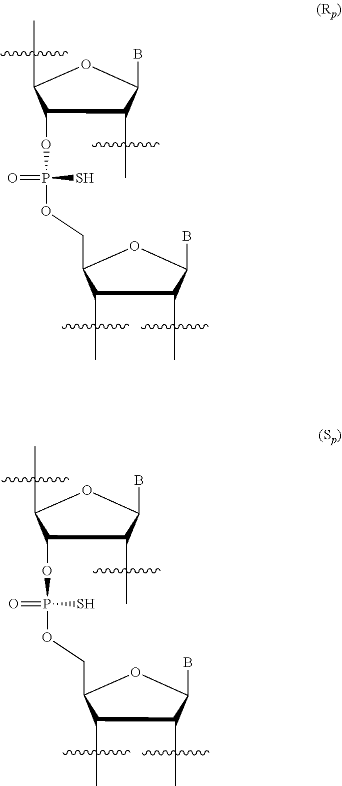

Unless otherwise indicated, chiral internucleoside linkages of modified oligonucleotides described herein can be stereorandom or in a particular stereochemical configuration.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′- O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, nucleic acids can be linked 2′ to 5′ rather than the standard 3′ to 5′ linkage. Such a linkage is illustrated below.

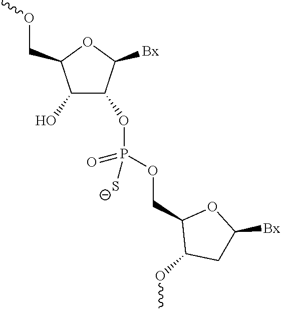

In certain embodiments, nucleosides can be linked by vinicinal 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown below.

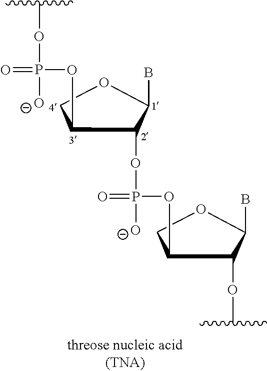

Additional modified linkages include α,β-D-CNA type linkages and related conformationally-constrained linkages, shown below, Synthesis of such molecules has been described previously (see Dupouy, et al., Angew. Chem. Int. Ed. Engl., 2014, 45: 3623-3627; Borsting, et al. Tetahedron, 2004, 60:10955-10966; Ostergaard, et al. ACS Chem. Biol. 2014, 9: 1975-1979; Dupouy, et al., Eur. J. Org. Chem., 2008, 1285-1294; Martinez, et al., PLoS One, 2011, 6:e25510; Dupouy, et al., Eur. J. Org. Chem., 2007, 5256-5264; Boissonnet, et. al., New J. Chem., 2011, 35: 1528-1533.)

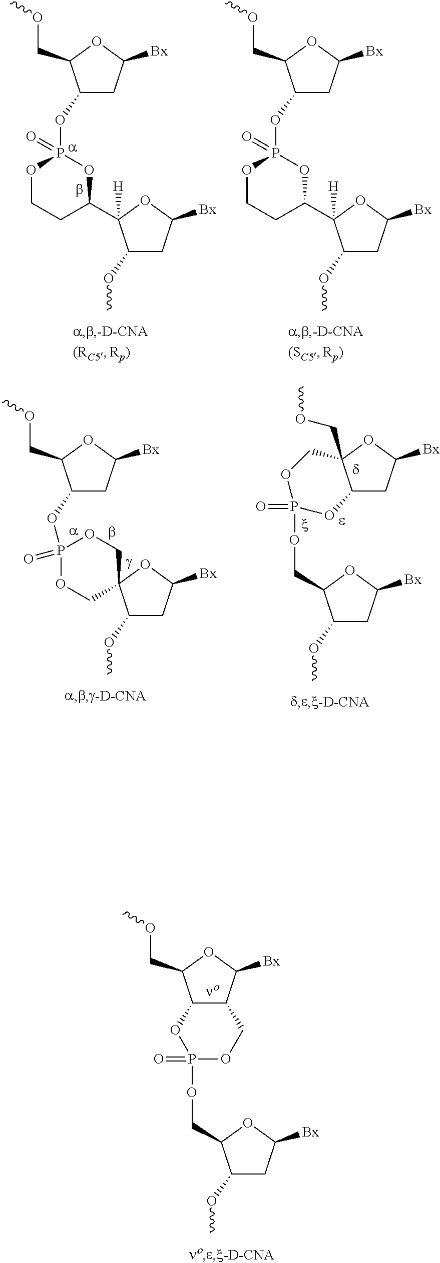

II. Certain Motifs

In certain embodiments, oligomeric compounds described herein comprise or consist of oligonucleotides. Modified oligonucleotides can be described by their motif, e.g. a pattern of unmodified and/or modified sugar moieties, nucleobases, and/or internucleoside linkages. In certain embodiments, modified oligonucleotides comprise one or more stereo-non-standard nucleosides. In certain embodiments, modified oligonucleotides comprise one or more stereo-standard nucleosides. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

A. Certain Sugar Motifs

In certain embodiments, oligomeric compounds described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include without limitation any of the sugar modifications discussed herein.

In certain embodiments, a modified oligonucleotide comprises or consists of a gapmer. The sugar motif of a gapmer defines the regions of the gapmer: 5′-region, central region (gap), and 3′-region. The central region is linked directly to the 5′-region and to the 3′-region with no nucleosides intervening. The central region is a deoxy region. The nucleoside at the first position (position 1) from the 5′-end of the central region and the nucleoside at the last position of the central region are adjacent to the 5′-region and 3′-region, respectively, and each comprise a sugar moiety independently selected from a 2′-deoxyfuranosyl sugar moiety or a sugar surrogate. In certain embodiments, the nucleoside at position 1 of the central region and the nucleoside at the last position of the central region are DNA nucleosides, selected from stereo-standard DNA nucleosides or stereo-non-standard DNA nucleosides having any of Formulas I-VII, wherein each J is H. In certain embodiments, the nucleoside at the first and last positions of the central region adjacent to the 5′ and 3′ regions are stereo-standard DNA nucleosides. Unlike the nucleosides at the first and last positions of the central region, the nucleosides at the other positions within the central region may comprise a 2′-substituted stereo-standard sugar moiety or a substituted stereo-non-standard sugar moiety or a bicyclic sugar moiety. In certain embodiments, each nucleoside within the central region supports RNase H cleavage. In certain embodiments, a plurality of nucleosides within the central region support RNase H cleavage.

In certain embodiments, the central region comprises at least one stereo-non-standard nucleoside selected from Formula I-VII. In certain embodiments, the central region comprises at least two, at least three, at least four, at least five, or at least six stereo-non-standard nucleosides selected from Formula I-VH. In certain embodiments, the central region comprises exactly one stereo-non-standard nucleoside. In certain embodiments, the central region comprises exactly two stereo-non-standard nucleosides. In certain embodiments, the central region comprises exactly three stereo-non-standard nucleosides. In certain embodiments, the central region comprises exactly four stereo-non-standard nucleosides. In certain embodiments, the central region comprises exactly five stereo-non-standard nucleosides. In certain embodiments, the central region comprises exactly 6, 7, 8, 9, or 10 stereo-non-standard nucleosides. In certain embodiments, the remainder of the nucleosides of the central region are stereo-standard DNA nucleosides. In certain embodiments, exactly one nucleoside of the central region is a 2′-substituted stereo-non-standard nucleoside, and the remainder of the nucleosides of the central region are stereo-standard DNA nucleosides. In certain embodiments, exactly one nucleoside of the central region is a 2′-OMe stereo-non-standard nucleoside, and the remainder of the nucleosides of the central region are stereo-standard DNA nucleosides. In certain embodiments, one or more nucleosides of the central region is a stereo-non-standard nucleoside, the nucleoside at position 2 of the central region is a stereo-standard 2′-OMe nucleoside, and the remainder of the nucleosides of the central region are stereo-standard DNA nucleosides. In certain embodiments, each nucleoside of the central region is a stereo-non-standard nucleoside.

In certain embodiments, the nucleoside at the first position of the central region is a stereo-non-standard DNA nucleoside. In certain embodiments, the nucleoside at the last position of the central region is a stereo-non-standard DNA nucleoside.

In certain embodiments, the nucleoside at the second position of the central region is a stereo-non-standard nucleoside. In certain embodiments, the nucleoside at the third position of the central region is a stereo-non-standard nucleoside. In certain embodiments, the nucleoside at the fourth position of the central region is a stereo-non-standard nucleoside. In certain embodiments, the nucleoside at the fifth position of the central region is a stereo-non-standard nucleoside. In certain embodiments, the nucleoside at the sixth position of the central region is a stereo-non-standard nucleoside. In certain embodiments, the nucleoside at the seventh position of the central region is a stereo-non-standard nucleoside. In certain embodiments, the nucleoside at the eighth position of the central region is a stereo-non-standard nucleoside. In certain embodiments, the nucleoside at the ninth position of the central region is a stereo-non-standard nucleoside. In certain embodiments, the nucleoside at the tenth position of the central region is a stereo-non-standard nucleoside. In any of such embodiments, the stereo-non-standard nucleoside may be a substituted stereo-non-standard nucleoside.

The 3′-most nucleoside of the 5′-region and the 5′-most nucleoside of the 3′-region are substituted stereo-standard nucleosides or bicyclic nucleosides. In certain embodiments, each nucleoside of the 5′-region and the 3′-region is either a stereo-standard nucleoside or a bicyclic nucleoside. In certain embodiments, each nucleoside of the 5′-region and the 3′-region is either a substituted stereo-standard nucleoside or a bicyclic nucleoside. In certain embodiments, the bicyclic sugar moiety in the 5′ and 3′-regions is a 4′-2′-bicyclic sugar moiety. In certain embodiments, the bicyclic sugar moiety in the 5′ and 3′ regions is a cEt. In certain embodiments, the stereo-standard sugar moiety is a 2′-MOE-β-D-ribofuranosyl sugar moiety.

Herein, the lengths (number of nucleosides) of the three regions of a gapmer may be provided using the notation [# of nucleosides in the 5′-region]−[# of nucleosides in the central region]−[# of nucleosides in the 3′-region]. Thus, a 3-10-3 gapmer consists of 3 linked nucleosides in each of the 3′ and 5′ regions and 10 linked nucleosides in the central region. Where such nomenclature is followed by a specific modification, that modification is the modification of each sugar moiety of each 5′ and 3′-region and the central region nucleosides comprise stereo-standard DNA sugar moieties. Thus, a 5-10-5 MOE gapmer consists of 5 linked nucleosides each comprising 2′-MOE-stereo-standard sugar moieties in the 5′-region, 10 linked nucleosides each comprising a stereo-standard DNA sugar moiety in the central region, and 5 linked nucleosides each comprising 2′-MOE-stereo-standard sugar moieties in the 3′-region. A 5-10-5 MOE gapmer having a substituted stereo-non-standard nucleoside at position 2 of the gap has a gap of 10 nucleosides wherein the 2^{nd} nucleoside of the gap is a substituted stereo-non-standard nucleoside rather than the stereo-standard DNA nucleoside. Such oligonucleotide may also be described as a 5-1-1-8-5 MOE/substituted stereo-non-standard/MOE gapmer. A 3-10-3 cEt gapmer consists of 3 linked nucleosides each comprising a cEt in the 5′-region, 10 linked nucleosides each comprising a stereo-standard DNA sugar moiety in the central region, and 3 linked nucleosides each comprising a cEt in the 3′-region. A 3-10-3 cEt gapmer having a substituted stereo-non-standard nucleoside at position 2 of the gap has a gap of 10 nucleoside wherein the 2^{nd} nucleoside of the gap is a substituted stereo-non-standard nucleoside rather than the stereo-standard DNA nucleoside. Such oligonucleotide may also be described as a 3-1-1-8-3 cEt/substituted stereo-non-standard/cEt gapmer.

The sugar motif of a gapmer may also be denoted by a notation where different letters indicate various nucleosides. For example: kkk-dx*d(8)-kkk, wherein each “k” represents a cEt nucleoside, each “d” represents a stereo standard DNA and x* represents a substituted stereo-non-standard nucleoside. Certain MOE gapmers may be denoted by the following notations eeeee-dx*(8)-eeeee or e(5)-dx*(8)-e(5), wherein each “e” represents a 2′-MOE-stereo standard nucleosides, each “d” represents a stereo standard DNA, and each x* represents a substituted stereo-non-standard nucleoside. Sugar motifs are independent of the nucleobase sequence, the internucleoside linkage motif, and any nucleobase modifications.

B. Certain Nucleobase Motifs

In certain embodiments, oligomeric compounds described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

C. Certain Internucleoside Linkage Motifs

In certain embodiments, oligomeric compounds described herein comprise or consist of oligonucleotides. In certain embodiments, oligonucleotides comprise modified and/or unmodified internucleoside linkages arranged along the oligonucleotide or region thereof in a defined pattern or motif. In certain embodiments, each internucleoside linkage is a phosphodiester internucleoside linkage (P═O). In certain embodiments, each internucleoside linkage of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the internucleoside linkages within the central region of a modified oligonucleotide are all modified. In certain such embodiments, some or all of the internucleoside linkages in the 5′-region and 3′-region are unmodified phosphate linkages. In certain embodiments, the terminal internucleoside linkages are modified. In certain embodiments, the internucleoside linkage motif comprises at least one phosphodiester internucleoside linkage in at least one of the 5′-region and the 3′-region, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such internucleoside linkage motifs.

In certain embodiments, oligonucleotides comprise a region having an alternating internucleoside linkage motif. In certain embodiments, oligonucleotides comprise a region of uniformly modified internucleoside linkages. In certain such embodiments, the internucleoside linkages are phosphorothioate internucleoside linkages. In certain embodiments, all of the internucleoside linkages of the oligonucleotide are phosphorothioate internucleoside linkages. In certain embodiments, each internucleoside linkage of the oligonucleotide is selected from phosphodiester or phosphate and phosphorothioate. In certain embodiments, each internucleoside linkage of the oligonucleotide is selected from phosphodiester or phosphate and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, modified oligonucleotides comprise a linkage motif comprising all phosphorothioate linkages except for one or two methylphosphonate linkages. In certain embodiments, one methylphosphonate linkage is in the central region of an oligonucleotide.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

III. Certain Modified Oligonucleotides

In certain embodiments, oligomeric compounds described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar moiety, region or segment B consists of 6-10 linked nucleosides having a specified sugar moiety, and region or segment C consists of 2-6 linked nucleosides having a specified sugar moiety. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence. In certain embodiments, when a DNA nucleoside or DNA-like nucleoside that comprises a T in a DNA sequence is replaced with a RNA-like nucleoside, the nucleobase T is replaced with the nucleobase U. Each of these compounds has an identical target RNA.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

IV. Certain Conjugated Compounds

In certain embodiments, the oligomeric compounds described herein comprise or consist of an oligonucleotide (modified or unmodified) and optionally one or more conjugate groups and/or terminal groups. Conjugate groups consist of one or more conjugate moiety and a conjugate linker that links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, a conjugate linker is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an oligomeric compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the compound has been taken up, it is desirable that the conjugate group be cleaved to release the unconjugated oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

3. Certain Cell-Targeting Conjugate Moieties

In certain embodiments, a conjugate group comprises a cell-targeting conjugate moiety. In certain embodiments, a conjugate group has the general formula:

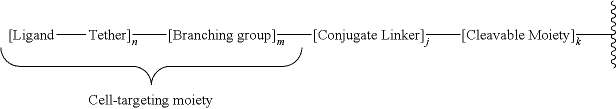

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Oligomeric compounds described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds or a salt thereof. In certain embodiments, the oligomeric compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

In certain embodiments, oligomeric compounds described herein comprise or consist of modified oligonucleotides having at least one stereo-non-standard nucleoside. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in in the RNA:DNA duplex is tolerated.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Certain Oligomeric Compounds

In certain embodiments, oligomeric compounds described herein having one or more stereo-non-standard nucleosides are selected over compounds lacking such stereo-non-standard nucleosides because of one or more desirable properties. In certain embodiments, oligomeric compounds described herein having one or more stereo-non-standard nucleosides have enhanced cellular uptake. In certain embodiments, oligomeric compounds described herein having one or more stereo-non-standard nucleosides have enhanced bioavailability. In certain embodiments, oligomeric compounds described herein having one or more stereo-non-standard nucleosides have enhanced affinity for target nucleic acids. In certain embodiments, oligomeric compounds described herein having one or more stereo-non-standard nucleosides have increased stability in the presence of nucleases. In certain embodiments, oligomeric compounds described herein having one or more stereo-non-standard nucleosides have increased interactions with certain proteins. In certain embodiments, oligomeric compounds described herein having one or more stereo-non-standard nucleosides have decreased interactions with certain proteins. In certain embodiments, oligomeric compounds described herein having one or more stereo-non-standard nucleosides have increased RNase H activity. In certain embodiments, incorporation of one or more stereo-non-standard nucleosides into a modified oligonucleotide within the central region can significantly reduce toxicity with only a modest loss in potency, if any. In certain embodiments, incorporation of one or more stereo-non-standard nucleosides into a modified oligonucleotide at positions 2, 3 or 4 of the central region can significantly reduce toxicity with only a modest loss in potency, if any. In certain embodiments, incorporation of one or more stereo-non-standard nucleosides into a modified oligonucleotide at position 2 of the central region can significantly reduce toxicity with only a modest loss in potency, if any. In certain such embodiments, the stereo-non-standard nucleoside is a stereo-non-standard nucleoside of Formula I, Formula II, Formula III, Formula IV, Formula V, Formula VI, or Formula VII.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron.

### Certain Compounds

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or 13 such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples are intended to illustrate certain aspects of the invention and are not intended to limit the invention in any way.

### Example 1: Design and Activity of Modified Oligonucleotides with 2′-Substituted Stereo-Standard Nucleosides and 2′-Substituted Stereo-Non-Standard Nucleosides

As described in Table 1, below modified oligonucleotides having either 2′-substituted stereo standard nucleosides or 2′-substituted stereo non-standard nucleosides in the gap were synthesized using standard techniques or those described herein. The modified oligonucleotides were compared to compound 558807, which is a 3-10-3 cEt gapmer, having uniform phosphorothioate (P═S) internucleoside linkages throughout the compound.

TABLE 1
Design and activity of modified oligonucleotides containing 2′-substituted
stereo-standard nucleosides and 2′-substituted stereo-non-standard nucleosides

| Compound | | IC50 | SEQ ID |
|---|---|---|---|
| Number | Chemistry Notation (5′-3′) | (nM) | NO. |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 67 | 5 |
| 1385844 | Gₖₛ^{m}CₖₛAₖₛTₘ₂ₛG_{ds}T_{ds}T_{ds}^{m}C_{ds}T_{ds}^{m}C_{ds}A_{ds}^{m}C_{ds}A_{ds}TₖₛTₖₛAₖ | 101 | 5 |
| 1385840 | Gₖₛ^{m}CₖₛAₖₛT_{ds}G_{ds}Tₘ₂ₛT_{ds}^{m}C_{ds}T_{ds}^{m}C_{ds}A_{ds}^{m}C_{ds}A_{ds}TₖₛTₖₛAₖ | 135 | 5 |
| 1385841 | Gₖₛ^{m}CₖₛAₖₛT_{ds}G_{ds}T_{ds}Tₘ₂ₛ^{m}C_{ds}T_{ds}^{m}C_{ds}A_{ds}^{m}C_{ds}A_{ds}TₖₛTₖₛAₖ | 162 | 5 |
| 1385845 | Gₖₛ^{m}CₖₛAₖₛT_{ds}G_{ds}T_{ds}T_{ds}^{m}C_{ds}Tₘ₂ₛ^{m}C_{ds}A_{ds}^{m}C_{ds}A_{ds}TₖₛTₖₛAₖ | 93 | 5 |
| 1385842 | Gₖₛ^{m}CₖₛAₖₛ[_{α-L}Tₘₛ]G_{ds}T_{ds}T_{ds}^{m}C_{ds}T_{ds}^{m}C_{ds}A_{ds}^{m}C_{ds}A_{ds}TₖₛTₖₛAₖ | 114 | 5 |
| 1385838 | Gₖₛ^{m}CₖₛAₖₛT_{ds}G_{ds}[_{α-L}Tₘₛ]T_{ds}^{m}C_{ds}T_{ds}^{m}C_{ds}A_{ds}^{m}C_{ds}A_{ds}TₖₛTₖₛAₖ | 111 | 5 |
| 1385839 | Gₖₛ^{m}CₖₛAₖₛT_{ds}G_{ds}T_{ds}[_{α-L}Tₘₛ]^{m}C_{ds}T_{ds}^{m}C_{ds}A_{ds}^{m}C_{ds}A_{ds}TₖₛTₖₛAₖ | 262 | 5 |
| 1385843 | Gₖₛ^{m}CₖₛAₖₛT_{ds}G_{ds}T_{ds}T_{ds}^{m}C_{ds}[_{α-L}Tₘₛ]^{m}C_{ds}A_{ds}^{m}C_{ds}A_{ds}TₖₛTₖₛAₖ | 101 | 5 |

In Table 1 above, a subscript “s” indicates a phosphorothioate internucleoside linkage, a subscript “k” represents a cEt modified sugar moiety, a subscript “d” represents a stereo-standard DNA nucleoside, and a superscript “m” indicates 5-methyl Cytosine. A subscript “m2” indicates a substituted stereo-standard nucleoside having a 2′-methylthio modification, which is shown below and wherein Bx is a nucleobase:

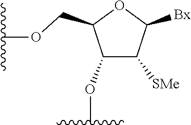

[_{α-L}Bₘₛ] indicates a 2′-substituted stereo-non-standard nucleoside having the alpha-L-ribose configuration and a 2′-OCH₃ modification, which is shown below and wherein Bx is a nucleobase:

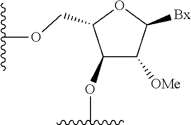

A “[_{α-L}Bₘₛ]^{”} nucleoside is a nucleoside of Formula V, wherein J₉ is H and J₁₀ is OCH₃.

The compounds in Table 1 above are 100% complementary to mouse CXCL12, GENBANK NT_039353.7 truncated from 69430515 to 69445350 (SEQ ID NO: 1), at position 6877 to 6892.

Cultured mouse 3T3-L1 cells at a density of 20,000 cells per well were transfected using electroporation with modified oligonucleotides diluted to 20 μM, 7 μM, 2 μM, 0.7 μM, 0.3 μM, 0.1 μM, and 0.03 μM. After a treatment period of approximately 16 hours, CXCL12 RNA levels were measured using mouse primer-probe set RTS2605 (forward sequence CCAGAGCCAACGTCAAGCAT, SEQ ID NO: 2; reverse sequence: CAGCCGTGCAACAATCTGAA, SEQ ID NO: 3; probe sequence: TGAAAATCCTCAACACTCCAAACTGTGCC, SEQ ID NO: 4). CXCL12 RNA levels were normalized to total RNA content, as measured by RIBOGREEN®. Activity of modified oligonucleotides was calculated using the log (inhibitor) vs response (three parameter) function in GraphPad Prism 7 and is presented in Table 1 above as the half maximal inhibitory concentration (IC₅₀).

### Example 2: Caspase Activity of Modified Oligonucleotides Containing 2′-Substituted Stereo-Standard Nucleosides and 2′-Substituted Stereo-Non-Standard Nucleosides In Vitro

Caspase activity mediated by the modified oligonucleotides was tested in a series of experiments that had similar culture conditions. The results for each experiment are presented in separate tables shown below. Cultured mouse HEPA1-6 cells at a density of 20,000 cells per well were transfected using electroporation with modified oligonucleotides diluted to 20 μM. After a treatment period of approximately 16 hours, caspase-3 and caspase-7 activation was measured using the Caspase-Glo® 3/7 Assay System (G8090, Promega). Increased levels of caspase activation correlate with apoptotic cell death. As seen in the table below, there is a significant reduction in caspase activation and cytotoxicity of the newly designed modified oligonucleotides containing 2′-substituted stereo-standard nucleosides and 2′-substituted stereo-non-standard nucleosides compared to compound 558807.

TABLE 2
In vitro Caspase activation by modified oligonucleotides containing
2′-substituted stereo-standard nucleosides and 2′-substituted
stereo-non-standard nucleosides

| | Caspase |
|---|---|
| Compound | Activation |
| No. | (% Mock) |
| 558807 | 1153 |
| 1385844 | 174 |
| 1385840 | 118 |
| 1385841 | 171 |
| 1385845 | 331 |
| 1385842 | 120 |
| 1385838 | 124 |
| 1385839 | 109 |
| 1385843 | 223 |

### Example 3: Stability of Modified Oligonucleotides Containing 2′-Substituted Stereo-Standard Nucleosides and 2′-Substituted Stereo-Non-Standard Nucleosides

The thermal stability (Tm) of duplexes of each of modified oligonucleotides described in the examples above with a complementary RNA 20-mer having the sequence GAUAAUGUGAGAACAUGCCU (SEQ ID NO: 6) was tested. Each modified oligonucleotide was separately hybridized with the complementary RNA strand to form a duplex. Once the duplex was formed, it was slowly heated and the melting temperature was measured using a spectrophotometer and the hyperchromicity method. Results are provided in Table 3, below. This example demonstrates that 2′-substituted stereo-standard nucleosides and 2′-substituted stereo-non-standard nucleosides can be incorporated into modified oligonucleotides without significantly destabilizing the interaction between the modified oligonucleotide and its complement.

TABLE 3
Tm of modified oligonucleotides complementary to CXCL12

| Compound | Tm |
|---|---|
| No. | (° C.) |
| 558807 | 64.22 |
| 1385844 | 64.37 |
| 1385840 | 61.39 |
| 1385841 | 62.32 |
| 1385845 | 61.27 |
| 1385842 | 60.55 |
| 1385838 | 62.17 |
| 1385839 | 64.49 |
| 1385843 | 64.46 |

### Example 4: In Vivo Activity and Tolerability of Modified Oligonucleotides Containing 2′-Substituted Stereo-Standard Nucleosides and 2′-Substituted Stereo-Non-Standard Nucleosides

Groups of 3 Balb/c mice were injected subcutaneously with 1.9, 5.6, 16.7, 50 and 150 mg/kg of compound 1385838, 1385839, 1385840, or 1385841. One group of three Balb/c mice was injected subcutaneously with 1.8, 5.5, 16.7 and 50 mg/kg of compound 558807. One group of four Balb/c mice was injected with PBS. Mice were euthanized 72 hours following the administration of compound and plasma chemistries and RNA was analyzed.

### Plasma Chemistry Markers

In vivo tolerability of the modified oligonucleotides was determined by measuring plasma levels of aspartate aminotransferase (AST) and alanine aminotransferase (ALT) using an automated clinical chemistry analyzer. All the newly designed modified oligonucleotides show improvement in tolerability markers compared to compound 558807.

TABLE 4
Plasma chemistry markers in vivo

| Compound | Concentration | AST | ALT |
|---|---|---|---|
| No. | (mg/kg) | (IU/L) | (IU/L) |
| PBS | N/A | 30 | 54 |
| 558807 | 50 | 4767 | 6391 |
| | 16.7 | 228 | 270 |
| | 5.5 | 30 | 68 |
| | 1.8 | 41 | 60 |
| | 150 | 41 | 70 |
| 1385838 | 50 | 50 | 104 |
| | 16.7 | 27 | 50 |
| | 5.5 | 34 | 104 |
| | 1.8 | 39 | 76 |
| | 150 | 53 | 116 |
| 1385839 | 50 | 32 | 64 |
| | 16.7 | 39 | 105 |
| | 5.5 | 33 | 50 |
| | 1.8 | 28 | 49 |
| | 150 | 47 | 74 |
| 1385840 | 50 | 47 | 81 |
| | 16.7 | 55 | 86 |
| | 5.5 | 31 | 46 |
| 1385841 | 1.8 | 48 | 86 |
| | 150 | 64 | 115 |
| | 50 | 31 | 49 |
| | 16.7 | 36 | 57 |
| | 5.5 | 37 | 81 |
| | 1.8 | 31 | 55 |

### RNA Analysis

To evaluate the effect of the modified oligonucleotides on CXCL12 levels, CXCL12 RNA levels in liver were measured using mouse primer-probe set RTS2605, which is described in Example 1. CXCL12 RNA levels were normalized to total RNA content, as measured by RIBOGREEN®. Reduction of CXCL12 RNA is presented in the tables below as percent CXCL12 RNA levels relative to saline control.

TABLE 5
Activity of modified oligonucleotides in vivo

| Concentration | CXCL12 mRNA (% PBS control) | | | | |
|---|---|---|---|---|---|
| (mg/kg) | 558807 | 1385838 | 1385839 | 1385840 | 1385841 |
| 150 | N/A | 3 | 15 | 6 | 9 |
| 50 | 2 | 6 | 18 | 10 | 12 |
| 16.7 | 4 | 14 | 29 | 24 | 27 |
| 5.6 | 12 | 35 | 55 | 46 | 50 |
| 1.9 | 46 | 68 | 82 | 64 | 83 |

### Example 5: Effect of Stereo-Non-Standard DNA Nucleosides on In Vitro Activity of Modified Oligonucleotides Complementary to Mouse CXCL12

The newly designed modified oligonucleotides described in Table 6 below have either a 2′-β-D-Xylo-deoxyribosyl stereo-non-standard DNA nucleoside in the gap (a nucleoside of Formula II, wherein J₃ and J₄ are each H), a 2′-α-L-deoxyribosyl stereo-non-standard DNA nucleoside in the gap (a nucleoside of Formula V, wherein J₉ and J₁₀ are each H), or a 2′-substituted stereo-standard modified nucleoside with a 2′-OCH₃ modification in the gap. The precise chemical notation of compound 558807 as well as the newly designed modified oligonucleotides are listed in the table below. A subscript “s” indicates a phosphorothioate internucleoside linkage, a subscript “m” represents a 2′-substituted stereo-standard modified nucleoside with a 2′OCH₃ modification, a subscript “k” represents a cEt modified sugar moiety, a subscript “d” represents a stereo-standard DNA nucleoside, and a superscript “m” indicates 5-methyl Cytosine. [_{β-D-}Bₓₛ] represents a 2′-β-D-Xylo-deoxyribosyl moiety (“β-D-XNA”), which is shown below, wherein Bx is a nucleobase:

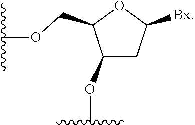

[_{α-L-}B_{ds}] represents a 2′-α-L-deoxyribosyl sugar moiety, which is shown below, wherein Bx is a nucleobase:

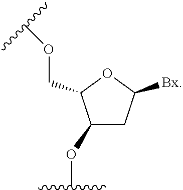

The compounds in Table 6 below are 100% complementary to mouse CXCL12, GENBANK NT_039353.7 truncated from 69430515 to 69445350 (SEQ ID NO: 1), at position 6877 to 6892. The modified oligonucleotides were tested in a series of experiments. The results for each experiment are presented in separate tables shown below. Cultured mouse 3T3-L1 cells at a density of 20,000 cells per well were transfected using electroporation with the modified oligonucleotides diluted to 20 μM, 7 μM, 2 μM, 0.7 μM, 0.3 μM, 0.1 μM, and 0.03 μM. After a treatment period of approximately 16 hours, CXCL12 RNA levels were measured using mouse primer-probe set RTS2605 (forward sequence CCAGAGCCAACGTCAAGCAT, SEQ ID NO: 2; reverse sequence: CAGCCGTGCAACAATCTGAA, SEQ ID NO: 3; probe sequence: TGAAAATCCTCAACACTCCAAACTGTGCC, SEQ ID NO: 4). CXCL12 RNA levels were normalized to total RNA content, as measured by RIBOGREEN®. Activity of the modified oligonucleotides is presented below using the half maximal inhibitory concentration (IC₅₀) values, calculated using the log (inhibitor) vs response (three parameter) function in GraphPad Prism 7. This example demonstrates that modified oligonucleotides having stereo-non-standard DNA nucleosides at certain positions in the gap have similar potency compared to an otherwise identical modified oligonucleotide without any stereo-non-standard DNA nucleosides in the gap.

TABLE 6
Design and activity of modified oligonucleotides having stereo-non-standard DNA
nucleosides

| | position | | | | |
|---|---|---|---|---|---|
| | of altered | sugar | | | |
| | nucleotide | modification | | | SEQ |
| Compound | in central | of altered | | IC₅₀ | ID |
| Number | region | nucleotide | Chemistry Notation (5′-3′) | (nM) | NO |
| 558807 | n/a | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 62 | 5 |
| 1382781 | 1 | β-D-XNA | Gₖₛ^{m}CₖₛAₖₛ[_{β-D}Tₓₛ]G_{ds}T_{ds}T_{ds}^{m}C_{ds}T_{ds} | 77 | 5 |
| | | | ^{m}C_{ds}A_{ds}^{m}C_{ds}A_{ds}TₖₛTₖₛAₖ | | |
| 1382782 | 2 | β-D-XNA | Gₖₛ^{m}CₖₛAₖₛT_{ds}[_{β-D}Gₓₛ] | 120 | 5 |
| | | | T_{ds}T_{ds}^{m}C_{ds}T_{ds}^{m}C_{ds}A_{ds}^{m}C_{ds}A_{ds}TₖₛTₖₛAₖ | | |
| 1263776 | 3 | β-D-XNA | Gₖₛ^{m}CₖₛAₖₛT_{ds}G_{ds}[_{β-D}Tₓₛ] | 110 | 5 |
| | | | T_{ds}^{m}C_{ds}T_{ds}^{m}C_{ds}A_{ds}^{m}C_{ds}A_{ds}TₖₛTₖₛAₖ | | |
| 1263777 | 4 | β-D-XNA | Gₖₛ^{m}CₖₛAₖₛT_{ds}G_{ds}T_{ds}[_{β-D}Tₓₛ] | 66 | 5 |
| | | | ^{m}C_{ds}T_{ds}^{m}C_{ds}A_{ds}^{m}C_{ds}A_{ds}TₖₛTₖₛAₖ | | |
| 1382783 | 5 | β-D-XNA | Gₖₛ^{m}CₖₛAₖₛT_{ds}G_{ds}T_{ds}T_{ds}[_{β-D}^{m}Cₓₛ] | 46 | 5 |
| | | | T_{ds}^{m}C_{ds}A_{ds}^{m}C_{ds}A_{ds}TₖₛTₖₛAₖ | | |
| 1382784 | 6 | β-D-XNA | Gₖₛ^{m}CₖₛAₖₛT_{ds}G_{ds}T_{ds}T_{ds}^{m}C_{ds}[_{β-D}Tₓₛ] | 66 | 5 |
| | | | ^{m}C_{ds}A_{ds}^{m}C_{ds}A_{ds}TₖₛTₖₛAₖ | | |
| 1382785 | 7 | β-D-XNA | Gₖₛ^{m}CₖₛAₖₛT_{ds}G_{ds}T_{ds}T_{ds}^{m}C_{ds}T_{ds}[_{β-} | 52 | 5 |
| | | | _{D}^{m}Cₓₛ]A_{ds}^{m}C_{ds}A_{ds}TₖₛTₖₛAₖ | | |
| 1382786 | 8 | β-D-XNA | Gₖₛ^{m}CₖₛAₖₛT_{ds}G_{ds}T_{ds}T_{ds}^{m}C_{ds}T_{ds}^{m}C_{ds}[_{β-D} | 54 | 5 |
| | | | Aₓₛ]^{m}C_{ds}A_{ds}TₖₛTₖₛAₖ | | |
| 1382787 | 9 | β-D-XNA | Gₖₛ^{m}CₖₛAₖₛT_{ds}G_{ds}T_{ds}T_{ds}^{m}C_{ds}T_{ds}^{m}C_{ds}A_{ds}[_{β-D}^{m} | 44 | 5 |
| | | | Cₓₛ]A_{ds}TₖₛTₖₛAₖ | | |
| 1382788 | 10 | β-D-XNA | Gₖₛ^{m}CₖₛAₖₛT_{ds}G_{ds}T_{ds}T_{ds}^{m}C_{ds}T_{ds}^{m}C_{ds}A_{ds}^{m}C_{ds}[_{β-D} | 66 | 5 |
| | | | Aₓₛ]TₖₛTₖₛAₖ | | |
| 936053 | 2 | 2′-OMe | Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | ND | 5 |
| 1368053 | 2 | α-L DNA | Gₖₛ^{m}CₖₛAₖₛT_{ds}[_{α-L-}G_{ds}] | ND | 5 |
| | | | T_{ds}T_{ds}^{m}C_{ds}T_{ds}^{m}C_{ds}A_{ds}^{m}C_{ds}A_{ds}TₖₛTₖₛAₖ | | |

### Example 6: Caspase Activity of Modified Oligonucleotides Having Stereo-Non-Standard DNA Nucleosides In Vitro

Caspase activity of modified oligonucleotides having stereo-non-standard DNA nucleosides was tested in a series of experiments that had similar culture conditions. The results are presented in Table 7 below. Cultured mouse HEPA1-6 cells at a density of 20,000 cells per well were transfected using electroporation with modified oligonucleotides diluted to 20 μM. After a treatment period of approximately 16 hours, caspase-3 and caspase-7 activation was measured using the Caspase-Glo® 3/7 Assay System (G8090, Promega). Levels of caspase activation correlate with apoptotic cell death. This example demonstrates that placement of stereo-non-standard DNA nucleosides at certain positions in the gap of a modified oligonucleotide reduces cytotoxicity compared to an otherwise identical modified oligonucleotide without any stereo-non-standard DNA nucleosides in the gap.

TABLE 7
In vitro Caspase activation by modified oligonucleotides having
stereo-non-standard DNA nucleosides

| Compound | |
|---|---|
| Number | Caspase % mock |
| 558807 | 1402 |
| 1382781 | 203 |
| 1382782 | 140 |
| 1263776 | 543 |
| 1263777 | 1146 |
| 1382783 | 492 |
| 1382784 | 646 |
| 1382785 | 949 |
| 1382786 | 965 |
| 1382787 | 1352 |
| 1382788 | 1043 |

### Example 7: Stability of Modified Oligonucleotides Having Stereo-Non-Standard DNA Nucleosides

The thermal stability (Tm) of duplexes of each of modified oligonucleotides described in the examples above with a complementary RNA 20-mer having the sequence GAUAAUGUGAGAACAUGCCU (SEQ ID NO: 6) was tested. Each modified oligonucleotide was separately hybridized with the complementary RNA strand to form a duplex. Once the duplex was formed, it was slowly heated and the melting temperature was measured using a spectrophotometer and the hyperchromicity method. Results are provided in Table 8, below. This example demonstrates that stereo-non-standard DNA nucleosides can be incorporated into modified oligonucleotides without destabilizing the interaction between the modified oligonucleotide and its complement.

TABLE 8
Tm of modified oligonucleotides complementary to CXCL12
and having non-standard DNA nucleosides

| Compound | Tm |
|---|---|
| Number | (° C.) |
| 558807 | 64.4 |
| 1382781 | 63.8 |
| 1382782 | 63.4 |
| 1263776 | 63.1 |
| 1263777 | 64.8 |
| 1382783 | 63.1 |
| 1382784 | 63.2 |
| 1382785 | 64.8 |
| 1382786 | 63.2 |
| 1382787 | 65.2 |
| 1382788 | 63.2 |

### Example 8: In Vivo Activity and Tolerability of Modified Oligonucleotides Having Stereo-Non-Standard DNA Nucleosides

Groups of 3 Balb/c mice were injected subcutaneously with 1.8, 5.5, 16.7, 50 and 150 mg/kg of compound 1368053, 1382781, 1382782, or 936053. One group of four Balb/c mice was injected with PBS. Mice were euthanized 72 hours following the subcutaneous injection, and plasma chemistry and RNA was analyzed.

### Plasma Chemistry Markers

In vivo tolerability of the modified oligonucleotides was determined by measuring plasma levels of aspartate aminotransferase (AST) and alanine aminotransferase (ALT) using an automated clinical chemistry analyzer. The newly designed modified oligonucleotides having stereo-non-standard DNA nucleosides show good tolerability over a range of doses, including comparable tolerability to a modified oligonucleotide having a 2′-substituted stereo-standard nucleoside with a 2′-OCH₃ modification at the 2 position of the gap (compound 936053). For mice injected with PBS, ALT is observed to be 28 IU/L, and AST is 37 IU/L.

TABLE 9
Plasma chemistry markers in vivo

| | position | | | | | | |
|---|---|---|---|---|---|---|---|
| | of altered | sugar | | | | | |
| | nucleotide | modification | ALT (IU/L) | | | | |
| Compound | in central | of altered | 150 | 50 | 16.7 | 5.5 | 1.8 |
| Number | region | nucleotide | mg/kg | mg/kg | mg/kg | mg/kg | mg/kg |
| 936053 | 2 | 2′-OMe | 56 | 36 | 31 | 26 | 33 |
| 1368053 | 2 | α-L DNA | 125 | 35 | 33 | 23 | 33 |
| 1382781 | 1 | β-D-XNA | 2389 | 92 | 28 | 24 | 31 |
| 1382782 | 2 | β-D-XNA | 34 | 28 | 36 | 32 | 35 |

TABLE 10
Plasma chemistry markers in vivo

| | position | | | | | | |
|---|---|---|---|---|---|---|---|
| | of altered | sugar | | | | | |
| | nucleotide | modification | AST (IU/L) | | | | |
| Compound | in central | of altered | 150 | 50 | 16.7 | 5.5 | 1.8 |
| Number | region | nucleotide | mg/kg | mg/kg | mg/kg | mg/kg | mg/kg |
| 936053 | 2 | 2′-OMe | 61 | 46 | 46 | 40 | 43 |
| 1368053 | 2 | α-L DNA | 109 | 58 | 44 | 48 | 43 |
| 1382781 | 1 | β-D-XNA | 1692 | 124 | 44 | 38 | 47 |
| 1382782 | 2 | β-D-XNA | 44 | 40 | 46 | 61 | 51 |

### RNA Analysis

To evaluate the effect of the modified oligonucleotides on CXCL12 levels, CXCL12 RNA levels in liver were measured using mouse primer-probe set RTS2605, which is described in Example 1. CXCL12 RNA levels were normalized to total RNA content, as measured by RIBOGREEN®. Reduction of CXCL12 RNA is presented in the tables below as percent CXCL12 RNA levels relative to saline control.

TABLE 11
Activity of sugar-modified oligonucleotides in vivo

| Concentration | CXCL12 mRNA (% PBS) | | | |
|---|---|---|---|---|
| (mg/kg) | 936053 | 1368053 | 1382781 | 1382782 |
| 150 | 10 | 3 | 4 | 6 |
| 50 | 13 | 5 | 8 | 9 |
| 16.7 | 19 | 10 | 13 | 12 |
| 5.5 | 38 | 22 | 20 | 22 |
| 1.8 | 55 | 40 | 39 | 45 |

This example demonstrates that modified oligonucleotides having stereo-non-standard DNA nucleosides in the gap have similar tolerability over a range of doses as compared to a modified oligonucleotide having a 2′-substituted stereo-standard nucleoside with a 2′-OCH₃ modification at the 2 position of the gap. Additionally, modified oligonucleotides having stereo-non-standard DNA nucleosides in the gap have better potency as compared to a modified oligonucleotide having a 2′-substituted stereo-standard nucleoside with a 2′-OCH₃ modification at the 2 position of the gap.

### Example 9: In Vivo Activity and Tolerability of Modified Oligonucleotides Having Stereo-Non-Standard DNA Nucleosides

Groups of 3 Balb/c mice were injected subcutaneously with 10 and 150 mg/kg of newly synthesized compounds 1263776, 1263777, or 936053. One group of four Balb/c mice was injected with PBS. Mice were euthanized 72 hours following the administration of compound. Plasma chemistry and RNA was then analyzed.

### Plasma Chemistry Markers

In vivo tolerability of the modified oligonucleotides was determined by measuring plasma levels of aspartate aminotransferase (AST), alanine aminotransferase (ALT) using an automated clinical chemistry analyzer. For mice injected with PBS, ALT is observed to be 26 IU/L, and AST is 53 IU/L.

TABLE 12
Plasma chemistry markers in vivo

| | position | | | |
|---|---|---|---|---|
| | of altered | sugar | | |
| | nucleotide | modification | ALT (IU/L) | |
| Compound | in central | of altered | 150 | 10 |
| Number | region | nucleotide | mg/kg | mg/kg |
| 936053 | 2 | 2′-OMe | 83 | 23 |
| 1263776 | 3 | β-D-XNA | 9234 | 27 |
| 1263777 | 4 | β-D-XNA | ND | 58 |

TABLE 13
Plasma chemistry markers in vivo

| | position | | | |
|---|---|---|---|---|
| | of altered | sugar | | |
| | nucleotide | modification | AST (IU/L) | |
| Compound | in central | of altered | 150 | 10 |
| Number | region | nucleotide | mg/kg | mg/kg |
| 936053 | 2 | 2′-OMe | 88 | 45 |
| 1263776 | 3 | β-D-XNA | 10075 | 54 |
| 1263777 | 4 | β-D-XNA | ND | 102 |

### RNA Analysis

To evaluate the effect of the modified oligonucleotides on CXCL12 levels, CXCL12 RNA levels in liver were measured using mouse primer-probe set RTS2605, which is described in Example 1. CXCL12 RNA levels were normalized to total RNA content, as measured by RIBOGREEN®. Reduction of CXCL12 RNA is presented in the tables below as percent CXCL12 RNA levels relative to saline control.

TABLE 14
Activity of sugar-modified oligonucleotides in vivo

| Concentration | CXCL12 mRNA (% PBS) | | |
|---|---|---|---|
| (mg/kg) | 936053 | 1263776 | 1263777 |
| 150 | 13 | 11 | ND |
| 10 | 37 | 19 | 13 |

### Example 10: In Vivo Activity and Tolerability of Modified Oligonucleotides Having Stereo-Non-Standard DNA Nucleosides

Modified oligonucleotides having a stereo-non-standard DNA nucleoside at positions 1-5 of the gap were synthesized using standard techniques or those described herein and are described in Table 15 below. The compounds in Table 15 below are 100% complementary to mouse CXCL12, GENBANK NT_039353.7 truncated from 69430515 to 69445350 (SEQ ID NO: 1), at position 6877 to 6892.

In Table 15 below, a subscript “s” indicates a phosphorothioate internucleoside linkage, a subscript “k” represents a cEt modified sugar moiety, a subscript “d” represents a stereo-standard DNA nucleoside, and a superscript “m” indicates 5-methyl Cytosine.

[_{α-L-}B_{ds}] represents a 2′-α-L-deoxyribosyl sugar moiety, which is shown below, wherein Bx is a nucleobase:

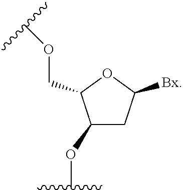

A “[_{α-L-}B_{ds}]” nucleoside is a nucleoside of Formula V, wherein J₉ and J₁₀ are each H.

TABLE 15
Modified oligonucleotides complementary to CXCL12

| | position | | | |
|---|---|---|---|---|
| | of altered | sugar | | |
| | nucleotide | modification | | SEQ |
| Compound | in central | of altered | | ID |
| Number | region | nucleotide | Chemistry Notation (5′-3′) | NO |
| 558807 | n/a | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 5 |
| 1368034 | 1 | α-L DNA | Gₖₛ^{m}CₖₛAₖₛ[_{α-L}T_{ds}] | 5 |
| | | | G_{ds}T_{ds}T_{ds}^{m}C_{ds}T_{ds}^{m}C_{ds}A_{ds}^{m}C_{ds}A_{ds}TₖₛTₖₛAₖ | |
| 1368053 | 2 | α-L DNA | Gₖₛ^{m}CₖₛAₖₛT_{ds}[_{α-L}G_{ds}] | 5 |
| | | | T_{ds}T_{ds}^{m}C_{ds}T_{ds}^{m}C_{ds}A_{ds}^{m}C_{ds}A_{ds}TₖₛTₖₛAₖ | |
| 1215461 | 3 | α-L DNA | Gₖₛ^{m}CₖₛAₖₛT_{ds}G_{ds}[_{α-L}T_{ds}] | 5 |
| | | | T_{ds}^{m}C_{ds}T_{ds}^{m}C_{ds}A_{ds}^{m}C_{ds}A_{ds}TₖₛTₖₛAₖ | |
| 1215462 | 4 | α-L DNA | Gₖₛ^{m}CₖₛAₖₛT_{ds}G_{ds}T_{ds}[_{α-L}T_{ds}] | 5 |
| | | | ^{m}C_{ds}T_{ds}^{m}C_{ds}A_{ds}^{m}C_{ds}A_{ds}TₖₛTₖₛAₖ | |
| 1368054 | 5 | α-L DNA | Gₖₛ^{m}CₖₛAₖₛT_{ds}G_{ds}T_{ds}T_{ds}[_{α-L}^{m}C_{ds}] | 5 |
| | | | T_{ds}^{m}C_{ds}A_{ds}^{m}C_{ds}A_{ds}TₖₛTₖₛAₖ | |

Groups of 3 Balb/c mice were injected subcutaneously with 1.8, 5.5, 16.7, 50 and 150 mg/kg of newly synthesized modified oligonucleotides 1368034, 1368053, 1215461, 1215462, or 1368054. One group of three Balb/c mice was injected subcutaneously with 1.8, 5.5, 16.7 and 50 mg/kg of compound 558807. One group of four Balb/c mice was injected with PBS. Mice were euthanized 72 hours following the administration of compound. Plasma chemistry and RNA was then analyzed.

### Plasma Chemistry Markers

In vivo tolerability of the modified oligonucleotides was determined by measuring plasma levels of aspartate aminotransferase (AST), alanine aminotransferase (ALT) using an automated clinical chemistry analyzer. All the newly designed modified oligonucleotides having a stereo-non-standard DNA nucleoside show improvement in tolerability markers compared to compound 558807. For mice injected with PBS, ALT is observed to be 23 IU/L, and AST is 43 IU/L.

TABLE 16
Plasma chemistry markers in vivo

| | position of | | | | | | |
|---|---|---|---|---|---|---|---|
| | altered | sugar | | | | | |
| | nucleotide | modification | ALT (IU/L) | | | | |
| Compound | in central | of altered | 150 | 50 | 16.7 | 5.5 | 1.8 |
| Number | region | nucleotide | mg/kg | mg/kg | mg/kg | mg/kg | mg/kg |
| 558807 | n/a | n/a | n/a | 4035 | 273 | 26 | 40 |
| 1368034 | 1 | α-L DNA | 47 | 50 | 30 | 29 | 27 |
| 1368053 | 2 | α-L DNA | 50 | 39 | 23 | 23 | 29 |
| 1215461 | 3 | α-L DNA | 4561 | 667 | 28 | 27 | 27 |
| 1215462 | 4 | α-L DNA | 933 | 45 | 22 | 35 | 26 |
| 1368054 | 5 | α-L DNA | 1190 | 100 | 30 | 40 | 28 |

TABLE 17
Plasma chemistry markers in vivo

| | position of | | | | | | |
|---|---|---|---|---|---|---|---|
| | altered | sugar | | | | | |
| | nucleotide | modification | AST (IU/L) | | | | |
| Compound | in central | of altered | 150 | 50 | 16.7 | 5.5 | 1.8 |
| Number | region | nucleotide | mg/kg | mg/kg | mg/kg | mg/kg | mg/kg |
| 558807 | n/a | n/a | n/a | 4870 | 328 | 69 | 80 |
| 1368034 | 1 | α-L DNA | 73 | 86 | 53 | 63 | 57 |
| 1368053 | 2 | α-L DNA | 74 | 122 | 68 | 48 | 111 |
| 1215461 | 3 | α-L DNA | 4815 | 636 | 58 | 53 | 47 |
| 1215462 | 4 | α-L DNA | 1135 | 107 | 47 | 64 | 47 |
| 1368054 | 5 | α-L DNA | 914 | 104 | 49 | 72 | 89 |

RNA analysis

To evaluate the effect of the modified oligonucleotides on CXCL12 levels, CXCL12 RNA levels in liver were measured using mouse primer-probe set RTS2605, which is described in Example 1. CXCL12 RNA levels were normalized to total RNA content, as measured by RIBOGREEN®. Reduction of CXCL12 RNA is presented in the tables below as percent CXCL12 RNA levels relative to saline control.

TABLE 18
Activity of sugar-modified oligonucleotides in vivo

| Concentration | CXCL12 mRNA (% PBS) | | | | | |
|---|---|---|---|---|---|---|
| (mg/kg) | 558807 | 1368034 | 1368053 | 1215461 | 1215462 | 1368054 |
| 150 | n/a | 9 | 5 | 3 | 4 | 2 |
| 50 | 4 | 10 | 8 | 3 | 5 | 4 |
| 16.7 | 4 | 18 | 12 | 5 | 11 | 7 |
| 5.5 | 12 | 35 | 31 | 20 | 26 | 21 |
| 1.8 | 43 | 73 | 62 | 51 | 53 | 53 |

### Example 11: Stereochemical Isomers of Nucleosides

Modified oligonucleotides containing modified nucleotides with various stereochemical configurations at positions 1′, 3′, and 5′ of the 2′-deoxyfuranosyl sugar were synthesized using standard techniques or those described herein. Amidites for the synthesis of the stereo-non-standard β-L-DNA-containing nucleotides are commercially available (ChemGenes) and the synthesis of both α-L and β-L dT phosphoramidites has been reported (Morvan, Biochem and Biophys Research Comm, 172(2): 537-543, 1990). The altered stereo-non-standard DNA nucleotides were contained within the central region of the oligonucleotide.

These modified oligonucleotides were compared to the otherwise identical modified oligonucleotide lacking a an altered nucleotide in the central region, 558807, described in Table 1, Example 1 above. The compounds in Table 19 each comprise a 5′ wing and a 3′ wing each consisting of three linked cEt nucleosides and a central region comprising nucleosides each comprising 2′-β-D-deoxyribosyl sugar moieties aside from the altered nucleotide, as indicated. Each internucleoside linkage is a phosphodiester internucleoside linkage. The compounds in the table below are 100% complementary to mouse CXCL12, GENBANK NT_039353.7 truncated from 69430515 to 69445350 (SEQ ID NO: 1), at position 6877 to 6892.

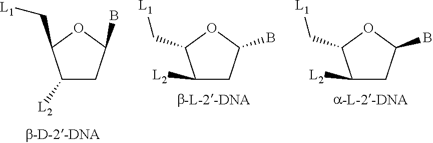

B is any nucleobase and L₁ and L₂ are internucleoside linkages
   A β-L-2′DNA is a nucleoside of Formula IV, wherein J₇ and J₈ are each H. An α-L DNA is a nucleoside of Formula V, wherein J₉ and J₁₀ are each H.

TABLE 19
modified oligonucleotides with stereochemical modifications

| | position | stereo- | | |
|---|---|---|---|---|
| | of altered | chemical | | |
| | nucleotide | configuration | | SEQ |
| Compound | in central | of altered | | ID |
| Number | region | nucleotide | Chemistry Notation (5′-3′) | NO |
| 1215458 | 2 | β-L-DNA | Gₖₛ^{m}CₖₛAₖₛT_{ds}[_{β-} | 5 |
| | | | _{L}G_{ds}]T_{ds}T_{ds}^{m}C_{ds}T_{ds}^{m}C_{ds}A_{ds}^{m}C_{ds}A_{ds}TₖₛTₖₛAₖ | |
| 1215459 | 3 | β-L-DNA | Gₖₛ^{m}CₖₛAₖₛT_{ds}G_{ds}[_{β-} | 5 |
| | | | _{L}T_{ds}]T_{ds}^{m}C_{ds}T_{ds}^{m}C_{ds}A_{ds}^{m}C_{ds}A_{ds}TₖₛTₖₛAₖ | |
| 1215460 | 4 | β-L-DNA | Gₖₛ^{m}CₖₛAₖₛT_{ds}G_{ds}T_{ds}[_{β-L} | 5 |
| | | | T_{ds}]^{m}C_{ds}T_{ds}^{m}C_{ds}A_{ds}^{m}C_{ds}A_{ds}TₖₛTₖₛAₖ | |
| 1215461 | 3 | α-L-DNA | Gₖₛ^{m}CₖₛAₖₛT_{ds}G_{ds}[_{α-} | 5 |
| | | | _{L}T_{ds}]T_{ds}^{m}C_{ds}T_{ds}^{m}C_{ds}A_{ds}^{m}C_{ds}A_{ds}TₖₛTₖₛAₖ | |
| 1215462 | 4 | α-L-DNA | Gₖₛ^{m}CₖₛAₖₛT_{ds}G_{ds}T_{ds}[_{α-} | 5 |
| | | | _{L}T_{ds}]^{m}C_{ds}T_{ds}^{m}C_{ds}A_{ds}^{m}C_{ds}A_{ds}TₖₛTₖₛAₖ | |

A subscript “d” indicates an unmodified, 2′-β-D-deoxyribosyl sugar moiety. A subscript “k” indicates a cEt. A subscript “s” indicates a phosphorothioate mtemucleoside linkage. [_{β-L}B_{ds}] indicates a modified β-L-DNA nucleotide with a 2′-deoxyribosyl moiety, a phosphorothioate linkage, and base B. [_{α-L}B_{ds}] indicates a modified, α-L DNA nucleotide with a 2′-deoxyribosyl sugar moiety, a phosphorothioate linkage, and base B.

For in vitro activity and toxicity studies, approximately 20,000 mouse 3T3-L1 cells were electroporated with 0, 27 nM, 80 nM, 250 nM, 740 nM, 2, 222 nM, 6,667 nM, or 20,000 nM of modified oligonucleotide. mRNA was harvested and analyzed by RT-qPCR. CXCL12 mRNA was detected with primer probe set RTS2605 (forward sequence CCAGAGCCAACGTCAAGCAT, SEQ ID NO: 2; reverse sequence: CAGCCGTGCAACAATCTGAA, SEQ ID NO: 3; probe sequence: TGAAAATCCTCAACACTCCAAACTGTGCC, SEQ ID NO: 4) and RAPTOR mRNA was detected with primer probe set RTS3420 (forward sequence GCCCTCAGAAAGCTCTGGAA, SEQ ID NO: 7; reverse sequence: TAGGGTCGAGGCTCTGCTTGT, SEQ ID NO: 8; probe sequence: CCATCGGTGCAAACCTACAGAAGCAGTATG, SEQ ID NO: 9). RAPTOR is a sentinel gene that can be indicative of toxicity, as described in US 20160160280, hereby incorporated by reference.

For the in vitro study reported in the tables below, 3T3-L1 cells were electroporated with 27 nM, 80 nM, 250 nM, 740 nM, 2, 222 nM, 6,667 nM, or 20,000 nM of modified oligonucleotide and levels of P21, Gadd45a and Tnfrsf10b were measured by RT-qPCR. Levels of Gadd45a were analyzed using primer probe set Mm00432802_m1 (ThermoFisher). Levels of P21 were analyzed using primer probe set Mm04207341_m1 (ThermoFisher). Levels of Tnfrsf10b were analyzed using primer probe set Mm004578866_m1 (ThermoFisher). Expression levels were normalized with Ribogreen® and are presented relative to levels in mice treated with PBS.

Caspase-3 and caspase-7 activation was measured using the Caspase-Glo® 3/7 Assay System (G8090, Promega). Levels of caspase activation correlate with apoptotic cell death. Results are presented relative to the caspase activation in control cells not treated with modified oligonucleotide.

For the in vivo activity study in the tables below, 2 BALB/C mice per group were administered 1.8 mg/kg, 5.5 mg/kg, 16.7 mg/kg, 50 mg/kg, or 150 mg/kg doses of modified oligonucleotide, as indicated in the table below, by subcutaneous injection and sacrificed 72 hours later. For 558807, only 1.8 mg/kg, 5.5 mg/kg, and 16.7 mg/kg doses were tested for dose response, due to acute toxicity of higher doses. Plasma levels of ALT were measured using an automated clinical chemistry analyzer. Increased ALT is indicative of acute liver toxicity. Liver mRNA was isolated and analyzed by RT-PCR as described above. Expression levels were normalized with Ribogreen® and are expressed relative to PBS-treated control mice.

TABLE 20
Activity and toxicity of modified oligonucleotides complementary
CXCL12

| | in vitro | | in vivo | | |
|---|---|---|---|---|---|
| | CXCL12 | in vitro | CXCL12 | ALT @ | ALT @ |
| Compound | IC₅₀ | RAPTOR | ED₅₀ | 50 mg/kg | 150 mg/kg |
| ID | (μM) | IC₅₀ (μM) | (mg/kg) | (IU/L) | (IU/L) |
| PBS | n/a | n/a | n/a | 25 @ 0 mg/kg | |
| 558807 | 0.10 | 1 | 2.9 | n.d.** | |
| 1215458 | 0.41 | >20 | 11 | 32 | 42 |
| 1215459 | 0.44 | >20 | 13 | 31 | 37 |
| 1215460 | 0.41 | >20 | 13 | 29 | 43 |
| 1215461 | 0.14 | 3 | 2.8 | 1725 | 6301 |
| 1215462 | 0.13 | 3 | 3.6 | 45 | 3652 |

**558807 treatment at 16.7 mg/kg leads to an ALT of 586 IU/L; mice that are treated with 558807 at 150 mg/kg typically experience death before 72 hours post-treatment.

TABLE 21
in vitro Caspase Activation

| | 27 | 80 | 250 | 740 | 2,222 | 6,667 | 20,000 |
|---|---|---|---|---|---|---|---|
| | nM | nM | nM | nM | nM | nM | nM |
| Compound ID | Relative Caspase Activation (% Control) | | | | | | |
| 558807 | 106 | 113 | 117 | 169 | 250 | 396 | 343 |
| 1215458 | 81 | 88 | 98 | 95 | 74 | 78 | 95 |
| 1215459 | 96 | 88 | 111 | 98 | 98 | 81 | 102 |
| 1215460 | 89 | 98 | 96 | 111 | 91 | 113 | 130 |
| 1215461 | 90 | 94 | 89 | 117 | 142 | 201 | 250 |
| 1215462 | 96 | 93 | 95 | 119 | 150 | 192 | 240 |

TABLE 21b
in vitro P21 Expression

| | 27 | 80 | 250 | 740 | 2,222 | 6,667 | 20,000 |
|---|---|---|---|---|---|---|---|
| | nM | nM | nM | nM | nM | nM | nM |
| Compound ID | Expression level of P21 mRNA (% Control) | | | | | | |
| 558807 | 98 | 116 | 122 | 115 | 115 | 135 | 184 |
| 1215458 | 104 | 127 | 135 | 153 | 139 | 140 | 130 |
| 1215459 | 99 | 116 | 134 | 154 | 158 | 141 | 147 |
| 1215460 | 85 | 109 | 118 | 120 | 118 | 122 | 109 |
| 1215461 | 105 | 107 | 128 | 136 | 139 | 147 | 153 |
| 1215462 | 110 | 127 | 143 | 150 | 139 | 124 | 143 |

TABLE 21c
in vitro Tnfrsf10b Expression

| | 27 | 80 | 250 | 740 | 2,222 | 6,667 | 20,000 |
|---|---|---|---|---|---|---|---|
| | nM | nM | nM | nM | nM | nM | nM |
| Compound ID | Expression level of Tnfrsf10b mRNA (% Control) | | | | | | |
| 558807 | 107 | 108 | 105 | 99 | 113 | 102 | 68 |
| 1215458 | 90 | 88 | 92 | 87 | 81 | 78 | 80 |
| 1215459 | 97 | 108 | 108 | 100 | 103 | 94 | 83 |
| 1215460 | 92 | 100 | 99 | 102 | 95 | 95 | 84 |
| 1215461 | 86 | 91 | 99 | 98 | 97 | 97 | 114 |
| 1215462 | 101 | 97 | 98 | 56 | 82 | 101 | 108 |

TABLE 21d
in vitro Gadd45a Expression

| | 27 | 80 | 250 | 740 | 2,222 | 6,667 | 20,000 |
|---|---|---|---|---|---|---|---|
| | nM | nM | nM | nM | nM | nM | nM |
| Compound ID | Expression level of Gadd45a mRNA (% Control) | | | | | | |
| 558807 | 123 | 134 | 135 | 136 | 164 | 180 | 223 |
| 1215458 | 132 | 142 | 141 | 135 | 125 | 104 | 87 |
| 1215459 | 163 | 167 | 183 | 190 | 179 | 150 | 110 |
| 1215460 | 127 | 142 | 140 | 141 | 143 | 120 | 95 |
| 1215461 | 117 | 141 | 150 | 165 | 168 | 167 | 128 |
| 1215462 | 110 | 139 | 143 | 138 | 133 | 150 | 137 |

### Example 12: Stereo-Non-Standard Nucleosides

Modified oligonucleotides containing stereo-non-standard β-L-DNA nucleotides (described in Example 11 above) at various positions were synthesized standard techniques or those described herein. These modified oligonucleotides were compared to compound 558807, described in Table 1, Example 1 above. Compound 558807 contains 5-methyl cytosine for all cytosine nucleosides, as do compounds 1215458-1215460 described in the table below. The compounds in Table 22 each comprise a 5′ wing and a 3′ wing each consisting of three linked cEt nucleosides and a central region comprising nucleosides each comprising 2′-β-D-deoxyribosyl sugar moieties aside from the altered nucleotide, as indicated. Each internucleoside linkage is a phosphodiester internucleoside linkage. Compounds 1244441-1244447 in the table below contain unmethylated cytosine in the central region of the compounds. The compounds in the table below are 100% complementary to mouse CXCL12, GENBANK NT_039353.7 truncated from 69430515 to 69445350 (SEQ ID NO: 1), at position 6877 to 6892.

TABLE 22
modified oligonucleotides with stereo-non-standard nucleosides

| | position | stereo- | | |
|---|---|---|---|---|
| | of altered | chemical | | |
| | nucleotide | configuration | | SEQ |
| Compound | in central | of altered | | ID |
| Number | region | nucleotide | Chemistry Notation | NO |
| 1244441 | 1 | β-L-DNA | Gₖₛ^{m}CₖₛAₖₛ[_{β-L}T_{ds}]G_{ds}T_{ds}T_{ds}C_{ds}T_{ds}C_{ds}A_{ds}C_{ds}A_{ds}TₖₛTₖₛAₖ | 5 |
| 1215458 | 2 | β-L-DNA | Gₖₛ^{m}CₖₛAₖₛT_{ds}[_{β-L}G_{ds}] | 5 |
| | | | T_{ds}T_{ds}^{m}C_{ds}T_{ds}^{m}C_{ds}A_{ds}^{m}C_{ds}A_{ds}TₖₛTₖₛAₖ | |
| 1215459 | 3 | β-L-DNA | Gₖₛ^{m}CₖₛAₖₛT_{ds}G_{ds}[_{β-L}T_{ds}] | 5 |
| | | | T_{ds}^{m}C_{ds}T_{ds}^{m}C_{ds}A_{ds}^{m}C_{ds}A_{ds}TₖₛTₖₛAₖ | |
| 1215460 | 4 | β-L-DNA | Gₖₛ^{m}CₖₛAₖₛT_{ds}G_{ds}T_{ds}[_{β-L}T_{ds}] | 5 |
| | | | ^{m}C_{ds}T_{ds}^{m}C_{ds}A_{ds}^{m}C_{ds}A_{ds}TₖₛTₖₛAₖ | |
| 1244442 | 5 | β-L-DNA | Gₖₛ^{m}CₖₛAₖₛT_{ds}G_{ds}T_{ds}T_{ds}[_{β-L}C_{ds}]T_{ds}C_{ds}A_{ds}C_{ds}A_{ds}TₖₛTₖₛAₖ | 5 |
| 1244443 | 6 | β-L-DNA | Gₖₛ^{m}CₖₛAₖₛT_{ds}G_{ds}T_{ds}T_{ds}C_{ds}[_{β-L}T_{ds}]C_{ds}A_{ds}C_{ds}A_{ds}TₖₛTₖₛAₖ | 5 |
| 1244444 | 7 | β-L-DNA | Gₖₛ^{m}CₖₛAₖₛT_{ds}G_{ds}T_{ds}T_{ds}C_{ds}T_{ds}[_{β-L}C_{ds}]A_{ds}C_{ds}A_{ds}TₖₛTₖₛAₖ | 5 |
| 1244445 | 8 | β-L-DNA | Gₖₛ^{m}CₖₛAₖₛT_{ds}G_{ds}T_{ds}T_{ds}C_{ds}T_{ds}C_{ds}[_{β-L}A_{ds}]C_{ds}A_{ds}TₖₛTₖₛAₖ | 5 |
| 1244446 | 9 | β-L-DNA | Gₖₛ^{m}CₖₛAₖₛT_{ds}G_{ds}T_{ds}T_{ds}C_{ds}T_{ds}C_{ds}A_{ds}[_{β-L}C_{ds}]A_{ds}TₖₛTₖₛAₖ | 5 |
| 1244447 | 10 | β-L-DNA | Gₖₛ^{m}CₖₛAₖₛT_{ds}G_{ds}T_{ds}T_{ds}C_{ds}T_{ds}C_{ds}A_{ds}C_{ds}[_{β-L}A_{ds}]TₖₛTₖₛAₖ | 5 |

A subscript “d” indicates a nucleoside comprising an unmodified, 2′-β-D-deoxyribosyl sugar moiety. A subscript “k“ indicates a cEt. A subscript “s” indicates a phosphorothioate internucleoside linkage. [_{β-L}B_{ds}] indicates a modified β-L-DNA nucleotide with a 2′-deoxyribosyl sugar moiety, a phosphorothioate linkage, and base B.

For the results in the tables below, in vitro activity and toxicity experiments were performed essentially as described in Example 11. For in vitro activity and toxicity studies, 3T3-L1 cells were transfected with 27 nM, 80 nM, 250 nM, 740 nM, 2, 222 nM, 6,667 nM, or 20,000 nM of modified oligonucleotide by electroporation and levels of P21, Gadd45a and Tnfrsf10b were measured by RT-qPCR as described in Example 11 above. The caspase assay was performed as described in Example 11 above in 3T3-L1 cells.

TABLE 23
In vitro activity and toxicity of modified oligonucleotides
complementary to CXCL12

| | in vitro | in vitro |
|---|---|---|
| | CXCL12 | Caspase |
| Compound | IC₅₀ | (% control) |
| ID | (nM) | @ 20 μM |
| 558807 | 0.029 | 321 |
| 1244441 | 0.471 | 108 |
| 1215458 | 0.200 | 104 |
| 1215459 | 0.191 | 111 |
| 1215460 | 0.130 | 133 |
| 1244442 | 0.134 | 185 |
| 1244443 | 0.083 | 279 |
| 1244444 | 0.109 | 213 |
| 1244445 | 0.198 | 249 |
| 1244446 | 0.127 | 243 |
| 1244447 | 0.080 | 333 |

### Example 13: Stereochemical Isomers of Nucleosides

Modified oligonucleotides containing stereo-non-standard α-D-DNA nucleotides (see below) at various positions were synthesized using standard techniques or those described herein. These modified oligonucleotides were compared to the otherwise identical modified oligonucleotide lacking an altered nucleotide in the central region. The compounds in Table 24 each comprise a 5′ wing and a 3′ wing each consisting of three linked cEt nucleosides and a central region comprising nucleosides each comprising 2′-β-D-deoxyribosyl sugar moieties aside from the altered nucleotide, as indicated. Each internucleoside linkage is a phosphodiester internucleoside linkage. The compounds in the table below are 100% complementary to mouse CXCL12, GENBANK NT_039353.7 truncated from 69430515 to 69445350 (SEQ ID NO: 1), at position 6877 to 6892.

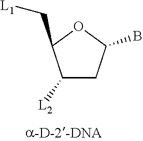

An α-D-DNA is a nucleoside of Formula I, wherein J₁ and J₂ are each H.

TABLE 24
modified oligonucleotides with stereochemical modifications

| | position | stereo- | | |
|---|---|---|---|---|
| | of altered | chemical | | |
| | nucleotide | configuration | | SEQ |
| Compound | in central | of altered | | ID |
| Number | region | nucleotide | Chemistry Notation | NO |
| 1244458 | none | none | Gₖₛ^{m}CₖₛAₖₛT_{ds}G_{ds}T_{ds}T_{ds}C_{ds}T_{ds}C_{ds}A_{ds}C_{ds}A_{ds}TₖₛTₖₛAₖ | 5 |
| 1244448 | 1 | α-D-DNA | Gₖₛ^{m}CₖₛAₖₛ[_{α-D}T_{ds}]G_{ds}T_{ds}T_{ds}C_{ds}T_{ds}C_{ds}A_{ds}C_{ds}A_{ds}TₖₛTₖₛAₖ | 5 |
| 1244449 | 2 | α-D-DNA | Gₖₛ^{m}CₖₛAₖₛT_{ds}[_{α-D}G_{ds}]T_{ds}T_{ds}C_{ds}T_{ds}C_{ds}A_{ds}C_{ds}A_{ds}TₖₛTₖₛAₖ | 5 |
| 1244450 | 3 | α-D-DNA | Gₖₛ^{m}CₖₛAₖₛT_{ds}G_{ds}[_{α-D}T_{ds}]T_{ds}C_{ds}T_{ds}C_{ds}A_{ds}C_{ds}A_{ds}TₖₛTₖₛAₖ | 5 |
| 1244451 | 4 | α-D-DNA | Gₖₛ^{m}CₖₛAₖₛT_{ds}G_{ds}T_{ds}[_{α-D}T_{ds}]C_{ds}T_{ds}C_{ds}A_{ds}C_{ds}A_{ds}TₖₛTₖₛAₖ | 5 |
| 1244452 | 5 | α-D-DNA | Gₖₛ^{m}CₖₛAₖₛT_{ds}G_{ds}T_{ds}T_{ds}[_{α-D}C_{ds}]T_{ds}C_{ds}A_{ds}C_{ds}A_{ds}TₖₛTₖₛAₖ | 5 |
| 1244453 | 6 | α-D-DNA | Gₖₛ^{m}CₖₛAₖₛT_{ds}G_{ds}T_{ds}T_{ds}C_{ds}[_{α-D}T_{ds}]C_{ds}A_{ds}C_{ds}A_{ds}TₖₛTₖₛAₖ | 5 |
| 1244454 | 7 | α-D-DNA | Gₖₛ^{m}CₖₛAₖₛT_{ds}G_{ds}T_{ds}T_{ds}C_{ds}T_{ds}[_{α-D}C_{ds}]A_{ds}C_{ds}A_{ds}TₖₛTₖₛAₖ | 5 |
| 1244455 | 8 | α-D-DNA | Gₖₛ^{m}CₖₛAₖₛT_{ds}G_{ds}T_{ds}T_{ds}C_{ds}T_{ds}C_{ds}[_{α-D}A_{ds}]C_{ds}A_{ds}TₖₛTₖₛAₖ | 5 |
| 1244456 | 9 | α-D-DNA | Gₖₛ^{m}CₖₛAₖₛT_{ds}G_{ds}T_{ds}T_{ds}C_{ds}T_{ds}C_{ds}A_{ds}[_{α-D}C_{ds}]A_{ds}TₖₛTₖₛAₖ | 5 |
| 1244457 | 10 | α-D-DNA | Gₖₛ^{m}CₖₛAₖₛT_{ds}G_{ds}T_{ds}T_{ds}C_{ds}T_{ds}C_{ds}A_{ds}C_{ds}[_{α-D}A_{ds}]TₖₛTₖₛAₖ | 5 |

A subscript “d” indicates a nucleoside comprising an unmodified, 2′-β-D-deoxyribosyl sugar moiety. A subscript “k” indicates a cEt. A subscript “s” indicates a phosphorothioate internucleoside linkage. [_{α-D}B_{ds}] indicates a modified, α-D-DNA nucleotide with a 2′-deoxyribosyl sugar moiety, a phosphorothioate linkage, and base B.

For the results in the tables below, in vitro activity and toxicity experiments were performed essentially as described in Example 11. For in vitro activity and toxicity studies, 3T3-L1 cells were transfected with 27 nM, 80 nM, 250 nM, 740 nM, 2, 222 nM, 6,667 nM, or 20,000 nM of modified oligonucleotide by electroporation and levels of p21 were measured by RT-qPCR as described in Example 11 above. The caspase assay was performed as described in Example 11 above in 3T3-L1 cells.

Selected modified nucleotides below were tested for their effect on HeLa cells by microscopy. HeLa cells were transfected by lipofectamine 2000 with 200 nM of modified oligonucleotide for 2 hrs and then cellular protein p54nrb was stained by mP54 antibody (Santa Cruz Biotech, sc-376865) and DAPI was used to stain for the nucleus of cells. The number of cells with nucleolar p54nrb and the total number of cells in the images were counted.

TABLE 25
In vitro activity and toxicity of modified oligonucleotides
complementary CXCL12

| | | in vitro | | |
|---|---|---|---|---|
| | in vitro | Caspase | in vitro p21 | |
| Compound | CXCL12 | (% control) @ | (% control) | % nucleolar |
| ID | IC₅₀ (nM) | 20 μM | @ 20 μM | p54nrb |
| 1244458 | 19 | 785 | 327 | 86 |
| 1244448 | 35 | 269 | 135 | 66 |
| 1244449 | 169 | 111 | 101 | 8 |
| 1244450 | 103 | 96 | 169 | 11 |
| 1244451 | 45 | 261 | 206 | 78 |
| 1244452 | 393 | 295 | 146 | 83 |
| 1244453 | 80 | 417 | 255 | 92 |
| 1244454 | 512 | 287 | 240 | 65 |
| 1244455 | 125 | 409 | 310 | 83 |
| 1244456 | 247 | 233 | 269 | 96 |
| 1244457 | 31 | 854 | 400 | 100 |

### Example 14: 4′-Methyl Stereo-Standard Nucleosides or Stereo-Non-Standard 2′Deoxy-β-D-XNA Nucleosides

Modified oligonucleotides containing an altered nucleotide with a 4′-methyl modified sugar moiety or a stereo-non-standard 2′-deoxy-β-D-xylofuranosyl (2′deoxy-β-D-XNA) sugar moiety at various positions were synthesized using standard techniques or those described herein (see Table 26 below). Synthesis of oligonucleotides comprising 2′deoxy-β-D-XNA nucleosides has been described previously (Wang, et. al., Biochemistry, 56(29): 3725-3732, 2017). Synthesis of oligonucleotides comprising 4′-methyl modified nucleosides has been described previously (e.g., Detmer et. al., European J. Org. Chem, 1837-1846, 2003). The compounds in Table 26 each comprise a 5′ wing and a 3′ wing each consisting of three linked cEt nucleosides and a central region comprising nucleosides each comprising 2′-β-D-deoxyribosyl sugar moieties aside from the altered nucleotide, as indicated. Each internucleoside linkage is a phosphodiester internucleoside linkage. These compounds were compared to a compound comprising a 2′-OMe modified sugar moiety at position 2 of the central region, 936053. The compounds in the table below are 100% complementary to mouse CXCL12, GENBANK NT_039353.7 truncated from 69430515 to 69445350 (SEQ ID NO: 1), at position 6877 to 6892.

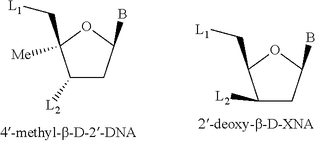

A 2′deoxy-β-D-XNA is a nucleoside of Formula II, wherein J₃ and J₄ are each H.

TABLE 26
modified oligonucleotides with stereochemical modifications

| | position | | | |
|---|---|---|---|---|
| | of altered | | | |
| | nucleotide | modification | | SEQ |
| Compound | in central | of altered | | ID |
| Number | region | nucleotide | Chemistry Notation | NO |
| 936053 | 2 | 2′-OMe | Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | 5 |
| 1244461 | 3 | 4′-Me | Gₖₛ^{m}CₖₛAₖₛT_{ds}G_{ds}T_{[4m]s}T_{ds}^{m}C_{ds}T_{ds}^{m}C_{ds}A_{ds}^{m}C_{ds}A_{ds}TₖₛTₖₛAₖ | 5 |
| 1244462 | 4 | 4′-Me | Gₖₛ^{m}CₖₛAₖₛT_{ds}G_{ds}T_{ds}T_{[4m]s}^{m}C_{ds}T_{ds}^{m}C_{ds}A_{ds}^{m}C_{ds}A_{ds}TₖₛTₖₛAₖ | 5 |
| 1263776 | 3 | β-D-XNA | Gₖₛ^{m}CₖₛAₖₛT_{ds}G_{ds}[_{β-D}Tₓₛ]T_{ds}^{m}C_{ds}T_{ds}^{m}C_{ds}A_{ds}^{m}C_{ds}A_{ds}TₖₛTₖₛAₖ | 5 |
| 1263777 | 4 | β-D-XNA | Gₖₛ^{m}CₖₛAₖₛT_{ds}G_{ds}T_{ds}[_{β-D}Tₓₛ]^{m}C_{ds}T_{ds}^{m}C_{ds}A_{ds}^{m}C_{ds}A_{ds}TₖₛTₖₛAₖ | 5 |

A subscript “d” indicates an unmodified, 2′-β-D-deoxyribosyl sugar moiety. A subscript “k” indicates a cEt. A subscript “s” indicates a phosphorothioate internucleoside linkage. A superscript “m” indicates 5-methyl Cytosine. A subscript “[4m]” indicates a 4′-methyl-2′-β-D-deoxyribosyl sugar moiety. [_{β-D-}Bₓₛ] indicates a modified, β-D-XNA (xylo) nucleotide with a 2′-deoxyxylosyl sugar moiety, a phosphorothioate linkage, and base B.

For in vivo activity and toxicity studies, 3 BALB/c mice per group were administered 10 or 150 mg/kg modified oligonucleotide by subcutaneous injection and sacrificed after 72 hours. Four animals were administered saline to serve as a control. RT-PCR was performed as described in Example 11 to determine mRNA levels of CXCL12, P21, Tnfrsf10b, and Gadd45a. Plasma levels of ALT was measured using an automated clinical chemistry analyzer. Increased ALT is indicative of acute liver toxicity.

TABLE 27
In vivo activity and toxicity of modified oligonucleotides complementary to CXCL12

| | in vivo | in vivo | in vivo | in vivo | in vivo | in vivo | in vivo |
|---|---|---|---|---|---|---|---|
| | CXCL12 @ | CXCL12 @ | P21 @ | Tnfrsf10b @ | Gadd45a @ | ALT @ | ALT @ 150 |
| Compound | 10 mg/kg | 150 mg/kg | 150 mg/kg | 150 mg/kg | 150 mg/kg | 10 mg/kg | mg/kg |
| ID | (% control) | (% control) | (% control) | (% control) | (% control) | (IU/L) | (IU/L) |
| PBS | 100 | 100 | 100 | 100 | 100 | 26 (@ 0 mg/kg) | |
| 936053 | 37 | 13 | 175 | 448 | 216 | 23 | 83 |
| 1244461 | 22 | 5 | 2994 | 4663 | 1124 | 31 | 5080 |
| 1244462 | 30 | 7* | 1038 | 717* | 407* | 28 | 1789* |
| 1263776 | 19 | 11 | 4846 | 10686 | 1032 | 27 | 9234 |
| 1263777 | 13 | n.d. | n.d. | n.d. | n.d. | 58 | death |

*Value represents the average of 2 samples.

### Example 15: Exonuclease Stability of Stereo-Non-Standards Nucleosides

Oligonucleotides comprising stereo-standard and stereo-nonstandard nucleosides were synthesized using standard techniques or those described herein. Each oligonucleotide in the table below has the sequence TTTTTTTTTTTT (SEQ ID NO: 10) or TTTTTTTTTTUU (SEQ ID NO: 11) and has a full phosphodiester backbone. For each compound other than the DNA control, the two 3′ terminal nucleosides are modified nucleosides as indicated in the table below.

TABLE 28
Design of Compounds

| Compound | | SEQ | Modification of 3′ terminal |
|---|---|---|---|
| ID | Chemistry Notation | ID NO | nucleosides |
| 7157 | T_{do}T_{do}T_{do}T_{do}T_{do}T_{do}T_{do}T_{do}T_{do}T_{do}T_{do}T_{d} | 10 | Unmodified DNA (control) |
| 395421 | T_{do}T_{do}T_{do}T_{do}T_{do}T_{do}T_{do}T_{do}T_{do}T_{do}TₑₒTₑ | 10 | 2′-MOE (control) |
| 395423 | T_{do}T_{do}T_{do}T_{do}T_{do}T_{do}T_{do}T_{do}T_{do}T_{do}UₗₒUₗ | 11 | 2′-4′-LNA (control) |
| 1427914 | T_{do}T_{do}T_{do}T_{do}T_{do}T_{do}T_{do}T_{do}T_{do}T_{do}[_{α-L}Tₘₒ][_{α-L}Tₘ] | 10 | α-L-2′-OMe-DNA |
| 1427915 | T_{do}T_{do}T_{do}T_{do}T_{do}T_{do}T_{do}T_{do}T_{do}T_{do}[_{β-L}T_{do}][_{β-L}T_{d}] | 10 | β-L-DNA |
| 1427916 | T_{do}T_{do}T_{do}T_{do}T_{do}T_{do}T_{do}T_{do}T_{do}T_{do}[_{β-D}Tₓₒ][_{β-D}Tₓ] | 10 | β-D-XNA |
| 1427917 | T_{do}T_{do}T_{do}T_{do}T_{do}T_{do}T_{do}T_{do}T_{do}T_{do}[_{α-L}T_{do}][_{α-L}T_{d}] | 10 | α-L-DNA |
| 1427918 | T_{do}T_{do}T_{do}T_{do}T_{do}T_{do}T_{do}T_{do}T_{do}T_{do}[_{α-D}T_{do}][_{α-D}T_{d}] | 10 | α-D-DNA |

A subscript “d” indicates a nucleoside comprising an unmodified, 2′-β-D-deoxyribosyl sugar moiety.
A subscript “l” indicates a LNA.
A subscript “o” indicates a phosphodiester internucleoside linkage.
[_{α-L}Tₘₒ] indicates a stereo-non-standard α-L-2′-OMe-DNA nucleotide with a 2′-OMe-deoxyribosyl sugar moiety, a phosphodiester internucleoside linakge, and base T.
[_{β-L}T_{do}] indicates a stereo-non-standard α-D-DNA nucleotide with a 2′-deoxyribosyl sugar moiety, a phosphodiester internucleoside linkage, and base T.
[_{β-D}Tₓₒ] indicates a stereo-non-standard β-D-XNA nucleotide with a 2′-deoxyxylosyl sugar moiety, a phosphodiester internucleoside linkage, and base T.
[_{α-L}T_{do}] indicates a stereo-non-standard α-L-DNA nucleotide with a 2′-deoxyribosyl sugar moiety, a phosphodiester internucleoside linkage, and base T.
[_{α-D}T_{do}] indicates a stereo-non-standard α-D-DNA nucleotide with a 2′-deoxyribosyl sugar moiety, a phosphodiester internucleoside linkage, and base T.

The oligonucleotides described above were incubated at 5 μM concentration in buffer with snake venom phosphodiesterase (SVPD, Sigma P4506, Lot #SLBV4179), a strong 3′-exonuclease, at the standard concentration of 0.5 mU/mL and at a higher concentration of 2 mU/mL. SVPD is commonly used to measure the stability of modified nucleosides (see, e.g., Antisense Drug Technology, Crooke S. T., Ed., CRC Press, 2008). Aliquots were removed at various time points and analyzed by MS-HPLC with an internal standard. Relative peak areas were plotted versus time and half-life was determined using PrismGraphPad. A longer half-life means the 3′-terminal nucleosides have increased resistance to the SVPD exonuclease. The results show that stereo-non-standard DNA isomers are significantly more stable to exonuclease degredatation than unmodified DNA, and several stereo-non-standard DNA isomers are significantly more stable than 2′-MOE or 2′-4′-LNA modified DNA.

TABLE 29
Exonuclease resistance of stereo-non-standard nucleosides

| | | Half-life of | |
|---|---|---|---|
| | | oligonucleotides in SVPD | |
| | | (minutes) | |
| | | | 20 |
| Compound | Modification of 3′ | 5 units/mL | units/mL |
| ID | terminal nucleosides | 0-30 min | 0-120 min |
| 7157 | DNA (control) | 0.4 | N/A |
| 395421 | 2′-MOE (control) | 7.1 | 1.2 |
| 395423 | 2′-4′-LNA (control) | 3.2 | 0.9 |
| 1427914 | α-L-2′-OMe-DNA | >30 | >120 |
| | | | (210 est.) |
| 1427915 | β-L-DNA | >30 | 28.7 |
| 1427916 | β-D-XNA | >30 | 7.1 |
| 1427917 | α-L-DNA | >30 | 70.1 |
| 1427918 | α-D-DNA | 4.0 | N/A |

### Example 16: Design and Synthesis of Stereo-Non-Standard Nucleosides and 2′-Substituted Stereo-Non-Standard Nucleosides

2′-substituted stereo-non-standard nucleosides and stereo-non-standard nucleosides described herein were prepared as amidites as described below. The stereo-non-standard nucleoside amidites may then be incorporated into a modified oligonucleotide during modified oligonucleotide synthesis.

Compound 12, an amidite of a stereo-non-standard nucleoside, was prepared according to the scheme below:

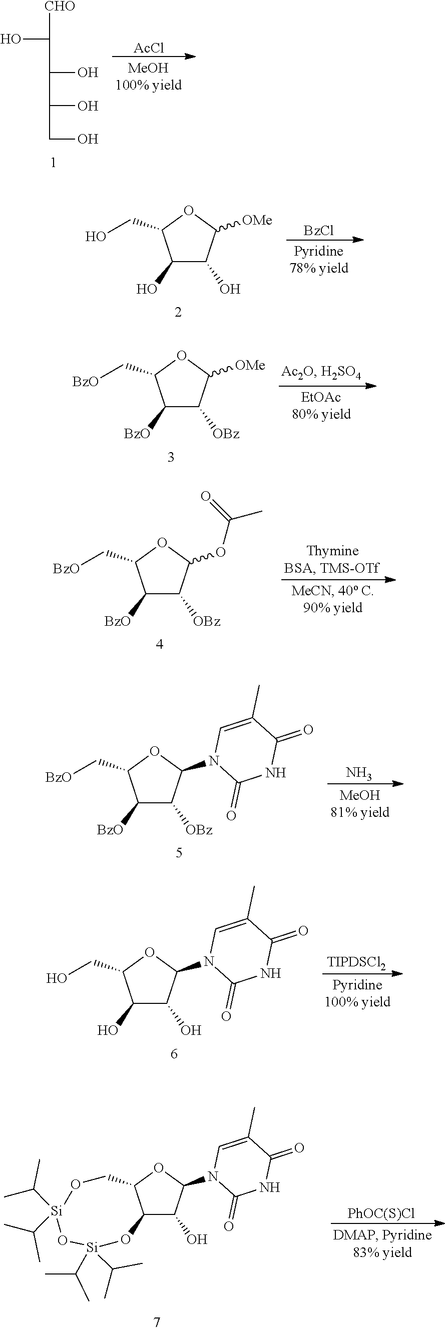

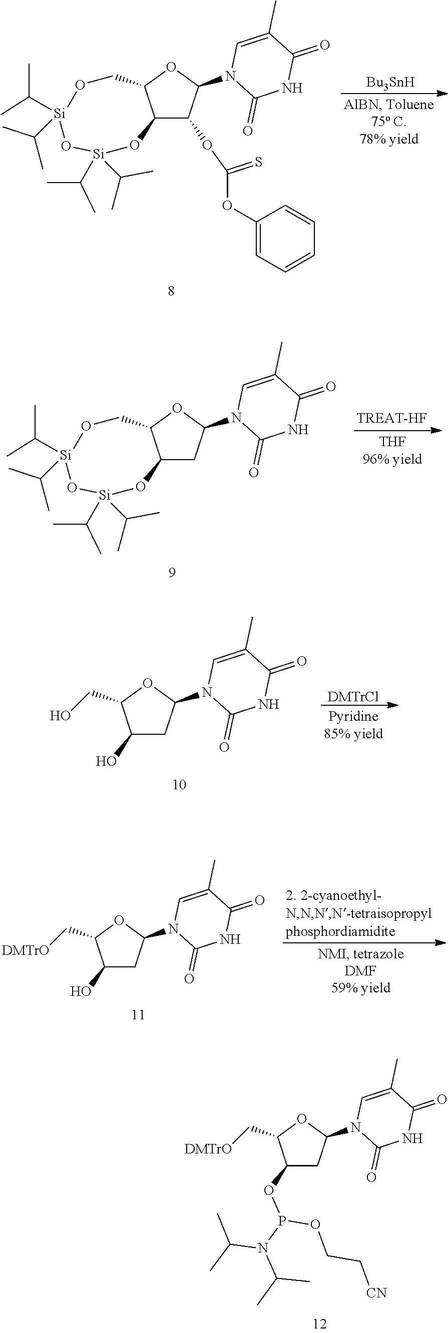

### Synthesis of Final Compound 12.

Compound 1 was obtained from a commercial supplier.

Compound 2. Acetyl chloride (13.3 M, 2.50 mL, 33.3 mmol) was added dropwise to methanol (30.0 mL) at 0° C. The resultant methanolic hydrogen chloride solution was then added slowly to a solution of 2,3,4,5-tetrahydroxypentanal (compound 1, 1.00 g, 6.66 mmol) in methanol (100 mL). After 3 hours of stirring at room temperature the reaction was neutralized by addition of pyridine (20 mL) and evaporated to provide the desired compound as an oil. Dried under high vacuum overnight and used in next step with no further purification.

Compound 3. Compound 2 (5.47 g, 33.3 mmol) was dissolved in Pyridine (40.00 mL) and cooled to 0° C. Benzoyl chloride (31.0 mL, 267 mmol) was added slowly. The reaction was warmed to room temperature and stirred overnight. Water was then added and the reaction mixture was extracted with dichloromethane. The combined organic extracts were washed with 10% hydrochloric acid (aq), (3×300 mL) and evaporated under reduced pressure. The crude reaction mixture was purified by Biotage (Si, 220 g col, 0-20% Ethyl acetate/Hexanes) to give the desired product as a clear colorless oil. (12.4 g, 26.0 mmol, yield: 78.1%)

Compound 4. Compound 3 (15.9 g, 33.4 mmol) was dissolved in ethyl acetate (95.0 mL). Acetic anhydride (10.3 mL, 110 mmol) was added followed by sulfuric acid (0.356 mL, 6.67 mmol). After 3 hours stirring at room temperature the reaction was diluted with saturated aqueous sodium bicarbonate solution (100 mL) and ethyl acetate (100 mL). The aqueous layer was extracted with ethyl acetate. The combined organics were washed with saturated sodium bicarbonate solution (aq), water and brine, followed by concentration under reduced pressure to give a crude oil. Purification by Biotage (Si, 10 g col, 0-20% Ethyl acetate/Hexanes) afforded the desired product as white foam. (13.5 g, 26.8 mmol, yield: 80.4%)

Compound 5. Thymine (0.440 g, 3.49 mmol) and N,O-Bis(trimethylsilyl)acetamide (2.33 mL, 9.54 mmol) were added to a solution of compound 4 (1.60 g, 3.17 mmol) in acetonitrile (16.0 mL). After heating at 40° C. for 15 minutes to obtain a clear solution trimethylsilyl trifluoromethanesulfonate (0.746 mL, 4.12 mmol) was added and the reaction was stirred overnight at 40° C. The reaction was concentrated under reduced pressure and diluted with ethyl acetate. The organics were washed with saturated sodium bicarbonate solution and brine, followed by concentration to an oil under reduced pressure. Purification by Biotage (Si, 100 g col, 0-50% Ethyl acetate/Hexanes) afforded the desired product as a white solid. (1.63 g, 2.86 mmol, yield: 90.1%)

Compound 6. NH₃ (7.00 M, 8.26 mL, 57.8 mmol) in methanol was added to a solution of Compound 5 (11.0 g, 19.3 mmol) was dissolved in methanol (80.0 mL). The reaction was heated at 40° C. for 16 hours and then stirred at room temperature for 72 hours. The reaction was concentrated to an oil and purification by Biotage (Si, 25 g col, 0-20% Methanol/Dicholormethane) afforded the desired product as a white solid. (4.05 g, 15.7 mmol, yield: 81.3%)

Compound 7. Compound 6 (3.92 g, 15.2 mmol) was dissolved in pyridine (50 mL) and evaporated to dryness under reduced pressure at 60° C. three times to dry the starting material. This was then dissolved in dry pyridine (50.5 mL) and 1,3-dichloro-1,1,3,3-tetraisopropyldisiloxane (5.83 mL, 18.2 mmol) was added dropwise. The reaction was stirred at room temperature for 30 min. and then concentrated to an oil under reduced pressure. The oil was dissolved in ethyl acetate and the organics were washed with 10% HCl (aq), water, saturated sodium bicarbonate solution, water, brine and concentrated to afford the desired product as a white amorphous solid. (7.61 g, 15.2 mmol, yield: 100%)

Compound 8. Compound 7 (2.84 g, 0.00567 mol) and 4-dimethylaminopyridine (1.39 g, 0.0113 mol) were dissolved in anhydrous acetonitrile (56.8 mL) followed by slow addition of O-4-methylphenyl chlorothioformate (0.951 mL, 0.00624 mol). The reaction was stirred at room temperature for 72 hours. The solvents were removed under reduced pressure and the residue was partitioned between ethyl acetate and water. The aqueous layer was extracted with ethyl acetate and the combined organics were washed with 10% HCl(aq), water, saturated sodium bicarbonate solution, water and brine. The organic fractions were dried over magnesium sulfate and concentrated. Purification by Biotage (Si, 100 g col, 0-40% Ethyl acetate/Hexanes) afforded the desired product as a white solid. (3.06 g, 0.00470 mol, yield: 82.9%)

Compound 9. Azobisisobutyronitrile (AIBN) (0.0101 g, 0.0615 mmol) and Tributyltin hydride (0.894 g, 3.07 mmol) in Toluene (2 mL) were added drop-wise to a degassed (with nitrogen) solution of Compound 8 (0.200 g, 0.307 mmol) in Toluene (4 mL) held at 80° C. The solution continued at 80° C. for 1 hour before being cooled to room temperature and removal of the solvents under reduced pressure. Purification by Biotage (Si, 50 g col, 0-40% Ethyl acetate/Hexanes) afforded the desired product as a white solid (0.116 g, 0.239 mmol, yield: 77.9%)

Compound 10. Triethylamine (0.0812 mL, 0.583 mmol) was added to a solution of compound 9 (0.113 g, 0.233 mmol) in THF (1.16 mL). The reaction was cooled to 0° C. with an ice bath under an atmosphere of nitrogen. Triethylamine trihydrofluoride (0.190 mL, 1.17 mmol) was added slowly at 0° C. and then the reaction was warmed to room temperature and stirred for 1.5 hours. The solvents were removed under reduced pressure and purification by Biotage (Si, 10 g col, 0-10% methanol/dichlormethane) afforded the desired product as a white gummy solid. (54.0 mg, 0.000223 mol, yield: 95.6%)

Compound 11. DMT-Cl (73.9 mg, 0.218 mmol) was added to a solution of 1-[(2R,4R,5S)-4-hydroxy-5-(hydroxymethyl)tetrahydrofuran-2-yl]-5-methyl-pyrimidine-2,4-dione (732 mg, 3.02 mmol) in Pyridine (10.1 mL) at room temperature and stirred for 2 hours. The reaction was quenched with the addition of methanol (0.5 mL), followed by dilution of the reaction with water and ethyl acetate. The aqueous layer was extracted with ethyl acetate. The combined organics were washed with water, saturated sodium bicarbonate and brine. Followed by removal of the solvents under reduced pressure. Purification by Biotage (Si, 100 g col, 0-80% ethyl acetate/hexanes) afforded the desired product as a white solid. (1394 mg, 2.56 mmol, yield: 84.7%)

Compound 12. 1H-Tetrazole (0.157 g, 2.25 mmol) and 1-Methylimidazole (0.0557 mL, 0.702 mmol) were added to a solution of compound 11 (1.53 g, 2.81 mmol) in DMF (22.3 mL) at room temperature under an atmosphere of nitrogen 2-Cyanoethyl N,N,N′,N′-tetraisopropylphosphorodiamidite (1.34 mL, 4.21 mmol) was then added drop-wise and the reaction was stirred at room temperature for 90 minutes. Water (1.0 mL) was added to quench the reaction. A 3:1 mixture of toluene/hexanes (80 mL) was added and the organic layer was washed four times with a 3:2 mixture of DMF/H2O (50 mL). The organics were then washed with saturated sodium bicarbonate solution, brine, dried over solid sodium sulfate and concentrated under reduced pressure to a white foam. Purification by Biotage (Si, 50 g col, 0-60% ethyl acetate/hexanes) afforded the desired product as a white amorphous solid. (1.23 g, 1.65 mmol, yield: 58.8%)

Compound 17, an amidite of a stereo-non-standard nucleoside, was prepared according to the scheme below:

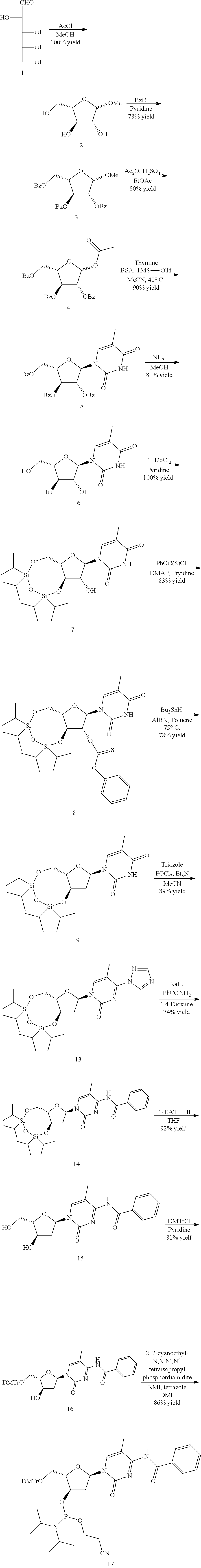

### Synthesis of Final Compound 17.

Compounds 2, 3, 4, 5, 6, 7, 8 and 9 were synthesized as previously described for final compound 12.

Compound 13. POCl₃ (2.53 mL, 27.6 mmol) was added drop-wise to a suspension of 1,2,4-1H-Triazole (7.16 g, 104 mmol) in Acetonitrile (69.0 mL) under an atmosphere of nitrogen at 0° C., followed by drop-wise addition of Triethylamine (19.3 mL, 138 mmol). After 30 minutes at 0° C. a solution of Compound 9 (3.35 g, 6.91 mmol) in THF (10.00 mL) was added drop-wise. This was stirred at room temperature overnight. The reaction was concentrated to small volume under reduced pressure, diluted with ethyl acetate and the organic layer was washed with aqueous saturated sodium bicarbonate (2×), water, brine and concentrated to a yellow oil. Purification by column on Biotage (Si, 25 g col, 0-60% ethyl acetate/hexanes) afforded the desired product as a white amorphous solid. (3.29 g, 6.14 mmol, yield: 88.9%)

Compound 14. 1,4-Dioxane (1.96 mL) was added to NaH (60.0%, 63.3 mg, 1.58 mmol) in a flask under an atmosphere of nitrogen at room temperature. A suspension of Benzamide (192 mg, 1.58 mmol) in 1,4-Dioxane (1.00 mL) was added to the flask and the reaction was stirred for 1 hour at room temperature. A solution of compound 13 (212 mg, 0.396 mmol) in 1,4-dioxane (1.00 mL) was added to the reaction flask and the reaction was stirred for 2 hours at room temperature. The reaction was quenched by addition of saturated aqueous ammonium chloride solution and the aqueous layer was extracted with ethyl acetate. The combined organics were washed with brine, dried over magnesium sulfate and concentrated to a crude solid. Purification by column on Biotage (Si, 25 g col, 0-10% ethyl acetate/hexanes) afforded the desired product as a white solid. (173 mg, 0.294 mmol, yield: 74.4%)

Compound 15. Triethylamine (1.96 mL, 14.0 mmol) was added to a solution of compound 14 (3.30 g, 5.61 mmol) in tetrahydrofuran (56.0 mL). The reaction was cooled to 0° C. under an atmosphere of nitrogen. Triethylamine trihydrofluoride (4.58 mL, 28.1 mmol) was added slowly and then the reaction was warmed to room temperature with stirring for 3 hours. The solvents were removed under reduced pressure and purification by Biotage (Si, 220 g col, 0-10% methanol/dichlormethane) afforded the desired product as a white solid (1.78 g, 5.15 mmol, yield: 91.7%)

Compound 16. DMT-Cl (1.92 g, 5.66 mmol) was added to a solution of compound 15 (1.78 g, 5.15 mmol) in pyridine (17.1 mL). The reaction was stirred at room temperature for 2 hours. The reaction was quenched with the addition of methanol (0.5 mL), followed by dilution with water and ethyl acetate. The aqueous layer was extracted with ethyl acetate. The combined organics were washed with water, saturated sodium bicarbonate, brine and concentrated under reduced pressure. Purification by Biotage (Si, 220 g col, 0-60% ethyl acetate/hexanes) afforded the desired product as a pale yellow solid. (2.70 g, 4.17 mmol, yield: 81.0%)

Compound 17. 1H-Tetrazole (0.234 g, 3.33 mmol) and 1-Methylimidazole (0.0827 mL, 1.04 mmol) were added to a solution of compound 16 (2.70 g, 4.17 mmol) in DMF (41.6 mL), followed by drop-wise addition of 2-Cyanoethyl N,N,N′,N′-tetraisopropylphosphorodiamidite (1.99 mL, 6.25 mmol) and stirred at room temperature for 90 minutes. Water (1.0 mL) was added to quench the reaction. A 3:1 mixture of toluene/hexanes (80 mL) was added and the organic layer was washed four times with a 3:2 mixture of DMF/H2O (50 mL). The organics were then washed with saturated sodium bicarbonate solution, brine, dried over solid sodium sulfate and concentrated under reduced pressure. Purification by Biotage (Si, 220 g col, 0-50% ethyl acetate/hexanes) (loaded with a small amount of EtOAc) afforded the desired product as a white amorphous solid. (3.03 g, 3.57 mmol, yield: 85.7%)

Compound 26, an amidite of a stereo-non-standard nucleoside, was prepared according to the scheme below:

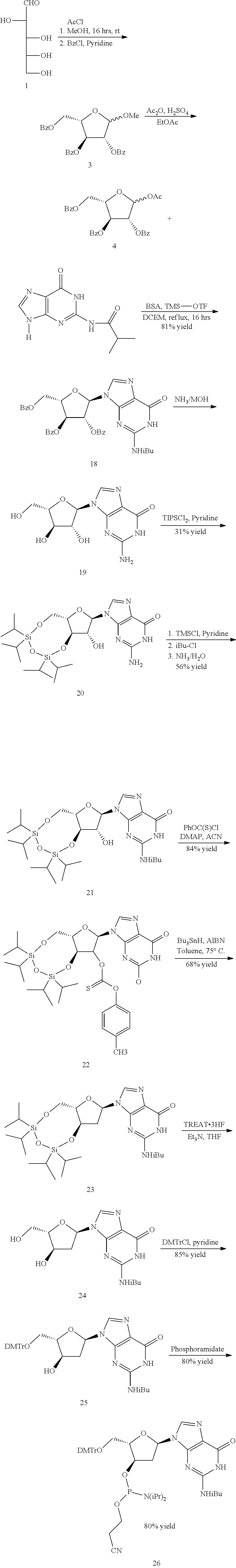

### Synthesis of Final Compound 26.

Compounds 3 and 4 were synthesized as previously described for final compound 12.

Compound 18. 2-(isobutylamino)-1,9-dihydro-6H-purin-6-one and sugar 4, was azeotroped 4× with Toluene at 60° C. The dry 2-(isobutylamino)-1,9-dihydro-6H-purin-6-one (23 g, 119 mmol) and sugar 4 (40 g, 79.3 mmol) was suspend in DCE (800 mL). N,O-Bis(trimethylsilyl)acetamide (75.5 mL, 317 mmol) was added, and the reaction was held at 80° C. for 1 hr. to affect a clear solution. The solution was cooled with an ice bath to 5° C. and trimethylsilyl trifluoromethanesulfonate (23 mL, 127 mmol) was added and the reaction was stirred overnight at 80° C. The next day, the reaction was concentrated under reduced pressure and diluted with ethyl acetate. The organic layer was washed with plain DI water first, then with saturated sodium bicarbonate solution. The organics were then washed with brine, followed by concentration to an oil under reduced pressure. Purification by silica gel glass chromatography (Silica gel 1000 ml 6/4 Diethyl ether/Hexanes) afforded the desired product as a white solid. 43.0 g crude, 81% yield.

Compound 19. Compound 18 (43.0 g, 6560 mmol) was suspend in methanol (50.0 mL) and cooled to −20° C. NH₃/MeOH (7.00 M, 150 mL) was added at 0° C., and the reaction was sealed and heated at 45° C. for 16 hours. The next day, the solution was concentrated to an oil, and then suspended in EtOAc (100 mL) to obtained white precipitate which was collected by filtration and rinsed with fresh EtOAc. Drying the crude solid under high vacuum gave 20 g, 100+% yield. The crude material was azeotroped 3× with pyridine and, without any further purification, was taken to the next step.

Compound 20. Compound 19 (20 g, 76.60 mmol) was dissolved in pyridine (400 mL) under nitrogen, cooled in an ice bath and 1,3-Dichloro-1,1,3,3-tetraisopropyldisiloxane (23.30 mL, 63.60 mmol, 0.90 eq.) was added dropwise. The reaction was allowed to warm up slowly to about 10° C. over 2 hours. TLC in EtOAc/hexane (8/2) indicated reaction was completed. The reaction was quenched by cooling in an ice bath and quenching the reaction by slowly adding DI water (20 mL). About 4 grams of product was collected by filtration, and the remaining solution was concentrated to an oil under reduced pressure. The oil was dissolved in ethyl acetate and the organics were washed with 10% HCl (aq), water, saturated sodium bicarbonate solution, water, brine and concentrated to afford the desired product as a colorless oil. The crude oil was suspended in hexane to obtained additional white solid which was collected by filtration. Final combined weight 14.40 g crude 31% yield.

Compound 21. Compound 20 (14.20 g, 27.10 mmol) was dissolved in pyridine (100 mL) under nitrogen, cooled in an ice bath and then trimethylsilyl chloride (13.20 mL, 135 mmol, 5 eq.) was added dropwise. The ice bath was then removed, and the reaction was stirred for 1 hr at room temperature. The reaction was once again cooled in an ice bath, and isobutyryl chloride (13.40 g, 135 mmol, 5 eq.) was added dropwise. The reaction was allowed to warm up to room temperature and continued to stir overnight. The next day, the reaction was quenched by cooling in an ice bath, and adding water (40 mL), not letting the temperature above 10° C. After an hour, the reaction was cooled yet again and NH₄OH_{(aq)} (55 ml) was added dropwise to the reaction. After stirring for another 30 minutes, the solution was diluted with EtOAc and the organic layer was separated and washed with plain water 100 (ml), sat. NaHCO₃, brine, dried over Na₂SO₄, filtered and evaporated to obtained crude material. The crude material was dissolved and purified by biotage column 100 g, eluted with DCM/MeOH (97/3)+1% Et₃N to obtained 9.0 g, 56% yield.

Compound 22. Compound 21 (7.80 g, 131 mmol) and 4-Dimethylaminopyridine (3.20 g, 262 mmol, 2 eq.) were dissolved in anhydrous Acetonitrile (131 mL). To this was added O-4-Methylphenyl Chlorothioformate (2.69 mL, 144 mmol, 1.2 eq.) dropwise. The reaction was stirred at room temperature for 16 hours. The next day the reaction was deemed to be complete by TLC in DCM/MeOH (95/5). The solvents were removed under reduced pressure and the residue was partitioned between ethyl acetate and water. The aqueous layer was extracted with ethyl acetate and the combined organics were washed with 10% HCl(aq), water, saturated sodium bicarbonate solution, water and brine. The organic fractions were dried over magnesium sulfate and concentrated. Purification by Biotage (Si, 100 g col, eluded with 0-3% Dichloromethane/Methanol) afforded the desired product as a white solid 8.24 g, 84% yield.

Compound 23. Azobisisobutyronitrile (AIBN) (0.267 g, 1.80 mmol, 0.2 eq) and tributyltin hydride (24.10 ml, 89.4 mmol 10 eq.) in toluene (40 mL) were degassed for 30 minutes with nitrogen, and then added dropwise to a degassed (with nitrogen) solution of compound 31 (6.67 g, 8.94 mmol) in toluene (140 mL) preheated to 80° C. The solution continued at 80° C. for 1 hour before being cooled to room temperature and removing the solvents under reduced pressure. Purification by Biotage (Si, 100 g col, 70% Ethyl acetate/Hexanes) afforded the desired product as a white solid. 3.54 g, 68% yield.

Compound 24. Triethylamine (2.13 mL, 15.40 mmol, 2.5 eq.) was added to a solution of compound 23 (3.54 g, 6.11 mmol) in THF (30 mL). The reaction was cooled to 0° C. with an ice bath under an atmosphere of nitrogen, and triethylamine trihydrofluoride (4.98 mL, 30.5 mmol, 5 eq.) was added slowly at 0° C. After the addition was complete, the reaction allowed to proceed at room temperature for 16 hours. The solvents were removed under reduced pressure and purification by a plug of silica gel 50 g, eluting with 5-10% methanol/dichlormethane) afforded the desired product as a white solid. 3.7 g, 100+% yield. Product has significant amount of TREAT.3HF that is hard to remove. Without any further purification crude material was taken for the next step. This requires the need for excess DMT-Cl.

Compound 25. DMT-Cl (4.36 g, 13.20 mmol, 1.2 eq.) was added to a solution of compound 24 (3.70 g, 110 mmol) in pyridine (30 mL) at room temperature and then stirred for 2 hours. The reaction was then quenched with the addition of methanol (2 mL), followed by dilution of the reaction with water and ethyl acetate. The aqueous layer was further extracted with ethyl acetate. The combined organics were washed with water, saturated sodium bicarbonate and brine. The organic layer was separated and dried over Na₂SO₄, filtered, and evaporated under reduced pressure to obtain crude product. This was dissolved in DCM and purified by Biotage (Si, 100 g col, 0-5% Methanol/Dichloromethane) to afford the desired product as a white solid. 3.30 g, 85% yield.

Compound 26. 1H-Tetrazole (0.294 g, 4.25 mmol, 0.8 eq.) and 1-Methylimidazole (0.105 mL, 1.33 mmol, 0.25 eq.) were added to a solution of compound 25 (3.40 g, 5.33 mmol) in DMF (40 mL) at room temperature under an atmosphere of nitrogen. 2-Cyanoethyl N,N,N′,N′-tetraisopropylphosphorodiamidite (2.53 mL, 7.97 mmol, 1.5 eq.) was then added drop-wise and the reaction was stirred at room temperature for 90 minutes. Water (1.0 mL) was added to quench the reaction. A 3:1 mixture of toluene/hexanes (80 mL) was added and the organic layer was washed four times with a 3:2 mixture of DMF/H2O (50 mL). The organics were then washed with saturated sodium bicarbonate solution, brine, dried over solid sodium sulfate and concentrated under reduced pressure to a white foam. Purification by plug of silica gel 50 g, eluded with 100% EtOAc afforded the desired product as a white amorphous solid. 3.54 g, 80% yield.

Compound 35, an amidite of a stereo-non-standard nucleoside, was prepared according to the scheme below:

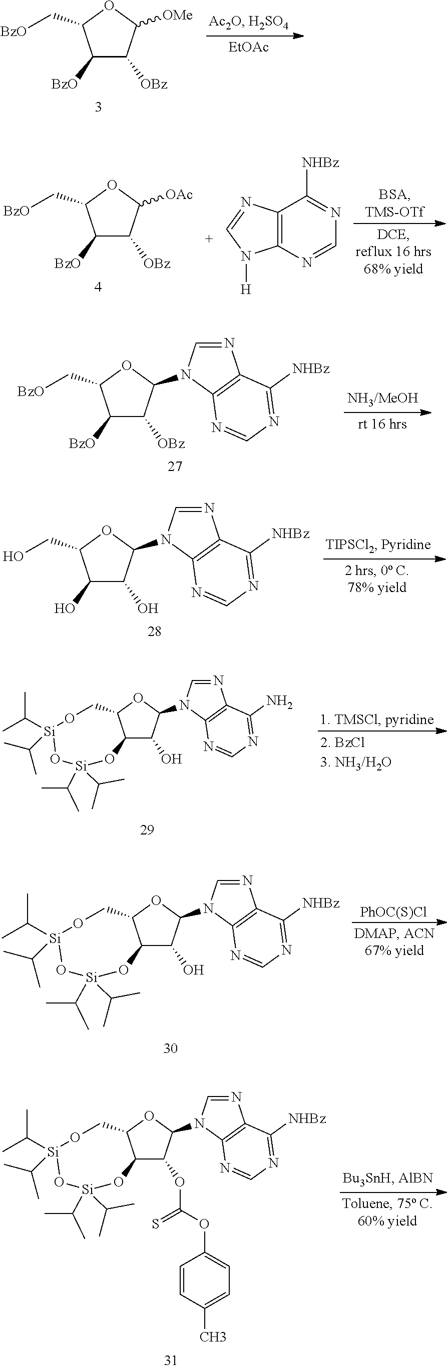

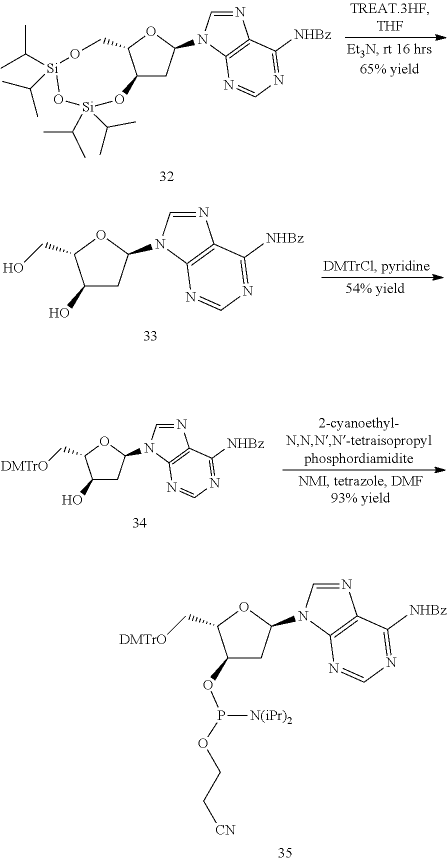

### Synthesis of Final Compound 35.

Compounds 3 and 4 were synthesized as previously described for final compound 12.

Compound 27: N-(9H-purin-6-yl)benzamide and sugar 4, was azeotroped 4× with Toluene at 60° C. Then N-(9H-purin-6-yl)benzamide (23.40 g, 97.30 mmol, 1.30 eq.) and sugar 4 (38 g, 75.3 mmol) were suspend in DCE (800 mL) followed by the addition of N,O-bis(trimethylsilyl)acetamide (73.7 mL, 301 mmol, 4 eq.) After reflux at 80° C. for 1 hr to obtain a clear solution, the reaction solution was cooled with ice bath to 5° C. and trimethylsilyl trifluoromethanesulfonate (21.80 mL, 121 mmol, 1.6 eq.) was added. The reaction was stirred overnight at 80° C. The next day, the reaction was concentrated under reduced pressure and diluted with ethyl acetate. The organic was washed with plain DI water first, then with saturated sodium bicarbonate solution. Washed with brine, followed by concentration to an oil under reduced pressure. Purification by Biotage (Si, 320 g col, eluded with 0-5% Dichloromethane/Methanol) afforded the desired product as a white solid 35.18 g, 68% yield.

Compound 28. Compound 27 (43.0 g, 58.50 mmol) was suspended in methanol (50.0 mL) and cooled to −20° C. NH₃/MeOH (7.00 M, 150 mL) was added, and the reaction was heated at 45° C. for 16 hours in a sealed tube. The next day, the reaction was concentrated to an oil. The crude oil was suspended in EtOAc (100 mL) to obtain a white precipitate, which was collected by filtration and rinsed with EtOAc. Drying the crude solid under high vacuum gave the desired compound 11.70 g, 75% yield. Material was azeotroped 3× with pyridine and was taken to the next step without any further purification.

Compound 29. Compound 28 (11.76 g, 43.78 mmol) was dissolved in pyridine (400 mL) under nitrogen, cooled with ice bath to 0° C. and then 1,3-Dichloro-1,1,3,3-tetraisopropyldisiloxane (12.66 mL, 39.60 mmol, 0.90 eq.) was added dropwise. The reaction was allowed to come to about 10° C. for 2 hours. TLC in EtOAc/hexane (8/2) indicated reaction was completed. The reaction was quenched at 0° C. by slowly adding DI water (20 mL), and then concentrated to an oil under reduced pressure. The oil was dissolved in ethyl acetate and the organics were washed with 10% HCl (aq), water, saturated sodium bicarbonate solution, water, brine and then concentrated to afford the desired product as a colorless oil. Crude oil was suspended in hexane to obtain a white precipitate. Final weight 13.90 g, crude 62% yield.

Compound 30. Compound 29 (7.90 g, 15.50 mmol) was dissolved in pyridine (100 mL) under nitrogen, cooled in an ice bath at 0° C., and trimethylsilyl chloride (13.80 mL, 108 mmol, 5 eq.) was added dropwise. The ice bath was removed and the reaction was allowed to stir at room temperature for 1 hr. The reaction was cooled again in an icebath, and benzoyl chloride (9 mL, 77.50 mmol, 5 eq.) was added dropwise. The reaction was allowed to warm up slowly to rt and continued stirring overnight. The next day, the reaction was cooled with an ice bath and water (150 ml) was added dropwise, keeping the temperature below 7° C. After the addition was completed, the reaction was allowed to stir at room temperature for 1 hour. After cooling the reaction once again to 0° C., NH₄OH_{(aq.)} (100 ml) was added dropwise. After stirring for another 30 minutes, most of the NH₄OH was evaporated at room temperature to obtained mostly water and product. This was diluted with EtOAc and the organic were washed with plain water 100 (ml), sat. NaHCO₃, brine and finally dried over Na₂SO₄, filtered and evaporated to obtain the crude material. The crude material was dissolved in DCM and purified by Biotage (Si, 100 g col, eluded with 0-5% Dichloromethane/Methanol) which afforded the desired product as a white solid 9.20 g, 96% yield.

Compound 31. Compound 30 (8.0 g, 130 mmol) and 4-Dimethylaminopyridine (3.18 g, 261 mmol, 2 eq.) were dissolved in anhydrous Acetonitrile (131 mL) If nucleoside starting material crystallizes out, added some anhydrous THF 50 mL to dissolve. This was followed by slow addition of O-4-Methylphenyl Chlorothioformate (2.18 mL, 143 mmol, 1.2 eq.). The reaction was stirred at room temperature for 16 hours. The next day, reaction was checked by TLC in DCM/MeOH (95/5). The solvents were removed under reduced pressure and the residue was partitioned between ethyl acetate and water. Product was further extracted from aqueous layer with ethyl acetate 2× and the combined organics were washed with 10% HCl(aq), water, saturated sodium bicarbonate solution, water and brine. The organic fraction was dried over magnesium sulfate and concentrated. Purification by Biotage (Si, 100 g col, eluded with 0-3% Dichloromethane/Methanol) afforded the desired product as a white solid 6.67 g, 67% yield.

Compound 32. Azobisisobutyronitrile (AIBN) (0.287 g, 1.75 mmol, 0.2 eq) and Tributyltin hydride (23.50 ml, 87.30 mmol 10 eq.) in Toluene (40 mL), were added dropwise to a degassed (with nitrogen, 30 minutes) solution of compound 22 (6.67 g, 8.73 mmol) in Toluene (140 mL) at 80° C. The solution was heated at 80° C. for 1 hour before being cooled to room temperature and the solvents removed under reduced pressure. The reaction was monitored by TLC in EtOAc/Hexane (7/3). Purification by Biotage (Si, 100 g col, 70% Ethyl acetate/Hexanes) afforded the desired product as a white solid. 3.0 g, 60% yield.

Compound 33. Triethylamine (1.36 mL, 9.80 mmol, 2.5 eq.) was added to a solution of Compound 32 (2.34 g, 3.91 mmol) in THF (30 mL). The reaction was cooled to 0° C. with an ice bath under an atmosphere of nitrogen. Triethylamine Trihydrofluoride (3.19 mL, 20 mmol, 5 eq.) was added slowly at 0° C. and then the reaction was warmed to room temperature and stirred for 16 hours. The solvents were removed under reduced pressure and purified by plug of silica gel 50 g, eluting with 5-10% methanol/dichlormethane) to afford the desired product as a white solid. 0.90 g, 65% yield.

Compound 34. DMTCl (1.1 g, 3.04 mmol, 1.2 eq.) was slowly added to a solution of Compound 33 (0.90 g, 2.53 mmol) in Pyridine (20 mL) at room temperature and stirred for 2 hours. The reaction was quenched with the addition of methanol (2 mL), followed by dilution of the reaction with water and ethyl acetate. The aqueous layer was extracted with ethyl acetate. The combined organics were washed with water, saturated sodium bicarbonate and brine. Organic was dry over Na₂SO₄. The solution was filtered and evaporated to the obtain crude product, which was dissolved in DCM and loaded onto Biotage for purification (Si, 50 g col, 0-5% Methanol/Dichloromethane) to afford the desired product as a white solid. 0.90 g, 54% yield.

Compound 35. 1H-Tetrazole (0.075 g, 1.09 mmol, 0.8 eq.) and 1-Methylimidazole (0.0271 mL, 0.342 mmol, 0.25 eq.) were added to a solution of compound 25 (0.90 g, 1.37 mmol) in DMF (10 mL) at room temperature under an atmosphere of nitrogen. 2-Cyanoethyl N,N,N′,N′-tetraisopropylphosphorodiamidite (0.652 mL, 2.05 mmol, 1.5 eq.) was then added drop-wise and the reaction was stirred at room temperature for 90 minutes. Water (1.0 mL) was added to quench the reaction. A 3:1 mixture of toluene/hexanes (80 mL) was added and the organic layer was washed four times with a 3:2 mixture of DMF/H2O (50 mL). The organics were then washed with saturated sodium bicarbonate solution, brine, dried over solid sodium sulfate and concentrated under reduced pressure to a white foam. Purification by plug of silica gel 30 g, eluded with EtOAc/Hexane (9/1) afforded the desired product as a white amorphous solid. 1.10 g, 93% yield.

Compounds 38 and 43, amidites of stereo-non-standard nucleosides, were prepared according to the scheme below:

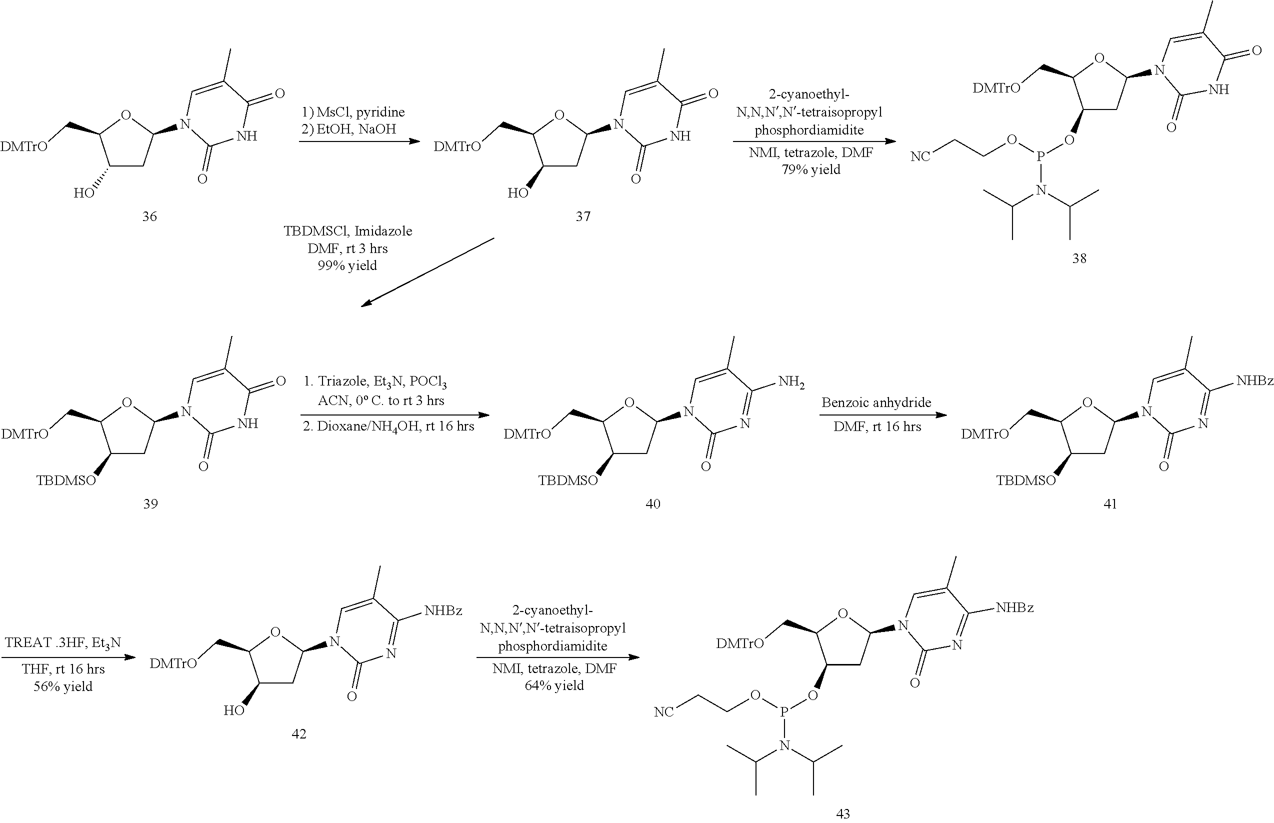

### Synthesis of Final Compound 38 and 43.

Compound 36 was obtained from a commercial supplier.

Compound 37. Has been prepared from compound 36 many times previously. Some examples:
Meyer, A.; et al: Chemical Communications (Cambridge, United Kingdom) (2015), 51(68), 13324-13326
Martin, S. J.; et al Nuclear Medicine and Biology (2002), 29(2), 263-273
Kong, Jong Rock; et al Nucleosides, Nucleotides & Nucleic Acids (2001), 20(10 & 11), 1751-1760

Compound 38. 1H-Tetrazole (0.5647 g, 7.92 mmol 0.8 eq.) and 1-Methylimidazole (0.196 mL, 2.47 mmol, 0.25 eq) were added to a solution of Compound 37 1-((2R,4R,5R)-5-((bis(4-methoxyphenyl)(phenyl)methoxy)methyl)-4-hydroxytetrahydrofuran-2-yl)-5-methylpyrimidine-2,4(1H,3H)-dione (5.31 g, 9.90 mmol) in DMF (51 mL) at room temperature under an atmosphere of nitrogen. 2-Cyanoethyl N,N,N′,N′-tetraisopropylphosphorodiamidite (4.72 mL, 14.80 mmol, 1.5 eq.) was then added drop-wise and the reaction was stirred at room temperature for 90 minutes. Water (1.0 mL) was added to quench the reaction. A 3:1 mixture of toluene/hexanes (80 mL) was added and the organic layer was washed four times with a 3:2 mixture of DMF/H2O (50 mL). The organics were then washed with saturated sodium bicarbonate solution, brine, dried over solid sodium sulfate and concentrated under reduced pressure to obtained crude oil. Crude material, was dissolved in DCM+1% Et₃N and loaded into a plug of silica gel (50 g). The silica gel was first treated with EtOAc/hexane (1/1)+1% Et₃N, before material was loaded. Eluted with EtOAc/hexane (1/1)+1% Et₃N to obtained 5.80 g, 79% yield.

Compound 39. Compound 37, 1-[(2R,3R,4R,5S)-3,4-dihydroxy-5-(hydroxymethyl)tetrahydrofuran-2-yl]-5-methyl-pyrimidine-2,4-dione (4.56 g, 8.37 mmol) was dissolved in anhydrous Dimethylformamide (40 mL) and the solution was stirred under nitrogen. 1H-imidazole (1.44 g, 16.7 mmol, 2 eq.) was added; solution was cooled with icebath at 0° C. and tert-butylchlorodimethylsilane (1.40 g, 16.7 mmol, 2 eq) was added dropwise in a solution of anhydrous dimethylformamide (10 mL). Removed the icebath and let reaction warm up to room temperature and continued stirring for 3 hours. TLC in hexane/EtOAc (6/4) indicated reaction was completed. Cooled solution with icebath to 0° C., and slowly quenched reaction by adding 30 ml of water. Transferred solution to a separatory funnel, and washed with plain DI water and extracted product with ethyl acetate. Removed aqueous layer from the organic and continued to wash the organic with sat. NaHCO₃ and brine, dried over Na₂SO₄, filtered and evaporated solvent to obtain crude oil. The crude material was dissolved in dichloromethane and loaded onto a plug of silica gel and eluted with EtOAc/hexane (6/4) to obtain compound 39, 5.50 g, 99% yield.

Compound 40. POCl₃ (6.45 mL, 70.40 mmol, 8 eq) was added drop-wise to a suspension of 1,2,4-1H-Triazole (20.7 g, 299 mmol, 34 eq.) in acetonitrile (200 mL) under an atmosphere of nitrogen at 0° C. After the addition, the ice bath was removed and the reaction was stirred at room temperature for 20 minutes. The reaction was cooled down again to 0° C. and triethylamine (49.10 mL, 352 mmol, 40 eq.) was added by drop-wise. Compound 39 (5.80 g, 8.80 mmol) in acetonintrile (20 mL) was added drop-wise. This was stirred at room temperature overnight. The reaction was concentrated to small volume under reduced pressure, diluted with ethyl acetate and the organic layer was washed with aqueous saturated sodium bicarbonate (2×), water, brine and concentrated to a yellow oil to afford the desired crude material. The crude material was suspended in Dioxane/NH₄OH_{(aq)} (30 mL/10 mL) solution and stirred at room temperature for 2 hours. TLC in EtOAc/hexane (8/2) indicated reaction was completed. Solvent was concentrated under reduced pressure and remaining oil was diluted with ethyl acetate and washed with 1×200 ml plain DI water and 1×200 ml sat. NaHCO₃. The organic layer was dried over Na₂SO₄, filtered and concentrated under reduced pressure to obtain a crude oil. The crude material was dissolved in DCM and loaded onto a plug of silica gel and eluted with Dichloromethane/Methanol (95/5) to obtain 5.0 g of crude material (product+unreacted starting material).

Compound 41. Compound 40 4-amino-1-((2R,4R,5R)-5-((bis(4-methoxyphenyl)(phenyl)methoxy)methyl)-4-((tert-butyldimethylsilyl)oxy)tetrahydrofuran-2-yl)-5-methylpyrimidin-2(1H)-one (5.30 g, 8.03 mmol) was dissolved in anhydrous dimethylformamide (30 mL) and stirred under nitrogen at room temperature. Benzoic anhydride (2.0 g, 8.83 mmol, 1.1 3 q.) was then added. The reaction was stirred at room temperature overnight. The next day TLC in EtOAc/Hexane (6/4) indicated reaction was completed. Cooled down reaction with ice bath at 0° C. and slowly added about 20 ml of water followed by addition of EtOAc. The mixture was stirred for 10 minutes. The mixture was then transferred to a separatory funnel and washed with plain DI water. The aqueous layer was removed and the organic layer was washed with sat. NaHCO₃ and sat. NaCl. The organic layer was dried over Na₂SO₄ for 10 minutes then the salts were removed by filtration, and the solvent was concentrated under reduced pressure to obtain a crude oil. This was dissolved in DCM and loaded onto plug of SG and eluted with Hexane/EtOAc (6/4). The fractions with product were combined and concentrated under reduced pressure to obtain 1.50 g of pure product and 2.0 grams of compound 39.

Compound 42. Triethylamine (0.88 mL, 6.36 mmol, 2.5 eq.) was added to a solution of compound (41) N-(1-((2R,4R,5R)-5-((bis(4-methoxyphenyl)(phenyl)methoxy)methyl)-4-((tert-butyldimethylsilyl)oxy)tetrahydrofuran-2-yl)-5-methyl-2-oxo-1,2-dihydropyrimidin-4-yl)benzamide (1.50 g, 2.89 mmol) in tetrahydrofuran (10.0 mL). The reaction was cooled to 0° C. under an atmosphere of nitrogen and triethylamine trihydrofluoride (TREAT-HF, 2.08 mL, 12.77 mmol, 5 eq.) was added slowly, afterwards the reaction was allowed to warm to room temperature with stirring for 16 hours. The solvents were removed under reduced pressure and purification by Biotage (Si, 20 g col, 70% EtOAc/Hexane) afforded the desired product as a white solid. 0.66 g, 52% yield.

Compound 43. 1H-Tetrazole (0.0561 g, 0.813 mmol, 0.8 eq.) and 1-Methylimidazole (0.0201 mL, 0.254 mmol) were added to a solution of compound 42 N-(1-((2R,4R,5R)-5-((bis(4-methoxyphenyl)(phenyl)methoxy)methyl)-4-hydroxytetrahydrofuran-2-yl)-5-methyl-2-oxo-1,2-dihydropyrimidin-4-yl)benzamide (0.66 g, 1.02 mmol) in DMF (10 mL), followed by drop-wise addition of 2-Cyanoethyl N,N,N′,N′-tetraisopropylphosphorodiamidite (0.484 mL, 1.52 mmol, 1.5 eq.) and stirring at room temperature for 2 hours. Water (1.0 mL) was added to quench the reaction. A 3:1 mixture of toluene/hexanes (20 mL) was added and the organic layer was washed four times with a 3:2 mixture of DMF/H₂O (20 mL). The organics were then washed with saturated sodium bicarbonate solution, brine, dried over solid sodium sulfate and concentrated under reduced pressure. Purification by Biotage (Si, 20 g col, 40% ethyl acetate/hexanes+1% Et₃N) (loaded with a small amount of DCM) afforded the desired product as a white amorphous solid. 0.55 g, 64% yield.

Compound 47, an amidite of a stereo-non-standard nucleoside, was prepared according to the scheme below:

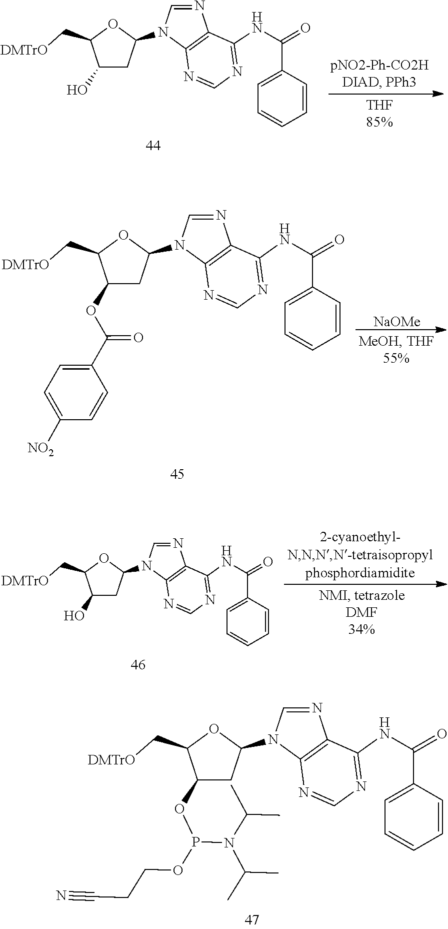

### Synthesis of Final Compound 47.

Compound 44 was obtained from a commercial supplier.

Compound 45. 4-Nitrobenzoic acid (4.07 g, 24.3 mmol) and Triphenyl phosphine (6.38 g, 24.3 mmol) were added to a solution of compound 44, N-[9-[(2R,4S,5R)-5-[[bis(4-methoxyphenyl)-phenyl-methoxy]methyl]-4-hydroxy-tetrahydrofuran-2-yl]purin-6-yl]benzamide (8.00 g, 12.2 mmol) in THF (70.0 mL) at room temperature under an atmosphere of nitrogen. The reaction was cooled to 0° C. in an ice bath before dropwise addition of diisopropyl azodicarboxylate (4.71 mL, 24.3 mmol) in THF (10.00 mL). The reaction was stirred for 30 minutes at 0° C. and then warmed to room temperature for 60 minutes. The reaction mixture was diluted the water, ethyl acetate and saturated sodium bicarbonate solution. The aqueous layer was extracted with ethyl acetate. The combined organic fractions were washed with brine and then concentrated under reduced pressure. Purification by Biotage (Si, 50 g col, 0-100 ethyl acetate/hexanes) afforded the desired product as an off-white foam. (8.35 g, 10.3 mmol, yield: 85.1%) Compound 46. Compound 45, [(2R,3R,5R)-5-(6-benzamidopurin-9-yl)-2-[[bis(4-methoxyphenyl)-phenyl-methoxy]methyl]tetrahydrofuran-3-yl] 4-nitrobenzoate (8.35 g, 10.3 mmol) was dissolved in THF (69.1 mL) and then cooled to 0° C. in an ice bath. Sodium methoxide (0.500 M, 20.7 mL, 10.3 mmol) in Methanol was added and the reaction was stirred for 45 minutes at 0° C. The reaction mixture was dilute with water and ethyl acetate. The aqueous layer was extracted with ethyl acetate, followed by the combined organic fractions being washed with brine and concentrated to an oil. Purification by Biotage (Si, 220 g col, 0-100% ethyl acetate/hexanes) afforded the product as a white foam. (3.71 g, 5.64 mmol, yield: 54.5%).

Compound 47. Compound 46, N-[9-[(2R,4R,5R)-54[bis(4-methoxyphenyl)-phenyl-me thoxylmethyl]-4-hydroxy-tetrahydrofuran-2-yl]purin-6-yl]benzamide (3.71 g, 5.64 mmol) was dissolved in dry DMF (57.2 mL) under an atmosphere of nitrogen. To this was added 1H-TETRAZOLE (0.316 g, 4.51 mmol) and 1-Methylimidazole (0.112 mL, 1.41 mmol), followed by drop-wise addition of 2-Cyanoethyl N,N,N′,N′-tetraisopropylphosphorodiamidite (2.69 mL, 8.46 mmol). The reaction was stirred at room temperature for 90 minutes. Water (1.0 mL) was added to quench the reaction, followed by a 3:1 mixture of toluene/hexanes (80.0 mL). This organic fraction was washed four times with a 3:2 mixture of DMF/H2O (50.0 mL). The organic fraction was then washed with saturated sodium bicarbonate solution and brine, followed by drying over sodium sulfate. The crude reaction was then concentrated to an oil under reduced pressure. Purification by Biotage (Si, 220 g col, 0-100% ethyl acetate) afforded the desired product as a white solid. (1.65 g, 1.93 mmol, yield: 34.2%).

Compound 54, an amidite of a stereo-non-standard nucleoside, was prepared according to the scheme below:

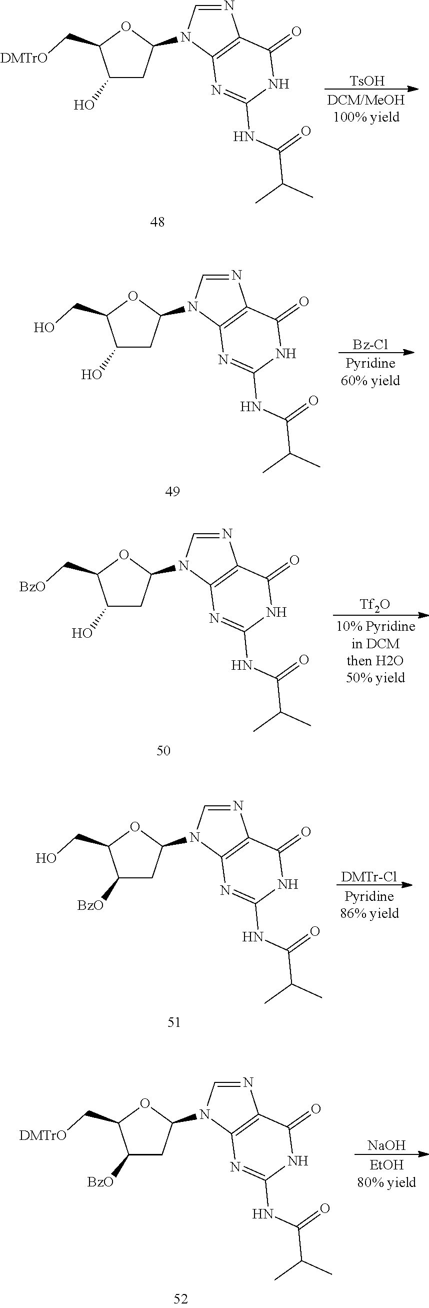

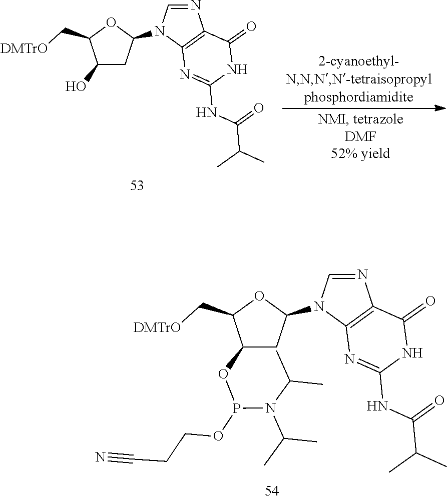

### Synthesis of Final Compound 54.

Compound 48 was obtained from a commercial supplier.

Compound 49. Compound 48, N-[9-[(2R,4S,5R)-5-[[bis(4-methoxyphenyl)-phenyl-methoxy]methyl]-4-hydroxy-tetrahydrofuran-2-yl]-6-oxo-1H-purin-2-yl]-2-methyl-propanamide (50.0 g, 78.2 mmol) was dissolved in DCM/Methanol (1560 mL) and cooled to 0° C. Sodium carbonate (9.94 g, 93.8 mmol) was added and the orange reaction mixture was stirred at 0° C. After 60 minutes sodium carbonate (9.94 g, 93.8 mmol) was added at 0° C. and stirred until the orange color disappeared. The solvents were removed under reduced pressure. Dichloromethane was added to the crude reaction and the white precipitate was isolated and dried under high vacuum. The crude desired product was isolated as a white solid. (28.7 g, 85.1 mmol, yield: 109%)

Compound 50. Crude compound 49, N-[9-[(2R,4S,5R)-4-hydroxy-5-(hydroxymethyl)tetrahydrofuran-2-yl]-6-oxo-1H-purin-2-yl]-2-methyl-propanamide (26.4 g, 78.3 mmol) was suspended in Pyridine (780 mL) under an atmosphere of nitrogen. Benzoyl chloride (9.08 mL, 78.3 mmol) was added drop-wise to the reaction and it stirred at room temperature for 1 hr. The solvents were removed under reduce pressure and the crude mixture was separated between dichloromethane and water. The organic phase was collected and washed with water (3 times) and brine. The crude reaction was then dried over sodium sulfate and concentrated under reduced pressure. Purification by Biotage (Si, 330 g col, 0-10% Methanol/Dichloromethane) afforded the desired product as a white solid. (20.7 g, 46.9 mmol, yield: 59.9%)

Compound 51. Compound 50, 2R,3S,5R)-3-hydroxy-5-[2-(2-methylpropanoylamino)-6-oxo-1H-purin-9-yl]tetrahydrofuran-2-yl]methyl benzoate (10.0 g, 0.0227 mol) was dissolved in 10% Pyridine in Dichloromethane (164 mL) and cooled to −35° C. in an acetone/dry ice bath under an atmosphere of nitrogen. Trifluoromethanesulfonic anhydride (5.72 mL, 0.0340 mol) was added drop-wise. After completion of addition the reaction mixture was warmed to 0° C. and stirred for 45 minutes before the addition of water (4.92 mL, 0.273 mol). The reaction was then warmed to room temperature overnight. The solvents were removed under reduced pressure. Equal volumes of water (150 mL) and ethyl acetate (150 mL) were added to the crude reaction and this was shaken in a separation funnel. The white precipitate formed collected and dried under high vacuum affording the desired product as a white solid. (5.24 g, 0.0119 mol, yield: 52.4%)

Compound 52. DMT-Cl (3.68 g, 10.9 mmol) was added to a solution of Compound 51, [(2R,3R,5R)-2-(hydroxymethyl)-5-[2-(2-methylpropanoylamino)-6-oxo-1H-purin-9-yl]tetrahydrofuran-3-yl] benzoate (4.00 g, 9.06 mmol) in Pyridine (30.2 mL) and the reaction was stirred at room temperature for 2 hours. The reaction was concentrated to an oil and purification by Biotage (Si, 10 g col, 0-100% ethyl acetate/hexanes) afforded the desired product as a white solid. (5.79 g, 7.78 mmol, yield: 85.9%)

Compound 53. Compound 52, [(2R,3R,5R)-2-[[bis(4-methoxyphenyl)-phenyl-methoxy]methyl]-5-[2-(2-methylpropanoylamino)-6-oxo-1H-purin-9-yl]tetrahydrofuran-3-yl]benzoate (5.45 g, 0.00733 mol) was dissolved in a 1:1:1 mixture of THF (54.5 mL):1,4-Dioxane (54.5 mL):Methanol (54.5 mL). The reaction was cooled to 0° C. and to this was added 1 N NaOH (54.5 mL). The reaction was stirred at 0° C. for 2 hours. The reaction was then diluted with ethyl acetate and water. The aqueous fraction was extracted with ethyl acetate. The combined organic fractions were washed with brine and dried over sodium sulfate. Purification by Biotage (Si, 10 g col, 0-5% methanol/methanol) afforded the desired product as a white solid. (3.73 g, 0.00583 mol, yield: 79.6%)

Compound 54. Compound 53, N-[9-[(2R,4R,5R)-54[bis(4-methoxyphenyl)-phenyl-methoxy]methyl]-4-hydroxy-tetrahydrofuran-2-yl]-6-oxo-1H-purin-2-yl]-2-methyl-propanamide (3.00 g, 4.69 mmol) was dissolved in dry DMF (46.8 mL) under an atmosphere of nitrogen. To this was added 1H-Tetrazole (0.263 g, 3.75 mmol) and 1-Methylimidazole (0.0930 mL, 1.17 mmol), followed by drop-wise addition of 2-Cyanoethyl N,N,N′,N′-tetraisopropylphosphorodiamidite (2.23 mL, 7.03 mmol). This was stirred at room temperature overnight. Water (1.0 mL) was added to quench the reaction, followed by a 3:1 mixture of toluene/hexanes (80.0 mL). This organic fraction was washed four times with a 3:2 mixture of DMF/H2O (50.0 mL). The organic fraction was then washed with saturated sodium bicarbonate solution and brine, followed by drying over sodium sulfate. The crude reaction was then concentrated to an oil under reduced pressure. Purification by Biotage (Si, 50 g col, 0-100% ethyl acetate) afforded the desired product as a white solid. (2.03 g, 2.42 mmol, yield: 51.5%.)

Compound 62, an amidite of a 2′substituted stereo-non-standard nucleoside, was prepared according to the scheme below:

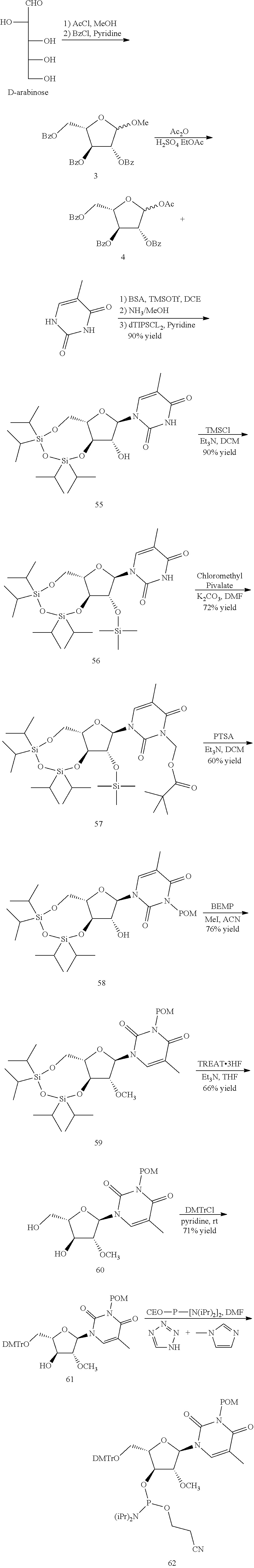

### Example 17: Design and Synthesis of 2′-Substituted Stereo-Standard Nucleosides, Stereo-Non-Standard Nucleosides, and 2′-Substituted Stereo-Non-Standard Nucleosides

2′-substituted stereo-non-standard nucleosides and stereo-non-standard nucleosides described herein may be prepared as amidites as described below. The 2′-substituted stereo-non-standard nucleoside amidites and stereo-non-standard nucleoside amidites may then be incorporated into a modified oligonucleotide during modified oligonucleotide synthesis.

A scheme for the synthesis of an amidite of the stereo-non-standard nucleoside 63 is shown below:

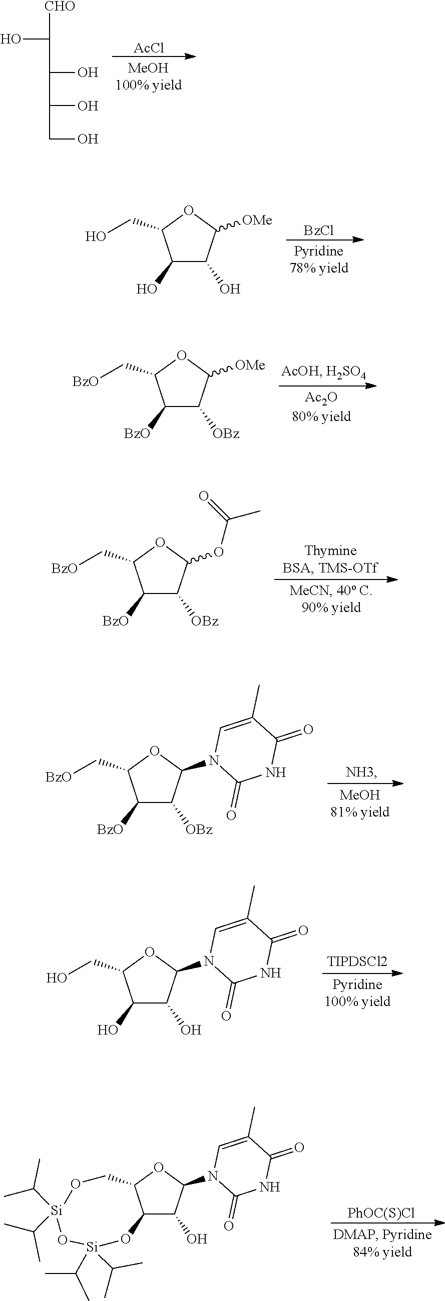

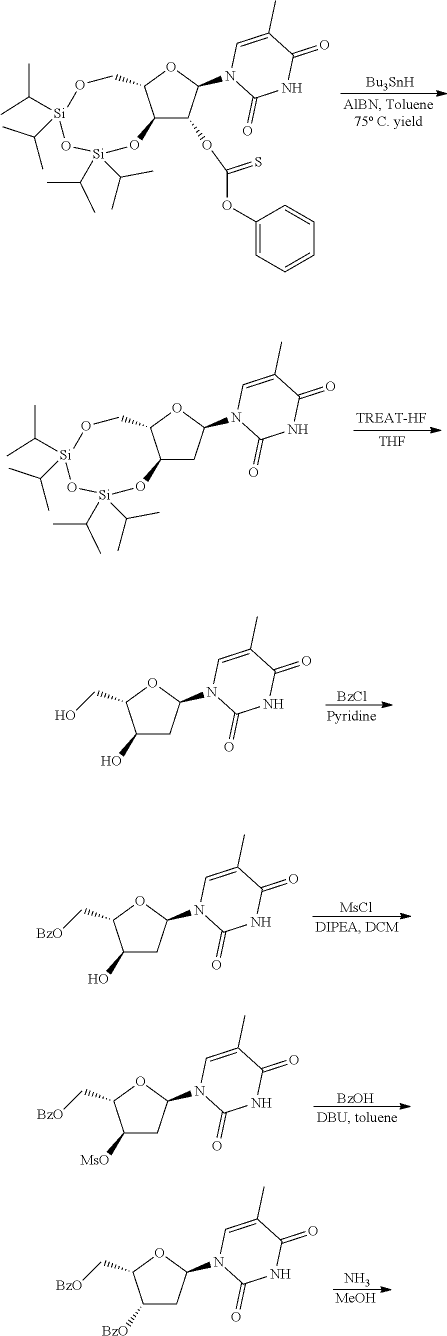

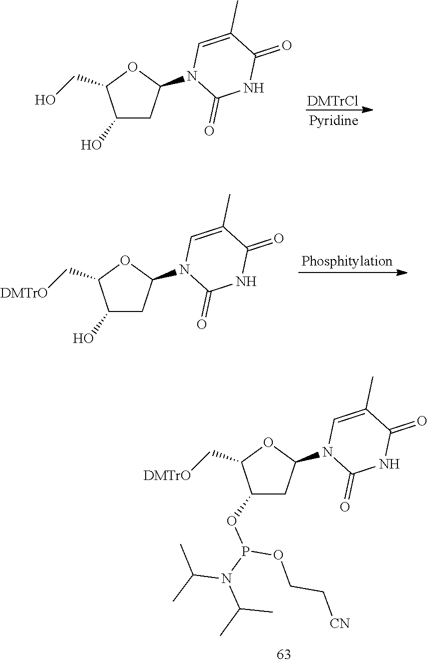

A scheme for the synthesis of an amidite of the stereo-non-standard nucleoside 64 is shown below:

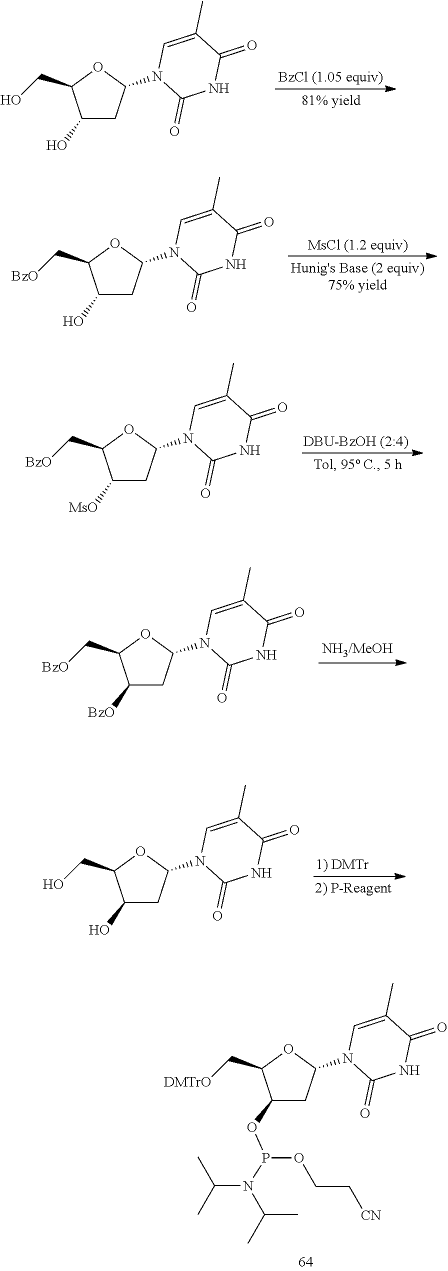

A scheme for the synthesis of an amidite of the stereo-non-standard nucleoside 65 is shown below:

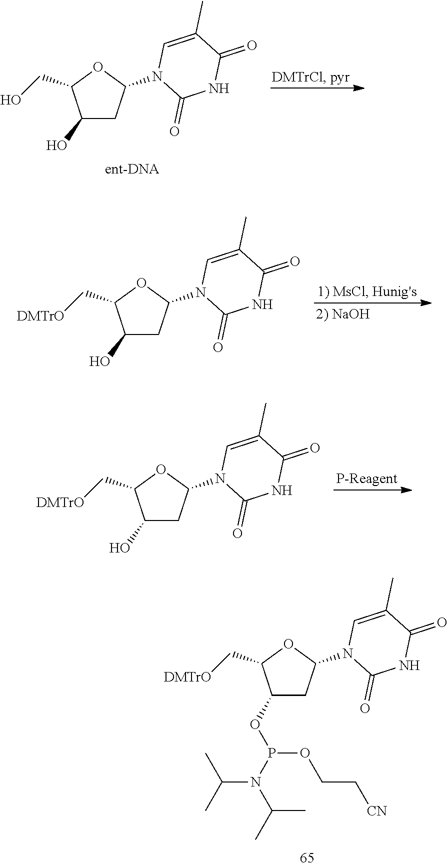

A scheme for the synthesis of an amidite of the 2′ substituted stereo-standard nucleoside 66 is shown below:

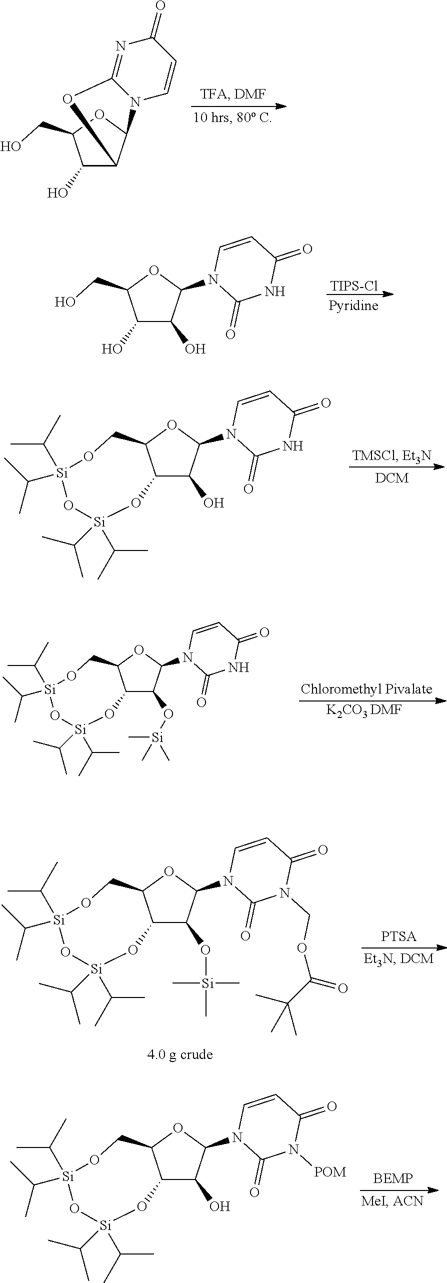

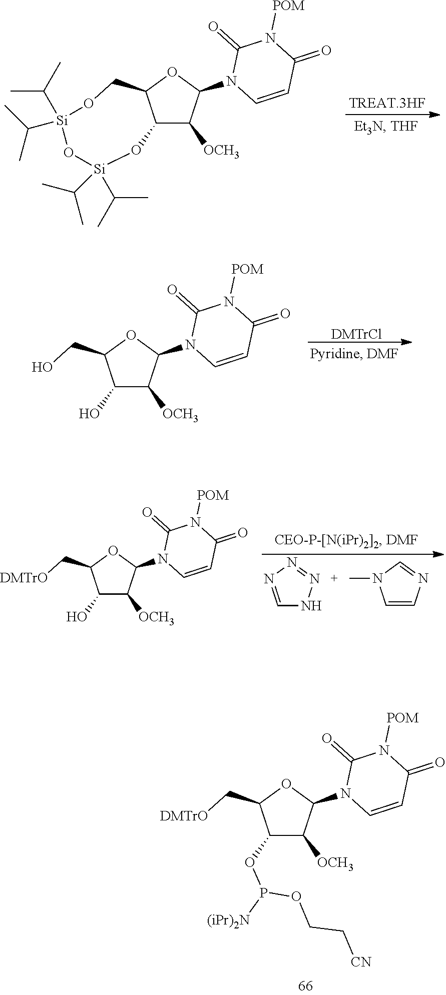

Schemes for the synthesis of amidites of the 2′substituted stereo-non-standard nucleosides 67, 68, and 69 are shown below:

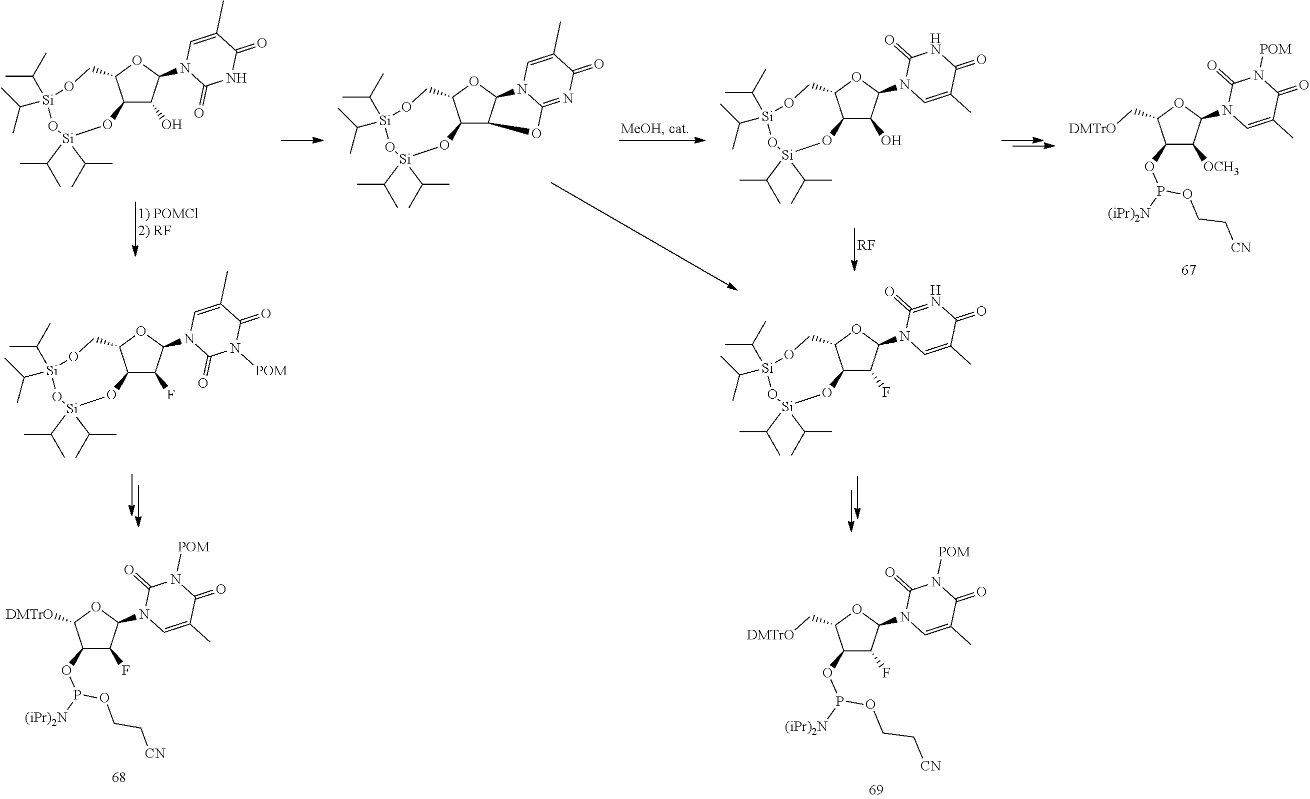

A scheme for the synthesis of an amidite of the 2′ substituted stereo-non-standard nucleoside 70 is shown below:

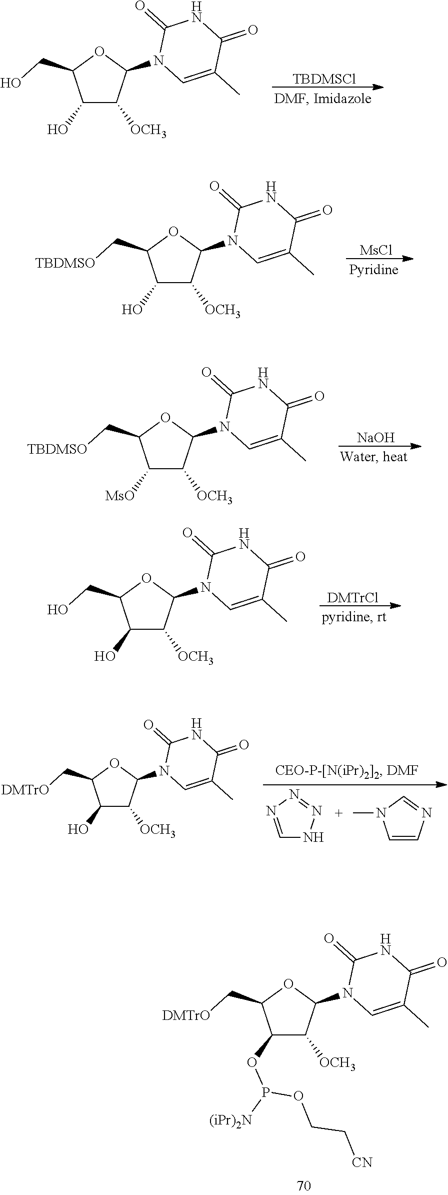

A scheme for the synthesis of an amidite of the 2′ substituted stereo-non-standard nucleoside 71 is shown below:

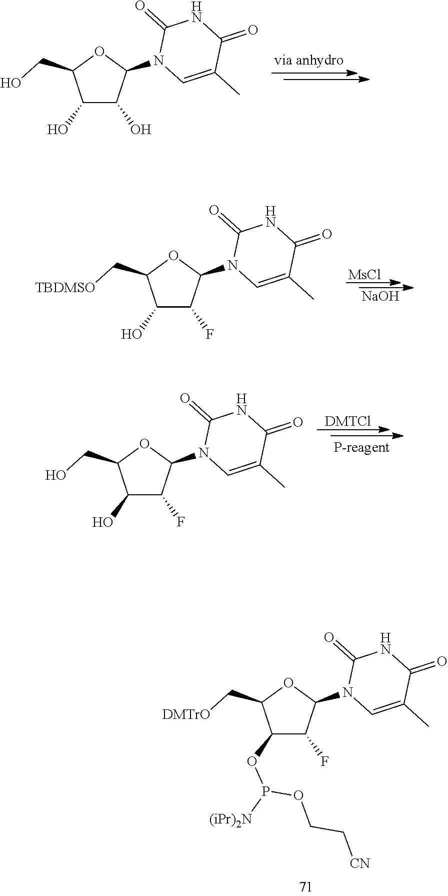

1.-202. (canceled)

203. An oligomeric compound comprising a modified oligonucleotide consisting of 15-30 linked nucleosides, wherein at least one nucleoside of the modified oligonucleotide is a stereo-non-standard nucleoside, wherein at least one stereo-non-standard nucleoside has the structure of Formula II or of Formula V: 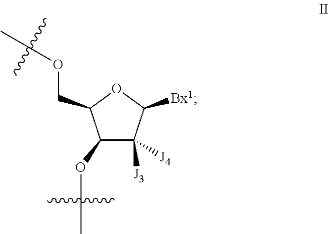
wherein one of J₃ and J₄ is H and the other of J₃ and J₄ is selected from H, OH, F, OCH₃, OCH₂CH₂OCH₃, O—C₁-C₆ alkoxy, and SCH₃; and wherein
Bx¹ is a is a heterocyclic base moiety; 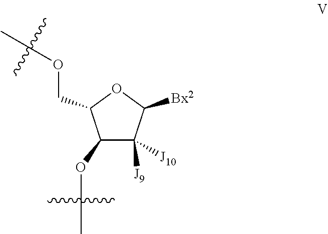
wherein one of J₉ and J₁₀ is H and the other of J₉ and J₁₀ is selected from H, OH, F, OCH₃, OCH₂CH₂OCH₃, O—C₁-C₆ alkoxy, and SCH₃; and wherein
Bx² is a is a heterocyclic base moiety.

204. The oligomeric compound of claim 203, wherein the wherein the modified oligonucleotide comprises a deoxy region consisting of 5-12 contiguous nucleosides, wherein:
each nucleoside of the deoxy region is selected from a stereo-standard DNA nucleoside, a stereo-non-standard nucleoside, and a substituted stereo-standard nucleoside;
the 5′-most nucleoside and 3′-most nucleoside of the deoxy region are not substituted stereo-standard nucleosides;
at least one nucleoside of the deoxy region is a stereo-non-standard nucleoside; and
not more than one nucleoside of the deoxy region is a substituted stereo-standard nucleoside.

205. The oligomeric compound of claim 204, wherein the 3′-most nucleoside of the deoxy region is a stereo-standard DNA nucleoside.

206. The oligomeric compound of claim 204, wherein exactly 1 or exactly 2 nucleosides of the deoxy region are stereo-non-standard nucleosides having Formula II or Formula V.

207. The oligomeric compound of claim 206, wherein the remainder of the nucleosides of the deoxy region are stereo-standard DNA nucleosides.

208. The oligomeric compound of claim 204, wherein the 5′-most nucleoside of the deoxy region is a stereo-non-standard nucleoside.

209. The oligomeric compound of claim 204, wherein the 2nd deoxy region nucleoside from the 5′-end of the deoxy region is a stereo-non-standard nucleoside.

210. The oligomeric compound of claim 204, wherein the deoxy region consists of 8-10 linked nucleosides and is flanked on the 5′ side by a 5′-region consisting of 1-6 linked 5′-region nucleosides and on the 3′ side by a 3′-region consisting of 1-6 linked 3′-region nucleosides; wherein
each 5′-region nucleoside is a 2′-substituted stereo-standard nucleoside or a bicyclic nucleoside, and each 3′-region nucleoside is a 2′-substituted stereo-standard nucleoside or a bicyclic nucleoside.

211. The oligomeric compound of claim 210, wherein each 2′-substituted stereo-standard 5′-region nucleoside has a 2′-substituent selected from: 2′-F, 2′-OCH3, 2′-MOE, 2′-NMA.

212. The oligomeric compound of claim 210, wherein each bicyclic 5′-region nucleoside is selected from among a cEt nucleoside, a β-D-LNA nucleoside, an α-L-LNA nucleoside, and an ENA nucleoside.

213. The oligomeric compound of claim 212, wherein each 5′-region nucleoside is a bicyclic nucleoside.

214. The oligomeric compound of claim 210, wherein each 2′-substituted stereo-standard 3′-region nucleoside has a 2′-substituent selected from: 2′-F, 2′-OCH3, 2′-MOE, 2′-NMA.

215. The oligomeric compound of claim 210, wherein each bicyclic 3′-region nucleoside is selected from among a cEt nucleoside, a β-D-LNA nucleoside, an α-L-LNA nucleoside, and an ENA nucleoside.

216. The oligomeric compound of claim 215, wherein each 3′-region nucleoside is a bicyclic nucleoside.

217. The oligomeric compound of claim 203, wherein at least one internucleoside linkage is a phosphorothioate internucleoside linkage.

218. The oligomeric compound of claim 203, wherein at least one internucleoside linkage is a phosphodiester internucleoside linkage.

219. The oligomeric compound of claim 203, wherein each internucleoside linkage is either a phosphorothioate internucleoside linkage or a phosphodiester internucleoside linkage.

220. The oligomeric compound of claim 203, comprising a conjugate group.

221. The oligomeric compound of claim 203, wherein the modified oligonucleotide is single-stranded.

222. An oligomeric duplex comprising the oligomeric compound of claim 203, and a second oligomeric compound comprising a second modified oligonucleotide.

223. The oligomeric compound of claim 203, wherein the nucleobase sequence of the modified oligonucleotide is at least 80%, at least 85%, at least 90%, at least 95%, or 100% complementary to an equal length portion of a target nucleic acid selected from mRNA and pre-mRNA.

224. The oligomeric compound of claim 203, wherein the at least one stereo-nonstandard nucleoside has the structure of Formula II, wherein J₃ and J₄ are each H.

225. The oligomeric compound of claim 203, wherein the at least one stereo-nonstandard nucleoside has the structure of Formula V, wherein J₉ and J₁₀ are each H.

226. The oligomeric compound of claim 203, wherein the at least one stereo-nonstandard nucleoside has the structure of Formula V, wherein J₉ is 2′-OMe and J₁₀ is H.

227. The oligomeric compound of claim 203, wherein each Bx is independently selected from uracil, thymine, cytosine, 5-methyl cytosine, adenine or guanine.
